(19) United States
(12) Patent Application Publication
(10) Pub. No.: US 2023/0310413 A1
(43) Pub. Date: Oct. 5, 2023

(54) PHARMACEUTICAL FORMULATIONS COMPRISING A MALT1 INHIBITOR AND A MIXTURE OF POLYETHYLENE GLYCOL WITH A FATTY ACID

(71) Applicant: JANSSEN PHARMACEUTICA NV, Beerse (BE)

(72) Inventors: Sanket Manoj SHAH, Antwerpen (BE); Donghua ZHU, Shanghai (CN); René HOLM, Woluwe Saint Pierre (BE); Kristof Leonard KIMPE, Beerse (BE)

(21) Appl. No.: 18/041,831

(22) PCT Filed: Aug. 20, 2021
(86) PCT No.: PCT/CN2021/113678

§ 371 Date: Feb. 16, 2023

(30) Foreign Application Priority Data
Aug. 21, 2020 (WO) PCT/CN2020/110403

(51) Int. Cl. A61K 31/4725; A61K 9/48

(52) U.S. Cl. CPC A61K 31/4725; A61K 9/4858; A61K 9/4825

(57) ABSTRACT

Described herein are pharmaceutical formulations comprising a MALT1 inhibitor and a mixture comprising fatty acid and polyethylene glycol monoesters and diesters, and optionally, fatty acid and glycerol monoesters, diesters and triesters. Solid dosage forms comprising said pharmaceutical formulations, processes for preparing these and their use in methods of treatment are also described.

### FIELD OF THE INVENTION

The present invention relates to pharmaceutical formulations comprising a MALT1 inhibitor and a mixture comprising fatty acid and polyethylene glycol monoesters and diesters, and optionally, fatty acid and glycerol monoesters, diesters and triesters. The invention also relates to solid dosage forms comprising said pharmaceutical formulations, to processes to prepare such pharmaceutical formulations, and to the use of such pharmaceutical formulations for the treatment of a disease, syndrome, condition, or disorder.

### BACKGROUND OF THE INVENTION

Many active pharmaceutical ingredients (API) have properties such as hydrophobicity and instability leading to challenges in providing suitable pharmaceutical formulations.

MALT1 (mucosa-associated lymphoid tissue lymphoma translocation 1) is a key mediator of the classical NFKB signaling pathway. WO 2018/119036 discloses a class of active pharmaceutical agents which are MALT1 inhibitors that may provide a therapeutic benefit to patients suffering from cancer and/or immunological diseases.

There exists a need for improved pharmaceutical formulations of active pharmaceutical ingredients, such as the MALT1 inhibitors described in WO 2018/119036. In particular there exists a need for pharmaceutical formulations with an acceptable bio-availability, in particular in a solid dosage form.

### SUMMARY OF THE INVENTION

Described herein are pharmaceutical formulations, comprising a first component and a second component; wherein the first component is an active pharmaceutical ingredient which is a MALT1 inhibitor and the second component is a mixture comprising fatty acid and polyethylene glycol monoesters and diesters, and optionally, fatty acid and glycerol monoesters, diesters and triesters; wherein the fatty acid component of the fatty acid and polyethylene glycol monoesters and diesters, and of the fatty acid and glycerol monoesters, diesters and triesters, when present, comprises one or more saturated fatty acids having at least eight carbons.

The invention provides a pharmaceutical formulation, comprising a first component and a second component;
wherein the first component is an active pharmaceutical ingredient which is a compound as described herein, for example a compound of Formula (I) as described herein, for example a compound of Formula (I):

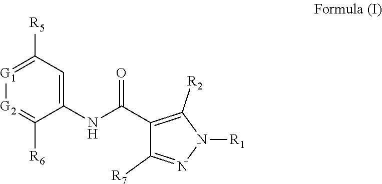

wherein
   R₁ is selected from the group consisting of
i) naphthalen-1-yl, optionally substituted with a fluoro or amino substituent;
   and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
   R₂ is selected from the group consisting of C₁₋₄alkyl, 1-methoxy-ethyl, difluoromethyl, fluoro, chloro, bromo, cyano, and trifluoromethyl;
   G₁ is N or C(R₄);
   G₂ is N or C(R₃); such that only one of G₁ and G₂ are N in any instance;
   R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
   R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy;
   and
xviii) morpholin-4-ylcarbonyl;
   R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
   or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
   R₆ is hydrogen, C₁₋₄alkyl, fluoro, 2-methoxy-ethoxy, chloro, cyano, or trifluoromethyl; and
   R₇ is hydrogen or fluoro;
or an enantiomer, diastereomer, solvate, or pharmaceutically acceptable salt form thereof, and
wherein the second component is a mixture comprising fatty acid and polyethylene glycol monoesters and diesters, and optionally, fatty acid and glycerol monoesters, diesters and triesters;
wherein the fatty acid component of the fatty acid and polyethylene glycol monoesters and diesters, and of the fatty acid and glycerol monoesters, diesters and triesters, when present, comprises one or more saturated fatty acids having at least eight carbons.

The invention also provides a solid dosage form comprising a pharmaceutical formulation described herein.

The invention provides methods for treating or ameliorating a disease, syndrome, condition, or disorder in a subject, including a mammal and/or human in which the disease, syndrome, condition, or disorder is affected by the inhibition of MALT1, including but not limited to, cancer and/or immunological diseases, using pharmaceutical formulations and solid dosage forms described herein.

The present invention is also directed to the use of such pharmaceutical formulations in the preparation of a medicament wherein the medicament is prepared for treating a disease, syndrome, disorder or condition that is affected by the inhibition of MALT1, such as cancer and/or immunological diseases.

Exemplifying the invention are methods of treating a disease, syndrome, condition, or disorder mediated by MALT1, selected from the group consisting of lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor), comprising, consisting of, and/or consisting essentially of, administering to a subject in need thereof a therapeutically effective amount of the pharmaceutical formulation or solid dosage form.

In another embodiment, the present invention is directed to pharmaceutical formulations and solid dosage forms described herein for use in the treatment of a disease, syndrome, condition, or disorder affected by the inhibition of MALT1, such as cancer and/or immunological disease. The disease, syndrome, condition, or disorder may be selected from the group consisting of lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

The invention also provides a process for preparing a solid or semi-solid pharmaceutical formulation described herein, the process comprising the steps of:
a) forming a melt comprising a first component and a second component, wherein
the forming a melt step comprises heating the second component; and
b) cooling the melt;
   to provide a solid or semi-solid pharmaceutical formulation described herein;
wherein the first component is an active pharmaceutical ingredient which is a compound as described herein, for example a compound of Formula (I), as described herein, or an enantiomer, diastereomer, solvate, or pharmaceutically acceptable salt form thereof, and
wherein the second component is a mixture comprising fatty acid and polyethylene glycol monoesters and diesters, and optionally, fatty acid and glycerol monoesters, diesters and triesters;
wherein the fatty acid component of the fatty acid and polyethylene glycol monoesters and diesters, and of the fatty acid and glycerol monoesters, diesters and triesters, when present, comprises one or more saturated fatty acids having at least eight carbons.

The invention also provides a process for preparing a solid dosage form described herein, the process comprising the steps of:
a) forming a melt comprising a first component and a second component, wherein
the forming a melt step comprises heating the second component;
b) filling a capsule with the melt; and
c) cooling the filled capsule;
   to provide a solid dosage form described herein;
wherein the first component is an active pharmaceutical ingredient which is a compound as described herein, for example a compound of Formula (I), as described herein, or an enantiomer, diastereomer, solvate, or pharmaceutically acceptable salt form thereof, and
wherein the second component is a mixture comprising fatty acid and polyethylene glycol monoesters and diesters, and optionally, fatty acid and glycerol monoesters, diesters and triesters;
wherein the fatty acid component of the fatty acid and polyethylene glycol monoesters and diesters, and of the fatty acid and glycerol monoesters, diesters and triesters, when present, comprises one or more saturated fatty acids having at least eight carbons.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific disclosure of the drawings. In the drawings:

FIG. 1 is an X-ray powder diffraction (XRPD) pattern of the crystalline form of Compound A hydrate as obtained in Example 1.

FIG. 2 is an X-ray powder diffraction (XRPD) pattern of the crystalline form of Compound A monohydrate as obtained in Example 3.

FIG. 3 shows the results of a physiology-based dissolution test (PBDT) using various capsule formulations of Compound A monohydrate with polyoxyl-32 stearate type I (Gelucire® 48/16).

### DETAILED DESCRIPTION OF THE INVENTION

The disclosure may be more fully appreciated by reference to the following description, including the following glossary of terms and the concluding examples. It is to be appreciated that certain features of the disclosed pharmaceutical formulations, solid dosage forms, uses and methods which are, for clarity, described herein in the context of separate aspects, may also be provided in combination in a single aspect. Conversely, various features of the disclosed pharmaceutical formulations, solid dosage forms, uses and methods that are, for brevity, described in the context of a single aspect, may also be provided separately or in any sub-combination.

Some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and claims of this specification, the words “comprise” and “contain” and variations of the words, for example “comprising” and “comprises”, mean “including but not limited to”, and are not intended to (and do not) exclude other components.

With reference to substituents, the term “independently” refers to the situation where when more than one substituent is possible, the substituents may be the same or different from each other.

The term “aliphatic” refers to a straight-chain, branched or cyclic hydrocarbon, which is completely saturated or which contains one or more units of unsaturation, but which is not aromatic. Aliphatic groups include linear, branched, or cyclic alkyl, alkenyl, alkynyl groups and hybrids thereof, such as (cycloalkyl)alkyl, (cycloalkenyl)alkyl or (cycloalkyl)alkenyl. An aliphatic group may have 1 to 40, 1 to 30, or 1 to 20 carbons.

The term “alkyl” whether used alone or as part of a substituent group, refers to straight and branched carbon chains having, for example, 1 to 8 carbon atoms. Therefore, designated numbers of carbon atoms (e.g., C₁₋₈) refer independently to the number of carbon atoms in an alkyl moiety or to the alkyl portion of a larger alkyl-containing substituent. In substituent groups with multiple alkyl groups such as, (C₁₋₆alkyl)₂ amino-, the C₁₋₆alkyl groups of the dialkylamino may be the same or different.

The term “alkoxy” refers to an —O-alkyl group, wherein the term “alkyl” is as defined above.

The terms “alkenyl” and “alkynyl” refer to straight and branched carbon chains having, for example, 2 to 8 carbon atoms, wherein an alkenyl chain contains at least one double bond and an alkynyl chain contains at least one triple bond.

The term “cycloalkyl” refers to saturated or partially saturated, monocyclic or polycyclic hydrocarbon rings of, for example, 3 to 14 carbon atoms. Examples of such rings include cyclopropyl, cyclobutyl, cyclopentyl, cyclohexyl, cycloheptyl, and adamantyl.

The term “heterocyclyl” refers to a nonaromatic monocyclic or bicyclic ring system having 3 to 10 ring members that include at least 1 carbon atom and from 1 to 4 heteroatoms independently selected from N, O, and S. Included within the term heterocyclyl is a nonaromatic cyclic ring of 5 to 7 members in which 1 to 2 members are N, or a nonaromatic cyclic ring of 5 to 7 members in which 0, 1 or 2 members are N and up to 2 members are O or S and at least one member must be either N, O, or S; wherein, optionally, the ring contains 0 to 1 unsaturated bonds, and, optionally, when the ring is of 6 or 7 members, it contains up to 2 unsaturated bonds. The carbon atom ring members that form a heterocycle ring may be fully saturated or partially saturated. The term “heterocyclyl” also includes two 5 membered monocyclic heterocycloalkyl groups bridged to form a bicyclic ring. Such groups are not considered to be fully aromatic and are not referred to as heteroaryl groups. When a heterocycle is bicyclic, both rings of the heterocycle are non-aromatic and at least one of the rings contains a heteroatom ring member. Examples of heterocycle groups include, and are not limited to, pyrrolinyl (including 2H-pyrrole, 2-pyrrolinyl or 3-pyrrolinyl), pyrrolidinyl, imidazolinyl, imidazolidinyl, pyrazolinyl, pyrazolidinyl, piperidinyl, morpholinyl, thiomorpholinyl, and piperazinyl. Unless otherwise noted, the heterocycle is attached to its pendant group at any heteroatom or carbon atom that results in a stable structure.

The term “aryl” refers to an unsaturated, aromatic monocyclic or bicyclic ring of 6 to 10 carbon members. Examples of aryl rings include phenyl and naphthalenyl.

The term “heteroaryl” refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 3 nitrogen atoms. For the case wherein the 6 membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, oxazolyl, thiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

The term “halogen” or “halo” refers to fluorine, chlorine, bromine and iodine atoms.

The term “carboxy” refers to the group —C(═O)OH.

The term “formyl” refers to the group —C(═O)H.

The term “oxo” or “oxido” refers to the group (═O).

Whenever the term “alkyl” or “aryl” or either of their prefix roots appear in a name of a substituent (e.g., arylalkyl, alkylamino) the name is to be interpreted as including those limitations given above for “alkyl” and “aryl.” Designated numbers of carbon atoms (e.g., C₁-C₆) refer independently to the number of carbon atoms in an alkyl moiety, an aryl moiety, or in the alkyl portion of a larger substituent in which alkyl appears as its prefix root. For alkyl and alkoxy substituents, the designated number of carbon atoms includes all of the independent members included within a given range specified. For example C₁₋₆ alkyl would include methyl, ethyl, propyl, butyl, pentyl and hexyl individually as well as sub-combinations thereof (e.g., C₁₋₂, C₁₋₃, C₁₋₄, C₁₋₅, C₂₋₆, C₃₋₆, C₄₋₆, C₅₋₆, C₂₋₅, etc.).

In general, under standard nomenclature rules used throughout this disclosure, the terminal portion of the designated side chain is described first followed by the adjacent functionality toward the point of attachment. Thus, for example, a “C₁-C₆ alkylcarbonyl” substituent refers to a group of the formula:

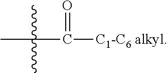

The label “R” at a stereocenter designates that the stereocenter is purely of the R-configuration as defined in the art; likewise, the label “S” means that the stereocenter is purely of the S-configuration. As used herein, the labels “*R” or “*S” at a stereocenter are used to designate that the stereocenter is of pure but unknown absolute configuration. As used herein, the label “RS” refers to a stereocenter that exists as a mixture of the R- and S-configurations.

A compound containing one stereocenter drawn without a stereo bond designation is a mixture of two enantiomers. A compound containing two stereocenters both drawn without stereo bond designations is a mixture of four diastereomers. A compound with two stereocenters both labeled “RS” and drawn with stereo bond designations is a mixture of two enantiomers with relative stereochemistry as drawn. A compound with two stereocenters both labeled “*RS” and drawn with stereo bond designations is a mixture of two enantiomers with a single, but unknown, relative stereochemistry.

Unlabeled stereocenters drawn without stereo bond designations are mixtures of the R- and S-configurations. For unlabeled stereocenters drawn with stereo bond designations, the relative and absolute stereochemistry is as depicted.

Unless otherwise noted, it is intended that the definition of any substituent or variable at a particular location in a molecule be independent of its definitions elsewhere in that molecule. It is understood that substituents and substitution patterns on the compounds of the present invention can be selected by one of ordinary skill in the art to provide compounds that are chemically stable and that can be readily synthesized by techniques known in the art as well as those methods set forth herein.

A person of ordinary skill in the art would recognize that the compounds described herein may exist as tautomers and that other tautomeric arrangements of the structures depicted herein are possible. It is understood that all tautomeric forms are encompassed by a structure where one possible tautomeric arrangement of the groups of the compound is described, even if not specifically indicated.

For example, it is understood that

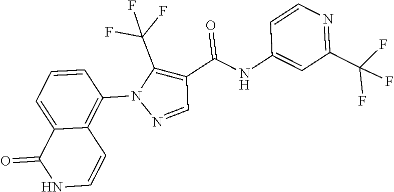 [SMILES: O=C(Nc1ccnc(C(F)(F)F)c1)c1cnn(-c2cccc3c(=O)[nH]ccc23)c1C(F)(F)F]

also encompasses by the following structure

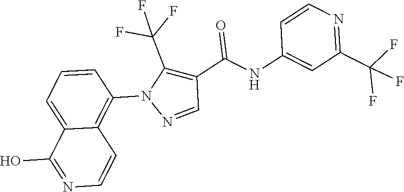 [SMILES: O=C(Nc1ccnc(C(F)(F)F)c1)c1cnn(-c2cccc3c(O)nccc23)c1C(F)(F)F]

Any convenient tautomeric arrangement may be utilized in describing the compounds.

For use in medicine, salts of compounds of Formula (I) refer to non-toxic “pharmaceutically acceptable salts.” “Pharmaceutically acceptable” may mean approved or approvable by a regulatory agency of the Federal or a state government or the corresponding agency in countries other than the United States, or that is listed in the U. S. Pharmacopoeia or other generally recognized pharmacopoeia for use in animals, and more particularly, in humans.

Other salts may, however, be useful in the preparation of compounds of Formula (I) or of their pharmaceutically acceptable salt forms thereof. Suitable pharmaceutically acceptable salts of compounds of Formula (I) include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid or phosphoric acid. Furthermore, where the compounds of Formula (I) carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

Representative acids and bases that may be used in the preparation of pharmaceutically acceptable salts include acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine, and zinc hydroxide.

Embodiments of the present invention include prodrugs of compounds of Formula (I). In general, such prodrugs will be functional derivatives of the compounds that are readily convertible in vivo into the required compound. Thus, in the methods of treating or preventing embodiments of the present invention, the term “administering” encompasses the treatment or prevention of the various diseases, conditions, syndromes and disorders described with the compound specifically disclosed or with a compound that may not be specifically disclosed, but which converts to the specified compound in vivo after administration to a patient. Conventional procedures for the selection and preparation of suitable prodrug derivatives are described, for example, in “Design of Prodrugs”, ed. H. Bundgaard, Elsevier, 1985.

Where the compounds of Formula (I) have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the compounds may exist as polymorphs and as such are intended to be included in the present invention. In addition, some of the compounds may form solvates with water (i.e., hydrates) or common organic solvents, and such solvates are also intended to be encompassed within the scope of this invention. Solvates may be pharmaceutically acceptable solvates. The skilled artisan will understand that the term compound as used herein, is meant to include solvated compounds of Formula (I).

Where the processes for the preparation of the compounds of Formula (I) give rise to mixture of stereoisomers, these isomers may be separated by conventional techniques such as, preparative chromatography. The compounds may be prepared in racemic form, or individual enantiomers may be prepared either by enantiospecific synthesis or by resolution. The compounds may, for example, be resolved into their component enantiomers by standard techniques such as, the formation of diastereomeric pairs by salt formation with an optically active acid such as, (−)-di-p-toluoyl-d-tartaric acid and/or (+)-di-p-toluoyl-1-tartaric acid followed by fractional crystallisation and regeneration of the free base. The compounds may also be resolved by formation of diastereomeric esters or amides, followed by chomatographic separation and removal of the chiral auxiliary. Alternatively, the compounds may be resolved using a chiral HPLC column.

In one embodiment of the pharmaceutical formulation of the present invention, the API (e.g. a compound of Formula (I)) is a compound comprising, consisting of, and/or consisting essentially of the (+)-enantiomer wherein said compound is substantially free from the (−)-isomer. In the present context, substantially free means less than about 25%, preferably less than about 10%, more preferably less than about 5%, even more preferably less than about 2% and even more preferably less than about 1% of the (−)-isomer calculated as

$$\% ( + ) - enantiomer = \frac{( mass ( + ) - enantiomer )}{( mass ( + ) - enantiomer ) + ( mass ( - ) - enantiomer )} \times 100.$$

In another embodiment of the pharmaceutical formulation of the present invention, the API (e.g. a compound of Formula (I)) is a compound comprising, consisting of, and consisting essentially of the (−)-enantiomer wherein said compound is substantially free from the (+)-isomer. In the present context, substantially free from means less than about 25%, preferably less than about 10%, more preferably less than about 5%, even more preferably less than about 2% and even more preferably less than about 1% of the (+)-isomer calculated as

$$\% ( - ) - enantiomer = \frac{( mass ( - ) - enantiomer )}{( mass ( + ) - enantiomer ) + ( mass ( - ) - enantiomer )} \times 100.$$

It is intended that within the scope of the present invention, any one or more element(s), in particular when mentioned in relation to a compound of Formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and 180. The isotopes may be radioactive or non-radioactive. Radiolabelled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ²H, ³H, ¹¹C and ¹⁸F.

During any of the processes for preparation of the compounds of the various embodiments of the present invention, it may be necessary and/or desirable to protect sensitive or reactive groups on any of the molecules concerned. This may be achieved by means of conventional protecting groups such as those described in Protective Groups in Organic Chemistry, Second Edition, J. F. W. McOmie, Plenum Press, 1973; T. W. Greene & P. G. M. Wuts, Protective Groups in Organic Synthesis, John Wiley & Sons, 1991; and T. W. Greene & P. G. M. Wuts, Protective Groups in Organic Synthesis, Third Edition, John Wiley & Sons, 1999. The protecting groups may be removed at a convenient subsequent stage using methods known from the art.

The term “room temperature” (RT) refers to a temperature of from about 15° C. to about 30° C., in particular from about 20° C. to about 30° C. Preferably, room temperature is a temperature of about 25° C.

The term “fatty acid” refers to a carboxylic acid having an aliphatic chain and a terminal carboxyl group. The aliphatic chain may alternatively be referred to as the fatty acid tail. A fatty acid may be a saturated fatty acid (i.e. wherein the aliphatic chain is an alkyl) or an unsaturated fatty acid (i.e. wherein the aliphatic chain contains at least one —C═C— or —C≡C— bond). Where a —C═C— bond is present, this may have cis (Z) or trans (E) stereochemistry.

A fatty acid may be defined by the number of carbon atoms present, including the carbons of the aliphatic chain and the carboxyl group. For example, lauric acid (CH₃(CH₂)₁₀COOH) is a fatty acid having 12 carbons and can be referred to as a C12 fatty acid. A fatty acid may also be defined by the number of carbon atoms present and the number of unsaturated bonds present. For example, lauric acid can be referred to as C12:0 and α-linolenic acid (CH₃CH₂CH═CHCH₂CH═CHCH₂CH═CH(CH₂)₇COOH) can be referred to as C18:3.

A fatty acid may have at least 4 carbons. A fatty acid may have at most 40 carbons. A fatty acid may have from 4 to 40 carbons, from 8 to 30 carbons, or from 8 to 20 carbons. The aliphatic chain may be an unbranched chain. The aliphatic chain may be an alkyl or alkenyl chain. A fatty acid may have an even number of carbon atoms.

Suitable examples of a saturated fatty acid include, but are not limited to, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, begenic acid, lignoceric acid, and cerotic acid.

The term “fatty acid and polyethylene glycol monoester” refers to an ester derived from a fatty acid molecule and a polyethylene glycol molecule, represented by

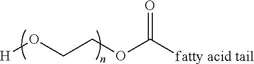

wherein n refers to the number of ethylene oxide units (—O—CH₂—CH₂—) per polyethylene glycol molecule.

The term “fatty acid and polyethylene glycol diester” refers to a diester derived from two fatty acid molecules and a polyethylene glycol molecule, represented by

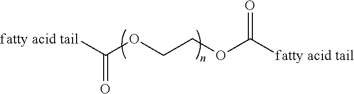

wherein n refers to the number of ethylene oxide units (—O—CH₂—CH₂—) per molecule.

The polyethylene glycol component of the fatty acid and polyethylene glycol esters and diesters may be defined by the average (e.g. mean) number of ethylene oxide units per molecule of polyethylene glycol. The polyethylene glycol component may be defined by its average molecular weight.

An average molecular weight may, for example, refer to a number average or weight average molecular weight. Average molecular weight may, for example, be measured using gel permeation chromatography.

The term “fatty acid and glycerol monoester” refers to an ester derived from a fatty acid molecule and a glycerol molecule, represented by

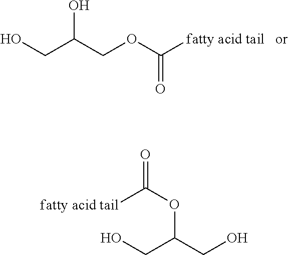

This can alternatively be referred to as a monoglyceride.

The term “fatty acid and glycerol diester” refers to a diester derived from two fatty acid molecules and a glycerol molecule, represented by

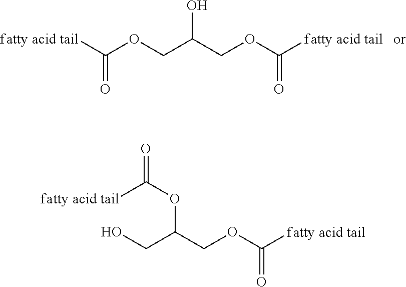

This can alternatively be referred to as a diglyceride.

The term “fatty acid and glycerol triester” refers to a triester derived from three fatty acid molecules and a glycerol molecule, represented by

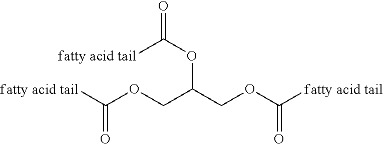

This can alternatively be referred to as a triglyceride.

The second component may be defined in terms of its fatty acid content. This includes the fatty acids in the fatty acid and polyethylene glycol monoesters and diesters, and, where present, the fatty acid and glycerol monoesters, diesters and triesters, as well any free fatty acid that may be present. The amount of each fatty acid present may be given as a percentage of the total fatty acid content in the second component. For example, this may be written as “the second component may comprise at least about 20% stearic acid relative to the total fatty acid content”. Throughout this disclosure, where the fatty acid present is defined in terms of percentage values relative to the total fatty acid content, the percentage may be determined by gas chromatography, for example, using the procedure provided in 2.4.22 of the European Pharmacopoeia 10.0, which is incorporated herein by reference. The procedure may be method A, method B, or preferably method C of 2.4.22 of the European Pharmacopoeia 10.0.

The second component may also be defined in terms of the percentage of polyethylene glycol monoesters and diesters, the percentage of glycerol monoesters, diesters and triesters, the percentage of free polyethylene glycol and/or the percentage of free glycerol present. Throughout this disclosure, where the second component is defined in terms of percentage values, the percentage may be w/w % relative to the total weight of the second component, v/v % relative to the total volume of the second component, or mol % relative to the total moles of the second component. Preferably, the percentage is w/w % relate to the total weight of the second component. The percentage of free glycerol present in the second component may be determined using the procedure provided in the “Lauroyl macrogolglycerides” monograph of the European Pharmacopoeia 10.0, which is incorporated herein by reference.

The term “drop point” refers to the temperature at which the first drop of a melting substance to be examined falls from a cup. The drop point may be determined using the procedure provided in 2.2.17 of the European Pharmacopoeia 9.6, which is incorporated herein by reference. The procedure may be method A of 2.2.17 or preferably method B (“automated method”) of 2.2.17 of the European Pharmacopoeia 9.6.

The term “hydrophile-lipophile balance” (HLB) is the measure of the degree to which a surfactant is hydrophilic or lipophilic. An HLB value can be a calculated value or a practical value. The calculated value may be determined using the method described in Griffin WC, “Calculation of HLB values of non-ionic surfactants”, Journal of the Society of Cosmetic Chemists, 5 (1654): 259 or in Davies JT, “A quantitative kinetic theory of emulsion type, I. Physical chemistry of the emulsifying agent”, Gas Liquid and Liquid/Liquid interface. Proceedings of the International Congress of the Surface Activity (1657): 426-438 (both of which are incorporated herein by reference). The practical value may be determined using the following emulsification method: The second component is formulated in a series of emulsions comprising a standard surfactive excipient (eg. Span 20 HLB=8.6 or Span 80 HLB=4.3 or Tween 80 HLB=15). The choice of standard surfactant depends on the calculated HLB of the second component. The emulsions are made with mineral oil (with a required HLB of 10) and coloured purified water. Mineral oil and purified water are added at 15 and 80% respectively. A series of emulsions are formulated with a ratio of second component to Span 20 or Span 80 or Tween 80 ranging from 0.5/4.5% to 4.5/0.5% to cover a range of HLB values. The emulsion which shows the highest stability is that in which the practical HLB of the mixture of surfactants is the closest to the required HLB of the oil. An equation is then applied to determine the practical HLB of the second component, using the ratios of the most stable emulsion:

$${HLB}_{required} = \frac{{m}_{A}}{{m}_{A} + {m}_{B}} \times {HLB}_{A} + \frac{{m}_{B}}{{m}_{A} + {m}_{B}} \times {HLB}_{B}$$

wherein A is the standard surfactive excipient and B is the second component.

The term “subject” refers to an animal, preferably a mammal, most preferably a human, who has been the object of treatment, observation or experiment.

The term “therapeutically effective amount” refers to an amount of an active compound or pharmaceutical agent which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease.

The term “therapeutically effective amount” may refer to the amount of a formulation of the present invention that, when administered to a subject, is effective to (1) at least partially alleviate, inhibit, prevent, and/or ameliorate a condition, or a disorder or a disease (i) mediated by MALT1; or (ii) associated with MALT1 activity; or (iii) characterized by activity (normal or abnormal) of MALT1; or (2) reduce or inhibit the activity of MALT1; or (3) reduce or inhibit the expression of MALT1; or (4) modify the protein levels of MALT1.

The term “MALT1-mediated” refers to any disease, syndrome, condition, or disorder that might occur in the absence of MALT1 but can occur in the presence of MALT1. Suitable examples of a disease, syndrome, condition, or disorder mediated by MALT1 include, but are not limited to, lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocytic leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

As used herein, the term “MALT1 inhibitor” refers to an agent that inhibits or reduces at least one condition, symptom, syndrome, disorder, and/or disease of MALT1.

As used herein, unless otherwise noted, the term “affect” or “affected” (when referring to a disease, syndrome, condition or disorder that is affected by the inhibition of MALT1) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physical parameter including those which may not be discernible by the patient. In a further embodiment, “treat”, “treating”, or “treatment” refers to modulating the disease, condition, syndrome or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome or disorder.

### Pharmaceutical Formulations

The invention provides a pharmaceutical formulation, comprising a first component and a second component;
wherein the first component is an active pharmaceutical ingredient (API) which is a compound as described herein, for example a compound of Formula (I) as described herein, for example a compound of Formula (I):

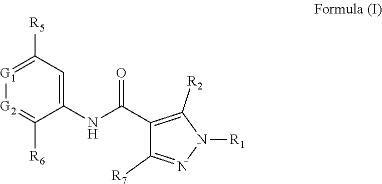

wherein
   R₁ is selected from the group consisting of
i) naphthalen-1-yl, optionally substituted with a fluoro or amino substituent;
   and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
   R₂ is selected from the group consisting of C₁₋₄alkyl, 1-methoxy-ethyl, difluoromethyl, fluoro, chloro, bromo, cyano, and trifluoromethyl;
   G₁ is N or C(R₄);
   G₂ is N or C(R₃); such that only one of G₁ and G₂ are N in any instance;
   R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
   R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy;
   and
xviii) morpholin-4-ylcarbonyl;
   R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
   or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
   R₆ is hydrogen, C₁₋₄alkyl, fluoro, 2-methoxy-ethoxy, chloro, cyano, or trifluoromethyl; and
   R₇ is hydrogen or fluoro;
or an enantiomer, diastereomer, solvate, or pharmaceutically acceptable salt form thereof, and
wherein the second component is a mixture comprising fatty acid and polyethylene glycol monoesters and diesters, and optionally, fatty acid and glycerol monoesters, diesters and triesters;
wherein the fatty acid component of the fatty acid and polyethylene glycol monoesters and diesters, and of the fatty acid and glycerol monoesters, diesters and triesters, when present, comprises one or more saturated fatty acids having at least eight carbons.

In particular, the invention provides a pharmaceutical formulation, comprising a first component and a second component; wherein the first component is an active pharmaceutical ingredient which is

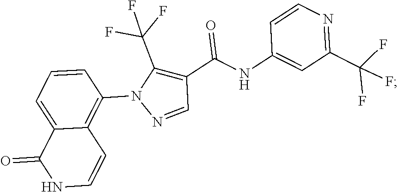 [SMILES: O=C(Nc1ccnc(C(F)(F)F)c1)c1cnn(-c2cccc3c(=O)[nH]ccc23)c1C(F)(F)F]

and
wherein the second component is a mixture comprising fatty acid and polyethylene glycol monoesters and diesters, and optionally, fatty acid and glycerol monoesters, diesters and triesters;
wherein the fatty acid component of the fatty acid and polyethylene glycol monoesters and diesters, and of the fatty acid and glycerol monoesters, diesters and triesters, when present, comprises one or more saturated fatty acids having at least eight carbons.

The pharmaceutical formulation of the invention may comprise at most about 50 w/w %, at most about 45 w/w %, at most about 40 w/w %, at most about 35 w/w %, or at most about 30 w/w % of the active pharmaceutical ingredient (API) relative to the total weight of the formulation. The pharmaceutical formulation may comprise at least about 0.1 w/w %, at least about 1 w/w %, at least about 5 w/w %, at least about 10 w/w %, or at least about 15 w/w % of the active pharmaceutical ingredient relative to the total weight of the formulation. The pharmaceutical formulation may comprise from about 0.1 w/w % to about 40 w/w %, from about 1 w/w % to about 30 w/w %, or from about 5 w/w % to about 25 w/w % of the active pharmaceutical ingredient relative to the total weight of the formulation. The formulation may comprise from about 12 w/w % to about 25 w/w % of the active pharmaceutical ingredient relative to the total weight of the formulation.

The pharmaceutical formulation of the invention may contain about 0.1 mg to about 3000 mg of the API, or any particular amount or range therein, in particular from about 1 mg to about 1000 mg of the API, or any particular amount or range therein, or, more particularly, from about 10 mg to about 500 mg of the API, or any particular amount or range therein, in a regimen of about 1 to about (4×) per day for an average (70 kg) human; although, it is apparent to one skilled in the art that the therapeutically effective amount for said API will vary as will the diseases, syndromes, conditions, and disorders being treated.

The pharmaceutical formulation of the invention comprises a second component which is a mixture comprising fatty acid and polyethylene glycol monoesters and diesters, and optionally, fatty acid and glycerol monoesters, diesters and triesters; wherein the fatty acid component of the fatty acid and polyethylene glycol monoesters and diesters, and of the fatty acid and glycerol monoesters, diesters and triesters, when present, comprises one or more saturated fatty acids having at least eight carbons. The fatty acid component may consist of or consist essentially of one or more saturated fatty acids having at least eight carbons.

The fatty acid component of the fatty acid and polyethylene glycol monoesters and diesters, and of the fatty acid and glycerol monoesters, diesters and triesters, when present, may comprise one or more saturated fatty acids having from 8 to 30 carbons, from 8 to 20 carbons, or from about 8 to 18 carbons. The aliphatic chain may be unbranched.

The fatty acid component of the fatty acid and polyethylene glycol monoesters and diesters may comprise stearic acid and optionally palmitic acid. The fatty acid component of the fatty acid and polyethylene glycol monoesters and diesters may comprise stearic acid and palmitic acid. The second component may be substantially free of fatty acid and glycerol monoesters, diesters and triesters. In the present context, substantially free means that the second component has less than about 10%, preferably less than about 5%, more preferably less than about 2%, even more preferably less than about 1%, even more preferably less than about 0.5% and even more preferably less than about 0.1% of fatty acid and glycerol monoesters, diesters and triesters.

The fatty acid component of the fatty acid and polyethylene glycol monoesters and diesters may comprise stearic acid and palmitic acid and, optionally, caprylic acid, capric acid, lauric acid and/or myristic acid. The second component may comprise at least about 20%, at least about 30%, at least about 35%, or at least about 40% stearic acid relative to the total fatty acid content. The second component may comprise at least about 20%, at least about 30%, at least about 35%, or at least about 40% palmitic acid relative to the total fatty acid content. The second component may comprise at least about 70%, at least about 80%, at least about 85%, or at least about 90% stearic and palmitic acid combined, relative to the total fatty acid content. The second component may comprise from about 40% to about 60% stearic acid and at least about 90% palmitic and stearic acid combined, relative to the total fatty acid content. The second component may comprise from about 90% to about 99% stearic acid and at least about 96% palmitic and stearic acid combined, relative to the total fatty acid content.

The second component may comprise a mixture of fatty acid and polyethylene glycol monoesters and diesters and fatty acid and glycerol monoesters, diesters and triesters.

The fatty acid component of the fatty acid and polyethylene glycol monoesters and diesters and the fatty acid and glycerol monoesters, diesters and triesters may comprise stearic acid and optionally palmitic acid. The fatty acid component of the fatty acid and polyethylene glycol monoesters and diesters and the fatty acid and glycerol monoesters, diesters and triesters may comprise stearic acid and palmitic acid. The second component may comprise at least about 20%, at least about 30%, at least about 35%, at least about 40% or at least about 45% stearic acid relative to the total fatty acid content. The second component may comprise at least about 20%, at least about 30%, at least about 35% or at least about 40% palmitic acid relative to the total fatty acid content. The second component may comprise at least about 70%, at least about 80%, at least about 85% or at least about 90% stearic and palmitic acid combined, relative to the total fatty acid content. The fatty acid component of the fatty acid and polyethylene glycol monoesters and diesters and the fatty acid and glycerol monoesters, diesters and triesters may comprise stearic acid, palmitic acid, optionally lauric acid and optionally myristic acid. The second component may comprise at most about 5% lauric acid, at most about 5% myristic acid, from about 40% to about 50% palmitic acid and from about 48% to about 58% stearic acid relative to the total fatty acid content. The second component may comprise at least about 90% of stearic and palmitic acid combined, relative to the total fatty acid content. The fatty acid component of the fatty acid and polyethylene glycol monoesters and diesters and the fatty acid and glycerol monoesters, diesters and triesters may comprise stearic acid, palmitic acid, optionally lauric acid, optionally myristic acid, optionally caprylic acid and optionally capric acid. The second component may comprise at most about 3% caprylic acid, at most about 3% capric acid, at most about 5% lauric acid, at most about 5% myristic acid, from about 40% to about 50% palmitic acid and from about 48% to about 58% stearic acid relative to the total fatty acid content. The second component may comprise at least about 90% palmitic and stearic acid combined, relative to the total fatty acid content.

The fatty acid component of the fatty acid and polyethylene glycol monoesters and diesters and the fatty acid and glycerol monoesters, diesters and triesters may comprise lauric acid. The second component may comprise at least about 10%, at least about 20%, at least about 25% or at least about 30% lauric acid relative to the total fatty acid content. The fatty acid component of the fatty acid and polyethylene glycol monoesters and diesters and the fatty acid and glycerol monoesters, diesters and triesters may comprise lauric acid, palmitic acid, stearic acid, myristic acid, optionally caprylic acid and optionally capric acid. The second component may comprise at most about 15% caprylic acid, at most about 12% capric acid, from about 30% to about 50% lauric acid, from about 5% to about 25% myristic acid, from about 4% to about 25% palmitic acid and from about 5% to about 35% stearic acid relative to the total fatty acid content.

The second component may comprise at least about 5%, at least about 10%, at least about 15% or at least about 20% glycerol mono-, di-, and triesters. The second component may comprise at least about 50%, at least about 60%, at least about 65% or at least about 70% polyethylene glycol mono- and diesters. The second component may comprise from about 10% to about 30% glycerol mono-, di-, and triesters. The second component may comprise from about 15% to about 25% glycerol mono-, di-, and triesters. The second component may comprise about 20% glycerol mono-, di-, and triesters. The second component may comprise from about 60% to about 80% polyethylene glycol mono- and diesters. The second component may comprise from about 65% to about 75% polyethylene glycol mono- and diesters. The second component may comprise about 72% polyethylene glycol mono- and diesters. The second component may comprise at least about 90% ester content.

The second component may comprise free polyethylene glycol. The second component may comprise at most about 8% free polyethylene glycol. The second component, when fatty acid and glycerol monoesters, diesters and triesters are present, may comprise free glycerol. The second component may comprise at most about 3% free glycerol. The second component may comprise free fatty acid.

The second component may have a drop point of at least about 30° C. The second component may have a drop point of from about 30° C. to about 70° C., from about 35° C. to about 70° C., from about 35° C. to about 65° C., from about 40° C. to about 60° C., or from about 40° C. to about 55° C. The second component may have a drop point of from about 40° C. to about 55° C.

The second component can also be characterised by its “melting point”. The second component may have a melting point of at least about 30° C. The second component may have a melting point of from about 30° C. to about 70° C., from about 35° C. to about 70° C., from about 35° C. to about 65° C., from about 40° C. to about 60° C., or from about 40° C. to about 55° C. The second component may have a melting point of from about 40° C. to about 55° C. In a particular embodiment, the pharmaceutical formulation of the invention comprises second component having an upper limit of the melting point of at least about 30° C. The second component may have an upper limit of the melting point of from about 30° C. to about 70° C., from about 35° C. to about 70° C., from about 35° C. to about 65° C., from about 40° C. to about 60° C., or from about 40° C. to about 55° C. The second component may have an upper limit of the melting point of from about 40° C. to about 55° C.

As an example, polyoxyl-32-stearate type I, described below, has a melting point of 46-50° C., which means that the upper limit of the melting point is 50° C. The melting point may be determined using the procedure provided in 2.2.15 of the European Pharmacopoeia 10.0, which is incorporated herein by reference.

The above melting points of the second component can alternatively be referred to as “freezing point”. The above melting point values and ranges therefore also provide equivalent freezing point values and ranges. The second component may also be characterised by freezing point. The freezing point may be determined using the procedure provided in 2.2.18 of the European Pharmacopoeia 10.0, which is incorporated herein by reference.

The second component may have a calculated hydrophile-lipophile balance (HLB) of from about 8 to about 18. The second component may have a calculated HLB of from about 10 to about 18. The second component may have a calculated HLB of from about 12 to about 17. The second component may have a calculated HLB of about 13, about 14 or about 16. The second component may have a practical HLB of from about 8 to about 18. The second component may have a practical HLB of from about 10 to about 14. The second component may have a practical HLB of about 11 or about 12.

The polyethylene glycol of the fatty acid and polyethylene glycol monoesters and diesters may be characterised by its average molecular weight or by the average number of ethylene oxide units per molecule of polyethylene glycol.

The polyethylene glycol (PEG) of the fatty acid and polyethylene glycol monoesters and diesters may have an average molecular weight of at least about 200 g/mol or at least about 250 g/mol. The polyethylene glycol may have an average molecular weight of from about 250 to about 20000 g/mol, from about 250 to about 10000 g/mol, from about 250 to 5000 g/mol, or from about 1000 g/mol to about 2000 g/mol. The polyethylene glycol may have an average molecular weight of at least about 900 g/mol, or at least about 1000 g/mol. The polyethylene glycol may have an average molecular weight of at from about 1300 g/mol to about 1700 g/mol. The polyethylene glycol may have an average molecular weight of from about 1400 g/mol to about 1600 g/mol. The polyethylene glycol may be a PEG grade selected from PEG300, PEG400, PEG600, PEG800, PEG1000, PEG1400, PEG1450, PEG1500, PEG1540, PEG2000, PEG3000, PEG3350, PEG3400, PEG4000, PEG4600, PEG5500, PEG6000, PEG8000, PEG9000, PEG10000, PEG12000 and PEG20000. The polyethylene glycol may be selected from PEG1500, PEG2000 and PEG3000. The polyethylene glycol may be selected from PEG1400, PEG1450, PEG1500, and PEG1540. The polyethylene glycol may comprise a mixture of two or more PEG grades.

Various PEG grades are commercially available. Characterisation of various PEG grades is, for example, provided in the European Pharmacopoeia 10.0 (“Macrogols”, page 3145-3147, incorporated herein by reference).

The PEG grades disclosed herein may refer to polyethylene glycols with average molecular weights within a range corresponding to the specified grade as set out in the European Pharmacopoeia 10.0. The range of average molecular weights may be at most about +/−10% of the specified grade. For example, PEG1000 may be a polyethylene glycol with an average molecular weight of 950-1050 g/mol. PEG1450 may be a polyethylene glycol with an average molecular weight of 1305-1595 g/mol. PEG1500 may be a polyethylene glycol with an average molecular weight of 1400-1600 g/mol. PEG1540 may be a polyethylene glycol with an average molecular weight of 1386-1694 g/mol. PEG2000 may be a polyethylene glycol with an average molecular weight of 1800-2200 g/mol. PEG3000 may be a polyethylene glycol with an average molecular weight of 2700-3300 g/mol. PEG4000 may be a polyethylene glycol with an average molecular weight of 3700-4400 g/mol.

The average molecular weight may be determined using the procedure provided in the US Pharmacopoeia Official Monographs, page information USP42-NF37-5882 (“Polyethylene Glycol, Assay, Average Molecular Weight”) which is incorporated herein by reference.

The polyethylene glycol (PEG) may have an average of at least 5 ethylene oxide units per molecule. The polyethylene glycol (PEG) may have an average of from 6 to 100 ethylene oxide units per molecule, from 10 to 50 ethylene oxide units per molecule, or from 20 to 40 ethylene oxide units per molecule. The polyethylene glycol (PEG) may have an average of from 30 to 35 ethylene oxide units per molecule.

The polyethylene glycol may be a PEG grade defined by the average number of ethylene oxide units per molecule. The polyethylene glycol may be a PEG grade selected from PEG-10, PEG-15, PEG-20, PEG-25, PEG-30, PEG-32, PEG-33, PEG-35, PEG-40, PEG-45, PEG-50, PEG-55, PEG60, PEG-75, or PEG-90. The polyethylene glycol may be PEG-32.

Names and abbreviations for polyethylene glycol include but are not limited to poly(ethylene oxide), PEG and macrogol. Macrogol is the international non-proprietary name for polyethylene glycol used in medicine.

The second component may be polyoxyl stearate. Polyoxyl stearate comprises a mixture of stearic acid and polyethylene glycol monoesters and diesters and optionally palmitic acid and polyethylene glycol monoesters and diesters. Polyoxyl stearate may comprise a mixture of stearic acid and polyethylene glycol monoesters and diesters and palmitic acid and polyethylene glycol monoesters and diesters. Polyoxyl stearate may contain an average polymer length of equivalent to 6-100 ethylene oxide units per molecule of polyethylene glycol. Polyoxyl stearate may contain free polyethylene glycol. Polyoxyl stearate may be as defined in the USP-NF (for example, in USP42-NF37-5904, which is incorporated herein by reference). Polyoxyl stearate may alternatively be referred to as polyethylene glycol stearate, macrogol stearate, or poly(oxy-1,2-ethanediyl), α-hydro-o-hydroxyoctadecanoate. Polyoxyl stearate may be as defined in the European Pharmacopeia 10.0 (“Macrogol stearate”, page 3142, incorporated herein by reference). Polyoxyl stearate may comprise from about 40% to about 60% stearic acid and at least about 90% palmitic and stearic acid combined, relative to the total fatty acid content. This can be referred to as polyoxyl stearate type I (for example, as defined in USP42-NF37-5904). Polyoxyl stearate may comprise from about 90% to about 99% stearic acid and at least about 96% palmitic and stearic acid combined, relative to the total fatty acid content. This can be referred to as polyoxyl stearate type II (for example, as defined in USP42-NF37-5904). The polyoxyl stearate may have an average polymer length of 32 ethylene oxide units per molecule of polyethylene glycol. This may be referred to as polyoxyl-32 stearate or, alternatively, as PEG-32 stearate. In particular, the polyoxyl-32 stearate may be polyoxyl-32 stearate type I. Polyoxyl stearate may have a drop point of from about 40° C. to about 55° C., for example from about 46° C. to about 50° C. Polyoxyl-32 stearate type I may have a drop point of about 46° C. to about 50° C. An example of commercially available polyoxyl-32 stearate type I is Gelucire® 48/16.

The second component may be stearoyl polyoxylglycerides. Stearoyl polyoxylglycerides comprises a mixture of stearic and palmitic acid and polyethylene glycol monoesters and diesters, and stearic and palmitic acid and glycerol monoesters, diesters and triesters. The polyethylene glycol component of the stearoyl polyethylene glycol esters may have an average molecular weight of from about 300 to about 4000 g/mol. Stearoyl polyoxylglycerides may contain free polyethylene glycol. Stearoyl polyoxylglycerides may contain free glycerol. Stearoyl polyoxylglycerides may comprise polyethylene glycol monoesters and diesters and glycerol monoesters, diesters and triesters of stearic acid, palmitic acid, optionally lauric acid, optionally myristic acid, optionally caprylic acid and optionally capric acid. Stearoyl polyoxylglycerides may comprise at most about 5% lauric acid, at most about 5% myristic acid, from about 40% to about 50% palmitic acid and from about 48% to about 58% stearic acid relative to the total fatty acid content. Stearoyl polyoxylglycerides may comprise at most about 3% caprylic acid, at most about 3% capric acid, at most about 5% lauric acid, at most about 5% myristic acid, from about 40% to about 50% palmitic acid and from about 48% to about 58% stearic acid relative to the total fatty acid content. Stearoyl polyoxylglycerides may comprise at least about 90% palmitic and stearic acid combined, relative to the total fatty acid content. Stearoyl polyoxylglycerides may be as defined in the USP-NF (for example, in USP42-NF37-6010, which is incorporated herein by reference). Stearoyl polyoxylglycerides may alternatively be referred to as PEG glyceryl stearate or stearoyl macrogolglycerides. Stearoyl polyoxylglycerides may be as defined in the European Pharmacopeia 5.0 (“Stearoyl Macrogolglycerides”, page 2491-2492, incorporated herein by reference). The second component may be a stearoyl polyoxylglycerides wherein the polyethylene glycol has an average polymer length of 32 ethylene oxide units per molecule of polyethylene glycol. This may be referred to as stearoyl polyoxyl-32 glycerides or, alternatively, as stearoyl PEG-32 glycerides, stearoyl macrogol-32 glycerides, or hydrogenated palm oil PEG-32 esters. Stearoyl polyoxyl-32 glycerides may comprise at least about 5%, at least about 10%, at least about 15% or at least about 20% glycerol mono-, di-, and triesters. Stearoyl polyoxyl-32 glycerides may comprise at least about 50%, at least about 60%, at least about 65% or at least about 70% PEG-32 mono- and diesters. Stearoyl polyoxyl-32 glycerides may comprise from about 10% to about 30% glycerol mono-, di-, and triesters. Stearoyl polyoxyl-32 glycerides may comprise from about 15% to about 25% glycerol mono-, di-, and triesters. Stearoyl polyoxyl-32 glycerides may comprise from about 60% to about 80% polyethylene glycol mono- and diesters. Stearoyl polyoxyl-32 glycerides may comprise from about 65% to about 75% polyethylene glycol mono- and diesters. Stearoyl polyoxyl-32 glycerides may comprise at most about 3% free glycerol. Stearoyl polyoxylglycerides may have a drop point of from about 40° C. to about 55° C., for example from about 46° C. to about 51° C. Stearoyl polyoxyl-32 glycerides may have a drop point of from about 46° C. to about 51° C. An example of commercially available stearoyl polyoxyl-32 glycerides is Gelucire® 50/13. Another example of commercially available stearoyl polyoxyl-32 glycerides is Acconon® C-50 EP/NF.

The second component may be lauroyl polyoxylglycerides. Lauroyl polyoxylglycerides comprises a mixture of lauric acid and polyethylene glycol monoesters and diesters, and lauric acid and glycerol monoesters, diesters and triesters. The polyethylene glycol component of the lauroyl polyethylene glycol esters may have an average molecular weight of from about 300 to about 4000 g/mol or from about 300 to about 1500 g/mol. Lauroyl polyoxylglycerides may contain free polyethylene glycol. Lauroyl polyoxylglycerides may contain free glycerol. Lauroyl polyoxylglycerides may comprise polyethylene glycol monoesters and diesters and glycerol monoesters, diesters and triesters of lauric acid, myristic acid, palmitic acid, stearic acid, optionally caprylic acid, and optionally capric acid. Lauroyl polyoxylglycerides may comprise at most about 15% caprylic acid, at most about 12% capric acid, from about 30% to about 50% lauric acid, from about 5% to about 25% myristic acid, from about 4% to about 25% palmitic acid and from about 5% to about 35% stearic acid relative to the total fatty acid content. Lauroyl polyoxylglycerides may be as defined in the USP-NF (for example, in USP42-NF37-5799, which is incorporated herein by reference). Lauroyl polyoxylglycerides may alternatively be referred to as PEG glyceryl laurate or lauroyl macrogolglycerides. Lauroyl polyoxylglycerides may be defined as in the European Pharmacopeia 10.0 (“Lauroyl Macrogolglycerides”, page 3068-3069, incorporated herein by reference). The second component may be lauroyl polyoxylglycerides wherein the polyethylene glycol has an average polymer length of 32 ethylene oxide units per molecule of polyethylene glycol. This may be referred to as lauroyl polyoxyl-32 glycerides or, alternatively, as lauroyl PEG-32 glycerides, lauroyl macrogol-32 glycerides, or hydrogenated coconut oil PEG-32 esters. Lauroyl polyoxyl-32 glycerides may comprise at least about 5%, at least about 10%, at least about 15%, or at least about 20% glycerol mono-, di-, and triesters. Lauroyl polyoxyl-32 glycerides may comprise at least about 50%, at least about 60%, at least about 65%, or at least about 70% of PEG-32 mono- and diesters. Lauroyl polyoxyl-32 glycerides may comprise from about 10% to about 30% glycerol mono-, di-, and triesters. Lauroyl polyoxyl-32 glycerides may comprise from about 15% to about 25% glycerol mono-, di-, and triesters. Lauroyl polyoxyl-32 glycerides may comprise about 20% glycerol mono-, di-, and triesters. Lauroyl polyoxyl-32 glycerides may comprise from about 60% to about 80% polyethylene glycol mono- and diesters. Lauroyl polyoxyl-32 glycerides may comprise from about 65% to about 75% polyethylene glycol mono- and diesters. Lauroyl polyoxyl-32 glycerides may comprise about 72% polyethylene glycol mono- and diesters. Lauroyl polyoxyl-32 glycerides may comprise about 20% glycerol mono-, di-, and triesters, about 72% of PEG-32 mono- and diesters and about 8% of free polyethylene glycol. Lauroyl polyoxyl-32 glycerides may comprise at most about 3% of free glycerol.

Lauroyl polyoxylglycerides, may have a drop point of from about 35° C. to about 55° C. or from about 40° C. to about 55° C., for example from about 42° C. to about 47.5° C. Lauroyl polyoxyl-32 glycerides may have a drop point of from about 42° C. to about 47.5° C. An example of commercially available lauroyl polyoxyl-32 glycerides is Gelucire® 44/14. Another example of commercially available stearoyl polyoxyl-32 glycerides is Acconon® C-44 EP/NF.

The pharmaceutical formulation of the invention may comprise at least about 20 w/w %, at least about 30 w/w %, at least about 40 w/w %, at least about 50 w/w %, at least about 60 w/w %, or at least about 65 w/w % of the second component relative to the total weight of the formulation. The pharmaceutical formulation may comprise from about 70 w/w % to about 95 w/w %, from about 70 w/w % to about 90 w/w %, or from about 75 w/w % to about 90 w/w % of the second component relative to the total weight of the formulation.

A formulation according to the invention, having a second component comprising a mixture of fatty acid and polyethylene glycol monoesters and diesters, and fatty acid and glycerol monoesters, diesters and triesters, may be referred to as a self-emulsifying drug delivery system (SEDDS), a self-microemulsifying drug delivery system (SMEDDS) or as a type III formulation of the Lipid Formulation Classification System (LFCS) (Eur. J. Pharm. Sci, 2006, 29(3-4), 278-287). Without being bound by theory, upon contact with aqueous/digestive media, the formulation may spontaneously form a fine dispersion and the different fractions may self-assemble based on their affinity for water: polyethylene glycol is water-soluble; polyethylene glycol mono- and diesters and monoglycerides are amphiphilic; and di- and triglycerides are hydrophobic. When administered to a patient the glycerides fraction may be digested in the stomach to monoglycerides and free fatty acids and the polyethylene glycol esters fraction may be partially digested in the intestines. The amphiphilic compounds may associate with the digested compounds and self-assemble into colloidal structures (e.g. multi-lamellar, vesicles, mixed micelles and micelles). These structures have variable solubilizing capacities and contribute to maintaining the drug in solubilized state throughout the on-going digestion process. The fatty acids, monoglycerides and API may partition out of the mixed micelles and be absorbed in the intestine.

A formulation according to the invention, having a second component comprising a mixture of fatty acid and polyethylene glycol monoesters and diesters and substantially free of fatty acid and glycerol monoesters, diesters and triesters, may be referred to as a micellar drug delivery system or as a type IV formulation of the Lipid Formulation Classification System (LFCS). Type IV formulations contain hydrophilic components and may form micellar solutions on contact with aqueous media. Without being bound by theory, during the initial dispersion phase polyethylene glycol chains may hydrate forming viscous liquid crystalline mesophases which erode to form a micellar solution. The solubility of the active ingredient in the aqueous phase gradually increases due to the relatively slow hydration and micellization process. The risk of drug precipitation can be reduced by avoiding a sudden increase in drug solubility. The second component may assist with maintaining the active ingredient in a solubilized state within the micellar solution. During digestion the polyethylene glycol diester component may provide a “reservoir” of surfactant which is digested to monoesters (a stronger surfactant) which replenishes the micellar system maintaining the drug in a solubilized state.

As such, a formulation according to the invention is able to improve solubility, dissolution, stability and bioavailability of the API.

A formulation according to the invention may be supersaturatable. For example, the therapeutic dose in a formulation of the invention may exceed 100% API saturation at storage conditions. The solubility of the API above the drop point of the second component will be sufficient with respect to target strength of the formulation. However, the solubility of the molecule at room temperature or at 5° C. could be lower than the desired dose. The molecule, in such state, may be in a super-saturated state which may be kinetically stable at room temperature for the entire shelf life of the formulation.

The pharmaceutical formulation of the invention optionally comprises an antioxidant. The antioxidant may be selected from tocopherol (vitamin E), thiodipropionic acid, lipoic acid, hydroquinone, phytic acid, monothioglycerol, sodium thioglycolate, thioglycol, vitamin E acetate, beta carotene, butylated hydroxyanisole (BHA), butylated hydroxytoluene (BHT), cysteine, cysteine hydrochloride, propyl gallate (PG), sodium metabisulfite, ascorbyl palmitate, ascorbyl stearate, potassium metabisulfite, disodium EDTA (ethylenediamine tetraacetic acid; also known as disodium edentate), EDTA, erythorbic acid, ethoxyquin, glutathione, gum guaiac, lecithin, TBHQ (tert butyl hydroxyquinone), tartaric acid, citric acid, citric acid monohydrate, methane sulfonic acid, methionine, sodium thiosulfate, sodium sulphite, and a combination thereof.

The antioxidant may be selected from tocopherol (vitamin E), lipoic acid, hydroquinone, monothioglycerol, thioglycol, beta carotene, butylated hydroxyanisole (BHA), butylated hydroxytoluene (BHT), propyl gallate (PG), ascorbyl palmitate, ascorbyl stearate, ethoxyquin, TBHQ (tert butyl hydroxyquinone), and a combination thereof. The antioxidant may be tocopherol (vitamin E) or propyl gallate. The antioxidant may be tocopherol (vitamin E). The antioxidant may be propyl gallate. In a particular embodiment the tocopherol (vitamin E) is all-rac-alpha tocopherol. All-rac-alpha tocopherol may alternatively be referred to as DL-alpha-tocopherol.

The antioxidant may be all-rac-alpha tocopherol.

The pharmaceutical formulation of the invention may comprise from about 0.001 w/w % to about 2 w/w % of antioxidant relative to the total weight of the formulation. The pharmaceutical formulation may comprise from about 0.001 w/w % to about 1 w/w % of antioxidant relative to the total weight of the formulation. The pharmaceutical formulation may comprise from about 0.01 w/w % to about 2 w/w % of antioxidant relative to the total weight of the formulation. The pharmaceutical formulation may comprise from about 0.01 w/w % to about 1 w/w % of antioxidant relative to the total weight of the formulation. The pharmaceutical formulation may comprise from about 0.01 w/w % to about 0.5 w/w % of antioxidant relative to the total weight of the formulation. The pharmaceutical formulation may comprise about 0.01 w/w % or about 0.1 w/w % of antioxidant.

The pharmaceutical formulation of the invention optionally comprises a crystallisation rate inhibitor. The term “crystallisation rate inhibitor” refers to an excipient, for example a polymeric excipient, that is added to the formulation with the aim of inhibiting crystallisation of an API when the formulation is administered to a subject. A crystallisation rate inhibitor may be used to improve the bioavailability of an API where the crystalline form is typically significantly lower in comparison to the amorphous/dissolved state. The crystallisation rate inhibitor may be referred to as a crystallisation inhibitor or a stabilizer.

In an embodiment, the crystallisation rate inhibitor is selected from polyvinylpyrrolidone (PVP), a polyvinylpyrrolidone-vinyl acetate copolymer (PVPVA), a poly(meth)acrylate polymer (e.g. methacrylic acid-methyl methacrylate copolymer), a cyclodextrin or a cyclodextrin derivative (e.g. (2-hydroxypropyl)-β-cyclodextrin (HPBCD)), hydroxypropylcellulose, hydroxyethylcellulose, methylcellulose, hydroxypropyl methylcellulose (HPMC), hydroxypropyl methylcellulose acetate succinate (HPMCAS), a polyethylene glycol-polyvinyl acetate-polyvinyl caprolactame graft copolymer, poly(vinyl alcohol), a poloxamer (e.g. poloxamer 188, 338, or 407), and combinations thereof.

In an embodiment, the crystallisation rate inhibitor is selected from hydroxypropyl methylcellulose (HPMC), hydroxypropyl methylcellulose acetate succinate (HPMCAS), a polyethylene glycol-polyvinyl acetate-polyvinyl caprolactame graft copolymer, polyvinylpyrrolidone (PVP) and a polyvinylpyrrolidone-vinyl acetate copolymer (PVPVA), and a combination thereof. In a further embodiment, the crystallisation rate inhibitor is selected from hydroxypropyl methylcellulose (HPMC) and polyvinylpyrrolidone-vinyl acetate copolymer (PVPVA). The PVPVA may be a copolymer of 1-vinyl-2-pyrrolidone and vinyl acetate in a ratio of 6:4 by mass (PVPVA64).

Names and abbreviations for polyvinylpyrrolidone-vinyl acetate copolymer include, but are not limited to, PVPVA, PVP-Vac-copolymer, and poly(1-vinylpyrrolidone-co-vinyl-acetate).

Names and abbreviations for a copolymer of 1-vinyl-2-pyrrolidone and vinyl acetate in a ratio of 6:4 by mass (PVPVA64) include, but are not limited to, copolyvidone, copovidum, and copovidone. Examples of commercially available PVPVA64 are Kollidon® VA64, Kollidon® VA64 Fine, Luviskol VA64®, and Plasdone S-630@.

Names and abbreviations for polyvinylpyrrolidone include, but are not limited to, PVP, povidone and crospovidone. Crospovidone is a crosslinked homopolymer of vinyl pyrrolidone. An example of commercially available PVP is Plasdone® K-12.

Examples of commercially available poly(meth)acrylate polymers are Eudragit® polymers. Eudragit® polymers include amino alkyl methacrylate copolymers, methacrylic acid copolymers, methacrylic ester copolymers, and ammonioalkyl methacrylate copolymers. For example, Eudragit® L 100-55 is a copolymer of ethyl acrylate and methacrylic acid.

An example of a commercially available HPBCD is Cavasol®.

An example of a commercially available polyethylene glycol-polyvinyl acetate-polyvinylcaprolactame-based graft copolymer is Soluplus®.

Names and abbreviations for hydroxypropylmethylcellulose (HPMC) include, but are not limited to, hypromellose.

An example of a commercially available HPMC is Methocel®. An example of a commercially available HPMCAS is Affinisol™.

An example of a commercially available hydroxylpropylcellulose is Klucel™ ELF PHARM. An example of a commercially available hydroxyethylcellulose is Natrosol™ 250L PHARM. An example of a commercially available poly(vinyl alcohol) is Mowiol® 8-88.

Poloxamers are triblock copolymers based on poly(ethylene oxide) and poly(propylene oxide). Examples of commercially available poloxamers are Pluronic® polymers.

The crystallisation rate inhibitor may be soluble in the second component or may form a suspension in the second component.

The solid dosage form may be a capsule which has the role of the crystallisation rate inhibitor. For example, the capsule might be a hydroxypropyl methylcellulose (HPMC) capsule.

The pharmaceutical formulation of the invention may comprise at most about 20 w/w % of the crystallisation rate inhibitor relative to the total weight of the formulation. The pharmaceutical formulation may comprise at least about 0.05 w/w % or at least about 0.1 w/w % of the crystallisation rate inhibitor relative to the total weight of the formulation. The pharmaceutical formulation may comprise from about 0.5 w/w % to about 15 w/w % or from about 0.5 w/w % to about 10 w/w % of the crystallisation rate inhibitor relative to the total weight of the formulation. The pharmaceutical formulation may comprise about 0.5 w/w %, about 1 w/w %, or about 5 w/w % of the crystallisation rate inhibitor relative to the total weight of the formulation.

Crystallisation inhibition may be useful in solid dosage forms, in particular those containing formulations of APIs, the absorption of which is solubility and/or dissolution rate limited, such as APIs belonging to BCS class II or IV. Without being bound to any theory, when a solid dosage form containing a second component described herein is administered, the second component may disperse (and be partially digested) in the aqueous environment in the gastrointestinal tract, eventually resulting in an API solvent shift from the second component to water. If the API is poorly soluble in water, this may lead to a high supersaturation of the API in the aqueous environment, resulting in precipitation. The presence of a crystallisation rate inhibitor may lead to the API precipitating out of solution as an amorphous form rather than a crystalline form. Amorphous forms may be resolubilised more quickly than crystalline forms, thus resulting in faster absorption of the API into the blood. Crystallisation rate inhibitors may therefore improve the absorption and hence improve oral bioavailability of APIs.

The pharmaceutical formulation of the invention may further comprise one or more pharmaceutically acceptable excipients, as described in more detail herein. Pharmaceutically acceptable excipients include, but are not limited to, disintegrants, binders, diluents, lubricants, stabilizers, osmotic agents, colorants, plasticizers, coatings and the like.

More particularly, suitable pharmaceutical excipients comprise one or more of the following: (i) diluents such as lactose, mannitol, microcrystalline cellulose, dicalcium phosphate, maltodextrin, starch and the like; (ii) binders such as polyvinylpyrrolidone (such as povidone), methylcellulose, hydroxypropyl cellulose, hydroxypropyl methylcellulose (such as METHOCEL® E-5), and the like; (iii) disintegrants such as sodium starch glycolate, croscarmellose sodium, crospovidone, L-HPC (low substituted hydroxypropylcellulose), pregelatinized starch, maize starch and the like; (iv) wetting agents such as surfactants, such as sodium lauryl stearate, docusate sodium, polysorbate 20, polysorbate 80 and the like; (v) lubricants such as magnesium stearate, sodium stearyl fumarate, stearic acid, talc, and the like; (vi) flow promoters or glidants such as colloidal silicon dioxide, talc and the like; and other excipients known to be useful in the preparation of pharmaceutical formulations; (vii) stabilizers such as myristic acid, palmitic acid, stearic acid, cetyl alcohol, cetostearyl alcohol, stearylalcohol, glyceryl distearate, glycerol monostearate, glyceryl dibehenate, hard fat or any combination thereof. Additional suitable pharmaceutical excipients and their properties may be found in texts such as Handbook of Pharmaceutical Excipients, Edited by R. C. Rowe, P. J. Sheskey & P. J. Weller, Sixth Edition (Published by Pharmaceutical Press, a Division of Royal Pharmaceutical Society of Great Britain).

Fillers or diluents for use in the pharmaceutical formulations of the present invention include fillers or diluents typically used in the formulation of pharmaceuticals. Examples of fillers or diluents for use in accordance with the present invention include, but are not limited to, sugars such as lactose, dextrose, glucose, sucrose, cellulose, starches and carbohydrate derivatives, polysaccharides (including dextrates and maltodextrin), polyols (including mannitol, xylitol, and sorbitol), cyclodextrins, calcium carbonates, magnesium carbonates, microcrystalline cellulose, combinations thereof, and the like. In certain preferred embodiments the filler or diluent is lactose, microcrystalline cellulose, or combination thereof. Several types of microcrystalline cellulose are suitable for use in the formulations described herein, for example, microcrystalline cellulose selected from the group consisting of Avicel® types: PH101, PH102, PH103, PH105, PH 112, PH113, PH200, PH301, and other types of microcrystalline cellulose, such as silicified microcrystalline cellulose. Several types of lactose are suitable for use in the formulations described herein, for example, lactose selected from the group consisting of anhydrous lactose, lactose monohydrate, lactose fast flo, directly compressible anhydrous lactose, and modified lactose monohydrate.

Binders for use in the pharmaceutical formulations of the present invention include binders commonly used in the formulation of pharmaceuticals. Examples of binders for use in accordance with the present invention include but are not limited to cellulose derivatives (including hydroxypropyl cellulose, hydroxypropyl methylcellulose, methylcellulose, and sodium carboxymethyl cellulose), glycol, sucrose, dextrose, corn syrup, polysaccharides (including acacia, targacanth, guar, alginates and starch), corn starch, pregelatinized starch, modified corn starch, gelatin, polyvinylpyrrolidone, polyethyleneglycol, combinations thereof and the like.

Disintegrants for use in the pharmaceutical formulations of the present invention include disintegrants commonly used in the formulation of pharmaceuticals. Examples of disintegrants for use in accordance with the present invention include but are not limited to starches, and crosslinked starches, celluloses and polymers, combinations thereof and the like. Representative disintegrants include microcrystalline cellulose, croscarmellose sodium, alginic acid, sodium alginate, crosprovidone, cellulose, agar and related gums, sodium starch glycolate, corn starch, potato starch, sodiumstarch glycolate, Veegum HV, methylcellulose, L-HPC (low substituted hydroxypropylcellulose), agar, bentonite, sodium carboxymethylcellulose, calcium carboxymethylcellulose, carboxymethylcellulose, alginic acid, guar gum, maize starch, pregelatinized starch, combinations thereof, and the like.

Lubricants, glidants or anti-tacking agents for use in the pharmaceutical formulations of the present invention include lubricants, glidants and anti-tacking agents commonly used in the formulation of pharmaceuticals. Examples for use in accordance with the present invention include but are not limited to magnesium carbonate, magnesium laurylsulphate, calcium silicate, talc, fumed silicon dioxide, combinations thereof, and the like. Other useful lubricants include but are not limited to magnesium stearate, calcium stearate, stearic acid, sodium stearyl fumarate, sodium lauryl sulphate, magnesium lauryl sulphate, sodium benzoate, colloidal silicon dioxide, magnesium aluminometasillicate (such as Neusilin®), magnesium oxide, magnesium silicate, mineral oil, hydrogenated vegetable oils, waxes, glyceryl behenate, and combinations thereof, and the like.

Surfactants for use in the pharmaceutical formulations of the present invention include surfactants commonly used in the formulation of pharmaceuticals. Examples of surfactants for use in accordance with the present invention include but are not limited to zwitterionic, ionic- and nonionic surfactants or wetting agents commonly used in the formulation of pharmaceuticals, such as ethoxylated castor oil, polyglycolyzed glycerides, acetylated monoglycerides, sorbitan fatty acid esters, poloxamers (e.g. Pluronic®), polyethylene glycol (15)-hydroxystearate (e.g. Solutol®), polyoxyethylene sorbitan fatty acid esters, polyoxyethylene derivatives, monoglycerides or ethoxylated derivatives thereof, diglycerides or polyoxyethylene derivatives thereof, dioctyl sulfosuccinate sodium salt (sodium docusate), sodium laurylsulfate (SLS), cholic acid or derivatives thereof, lecithins, phospholipids, combinations thereof, and the like. Non-ionic surfactants may have an HLB (hydrophile-lipophile balance) value higher than 10.

The pharmaceutical formulations disclosed herein can further comprise one or more flow regulators (or glidants). Flow regulators may be present in powders or granules and are admixed in order to increase their flowability of the formulation during manufacture, particularly in the preparation of tablets produced by pressing powders or granules. Flow regulators which can be employed include, but are not limited to, highly disperse silicon dioxide (Aerosil®) or dried starch.

Tablet and capsule dosage forms may further comprise a coating. Suitable coatings are film-forming polymers, such as, for example, those from the group of the cellulose derivatives (such as HPC (hydroxypropylcellulose), HPMC (hydroxypropoxymethylcellulose), MC (methylcellulose), HPMCAS (hydroxypropoxymethylcelluclose acetate succinate)), dextrins, starches, natural gums, such as, for example, gum arabic, xanthans, alginates, polyvinyl alcohol, polymethacrylates and derivatives thereof, such as, for example, Eudragit®, which may be applied to the tablet or capsule as solutions or suspensions by means of the various pharmaceutical conventional methods, such as, for example, film coating. The coating is typically applied as a solution/suspension which, in addition to any film-forming polymer present, may further comprise one or more adjuvants, such as hydrophilisers, plasticisers, surfactants, dyes and white pigments, such as, for example, titanium dioxide.

One skilled in the art will readily recognize that the appropriate pharmaceutically acceptable excipients are selected such that they are compatible with other excipients and do not bind with the active pharmaceutical ingredient or cause degradation.

The pharmaceutical formulation of the invention preferably is provided as a solid or semi-solid formulation. Formulations containing a second component that is solid or semi-solid at ambient temperature (e.g. a second component with a drop point of at least about 30° C.) are generally expected to have improved stability relative to liquid formulations. The reduced mobility of molecules in the solid phase reduces reactivity rates and therefore slows any degradation, compared to molecules in the liquid phase.

The pharmaceutical formulation can be obtained by
a) forming a melt comprising the first component and the second component described herein, wherein the forming a melt step comprises heating the second component; and
b) cooling the melt.

It will be appreciated that any of the above discussion relating to components of the pharmaceutical formulation may apply to any of the other aspects and embodiments of the invention. For example, any embodiment of the first component (the API), the second component and/or any other component of a pharmaceutical formulation as disclosed herein (e.g. antioxidant, crystallisation rate inhibitor) may be present in combination in a pharmaceutical formulation of the invention.

### Active Pharmaceutical Ingredient

The active pharmaceutical ingredient (API) is a MALT1 inhibitor. In particular, the active pharmaceutical ingredient is a compound of Formula (I)

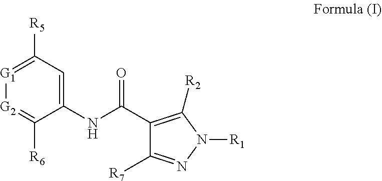

wherein
   R₁ is selected from the group consisting of
i) naphthalen-1-yl, optionally substituted with a fluoro or amino substituent;
   and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
   R₂ is selected from the group consisting of C₁₋₄alkyl, 1-methoxy-ethyl, difluoromethyl, fluoro, chloro, bromo, cyano, and trifluoromethyl;
   G₁ is N or C(R₄);
   G₂ is N or C(R₃); such that only one of G₁ and G₂ are N in any instance;
   R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
   R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy;
   and
xviii) morpholin-4-ylcarbonyl;
   R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
   or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
   R₆ is hydrogen, C₁₋₄alkyl, fluoro, 2-methoxy-ethoxy, chloro, cyano, or trifluoromethyl; and
   R₇ is hydrogen or fluoro;
or an enantiomer, diastereomer, solvate, or pharmaceutically acceptable salt form thereof.

Embodiments of the present invention include a pharmaceutical formulation as described herein, wherein the active pharmaceutical ingredient is a compound of Formula (I)

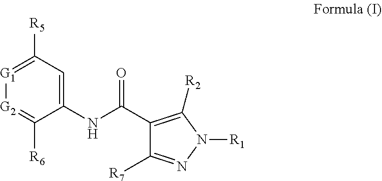

wherein
AA) R₁ is
   i) naphthalen-1-yl, optionally substituted with a fluoro or amino substituent;
      or
   ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, methoxymethyl, difluoromethyl, 1,1-difluoroethyl, hydroxymethyl, 1-hydroxyethyl, hydroxy, methoxy, fluoro, chloro, bromo, cyano, amino, methylamino, 4-oxotetrahydrofuran-2-yl, 5-oxopyrrolidin-2-yl, 1,4-dioxanyl, aminocarbonyl, methylaminocarbonyl, oxo, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
BB) R₁ is
   i) naphthalen-1-yl, optionally substituted with a fluoro or amino substituent;
      or
   ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, difluoromethyl, hydroxymethyl, 1-hydroxyethyl, hydroxy, fluoro, cyano, amino, aminocarbonyl, methylaminocarbonyl, oxo, tetrahydrofuran-2-yl, 3-hydroxy-pyrrolidin-1-yl, pyrrolidin-2-yl, 3-hydroxyazetidinyl, azetidin-3-yl, or azetidin-2-yl;
CC) R₁ is
   i) naphthalen-1-yl, optionally substituted with an amino or fluoro substituent;
      or
   ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from hydroxymethyl, 1-hydroxyethyl, hydroxy, fluoro, cyano, amino, or oxo;
DD) R₁ is
   i) naphthalen-1-yl, 4-amino-naphthalen-1-yl, 4-fluoronaphthalen-1-yl, or 5-fluoronaphthalen-1-yl;
      or
   ii) a heteroaryl selected from the group consisting of isoquinolin-1-yl, isoquinolin-4-yl, isoquinolin-5-yl, isoquinolin-8-yl, quinolin-7-yl, cinnolin-4-yl, imidazo[1,2-a]pyrazin-8-yl, phthalazin-1-yl, naphthyridin-5-yl, thieno[3,2-c]pyridin-4-yl, furo[3,2-c]pyridin-4-yl, furo[2,3-c]pyridin-7-yl, quinoxalin-5-yl, 1H-indazolylfuro[3,2-b]pyridin-7-yl, pyrazolo[1,5-a]pyrazin-4-yl, quinolin-4-yl, quinolin-5-yl, 1-aminoisoquinolin-4-yl, 1-oxo-1,2-dihydroisoquinolin-5-yl, benzo[d]thiazol-7-yl, 1-hydroxyisoquinolin-5-yl, benzo[d][1,2,3]thiadiazol-7-yl, thieno[2,3-c]pyridin-4-yl, pyrazolo[1,5-a]pyridin-4-yl, thieno[3,2-b]pyridin-7-yl, 2-oxo-1,2-dihydroquinolin-4-yl, 1-amino-8-fluoroisoquinolin-4-yl, 8-fluoroisoquinolin-4-yl, 1-cyanoisoquinolin-5-yl, pyrrolo[2,1-f][1,2,4]triazin-4-yl, 7-(1-hydroxyethyl)thieno[2,3-c]pyridin-4-yl, thieno[2,3-d]pyrimidin-4-yl, thieno[2,3-c]pyridin-7-yl, 1,7-naphthyridin-5-yl, pyrrolo[1,2-a]pyrazin-1-yl, imidazo[1,2-a]pyridin-5-yl, 1-aminocarbonyl-isoquinolin-4-yl, benzo[d]thiazol-4-yl, 8-fluoro-1-hydroxyisoquinolin-4-yl, thieno[3,2-d]pyrimidin-4-yl, 8-fluoroimidazo[1,2-a]pyridin-5-yl, 3-methylimidazo[1,2-a]pyridin-5-yl, 1-oxo-quinolin-4-yl, 8-aminoquinolin-5-yl, benzo[d]oxazol-4-yl, 3-methylthieno[3,2-b]pyridin-7-yl, 1-(hydroxymethyl)isoquinolin-4-yl, (3R-hydroxypyrrolidin-1-yl)isoquinolin-4-yl, (1-hydroxyethyl)isoquinolin-4-yl, 2-(difluoromethyl)quinolin-4-yl, 8-fluoroquinolin-5-yl, 1-hydroxyisoquinolin-4-yl, 1-(tetrahydrofuran-2-yl)isoquinolin-4-yl, 7-(difluoromethyl)thieno[2,3-c]pyridin-4-yl, 1-cyanoisoquinolin-4-yl, 1-(1(R)-hydroxyethyl)isoquinolin-4-yl, quinazolin-4-yl, 2-methylimidazo[1,2-a]pyridin-3-yl, thiazolo[5,4-d]pyrimidin-7-yl, 6-N-oxido-thieno[2,3-c]pyridin-4-yl, imidazo[1,2-a]pyridin-3-yl, furo[2,3-d]pyrimidin-4-yl, 2-fluoroquinolin-5-yl, benzo[d]isothiazol-3-yl, 7-methylpyrazolo[1,5-a]pyridin-4-yl, 1-(hydroxyethyl)quinolin-4-yl, 1-(methoxymethyl)isoquinolin-4-yl, 1-fluoroisoquinolin-4-yl, 1-(difluoromethyl)isoquinolin-4-yl, 8-fluoroquinolin-4-yl, 1-(tetrahydrofuran-2(R)-yl)isoquinolin-4-yl, 2-amino-[1,2,4]triazolo[1,5-a]pyridin-5-yl, 1-(4-oxotetrahydrofuran-2-yl)isoquinolin-4-yl, 2-(aminocarbonyl)quinolin-4-yl, 1H-indazol-7-yl, 1-(1,4-dioxan-2-yl)isoquinolin-4-yl, 2-methylimidazo[1,2-a]pyridin-5-yl, 1-chloroisoquinolin-4-yl, 2-cyanoquinolin-4-yl, 8-fluoro-1-(methylamino)isoquinolin-4-yl, benzo[d]isoxazol-3-yl, 2-aminobenzo[d]thiazol-7-yl, 1,7-naphthyridin-4-yl, imidazo[1,2-a]pyrazin-5-yl, (N-(methyl)formamido)methyl)isoquinolin-4-yl, [1,2,4]triazolo[1,5-a]pyridin-5-yl, 2-methylbenzo[d]oxazol-7-yl, 1,5-naphthyridin-4-yl, 5-oxopyrrolidin-2-ylisoquinolin-4-yl, 1-methyl-1H-indazol-3-yl, 1-(1,1-difluoroethyl)isoquinolin-4-yl, 1-(1(*S)-hydroxyethyl)isoquinolin-4-yl, 1-(methylamino)isoquinolin-4-yl, 4-fluoroisoquinolin-1-yl, 1H-pyrazolo[4,3-b]pyridin-7-yl, 5-fluoroquinolin-8-yl, 6-fluoroimidazo[1,2-a]pyridin-5-yl, 2-methylfuro[3,2-b]pyridin-7-yl, 8-(difluoromethyl)quinolin-5-yl, 1-(4-oxotetrahydrofuran-2R-yl)isoquinolin-4-yl, 1-(dimethylamino)isoquinolin-4-yl, 1-methyl-1H-pyrazolo[3,4-c]pyridin-7-yl, 2-methyl-[1,2,4]triazolo[1,5-a]pyridin-5-yl, 2-methoxyquinolin-4-yl, imidazo[1,2-a]pyrimidin-5-yl, 2-(difluoromethyl)thieno[2,3-c]pyridin-4-yl, 1-(1-ethoxyethyl)isoquinolin-4-yl, 2-(azetidin-2-yl)quinolin-4-yl, 2-methylbenzo[d]thiazol-7-yl, 2-acetylquinolin-4-yl, 1-(methylthio)isoquinolin-4-yl, 2-aminoquinolin-5-yl, 1-methoxyisoquinolin-5-yl, imidazo[1,2-b]pyridazin-6-yl, 1-(pyrrolidin-2-yl)isoquinolin-4-yl, 4-(difluoromethyl)quinolin-5-yl, 1-acetylisoquinolin-5-yl, 1-(azetidin-2-yl)isoquinolin-4-yl, 1-ethoxyisoquinolin-4-yl, 1-methyl-1H-pyrazolo[3,4-b]pyridin-4-yl, 1-aminoisoquinolin-5-yl, 1-methyl-1H-indazol-4-yl, 2-aminoquinolin-4-yl, 2-oxo-1,2-dihydroquinolin-5-yl, 1-(azetidin-3-yl)isoquinolin-4-yl, 2-methylthieno[3,2-b]pyridin-7-yl, benzo[d][1,2,3]thiadiazol-4-yl, 1-(1(S)-hydroxyethyl)isoquinolin-5-yl, imidazo[1,2-a]pyridin-8-yl, 2-methyl-1-oxo-1,2-dihydroisoquinolin-5-yl, 2-(tetrahydrofuran-2-yl)quinolin-5-yl, 1-(1(R)-hydroxyethyl)isoquinolin-5-yl, 1,6-naphthyridin-4-yl, 1H-pyrazolo[3,4-d]pyrimidin-4-yl, 2-aminocarbonyl-quinolin-5-yl, 2-chloroquinolin-5-yl, 2-chloroquinolin-4-yl, 2-cyanoquinolin-5-yl, 2-methoxyquinolin-5-yl, 2-methylbenzo[d]oxazol-4-yl, 2-(difluoromethyl)quinolin-5-yl, 2-(azetidin-2-yl)quinolin-5-yl, 1-(azetidin-2-yl)isoquinolin-5-yl, 1,5-bis(tetrahydrofuran-2-yl)isoquinolin-4-yl, 1-oxo-1,2-dihydroisoquinolin-4-yl, 2-methyl-1-oxo-1,2-dihydroisoquinolin-4-yl, 1-(3-hydroxyazetidin-1-yl)isoquinolin-4-yl, 8-fluoro-1-(3-hydroxyazetidin-1-yl)isoquinolin-4-yl, (R)-8-fluoro-1-(3-hydroxypyrrolidin-1-yl)isoquinolin-4-yl, (S)-8-fluoro-1-(3-hydroxypyrrolidin-1-yl)isoquinolin-4-yl, 3-hydroxyazetidin-1-yl)thieno[2,3-c]pyridin-4-yl, 8-(3-hydroxyazetidin-1-yl)imidazo[1,2-a]pyridin-5-yl, 7-(3-hydroxyazetidin-1-yl)pyrazolo[1,5-a]pyridin-4-yl, 1-(3-hydroxyazetidin-1-yl)isoquinolin-5-yl, and 1-(1-t-butoxycarbonylazetidin-2-yl)isoquinolin-5-yl;
EE) R₁ is
   i) naphthalen-1-yl or 4-fluoronaphthalen-1-yl, 4-amino-naphthalen-1-yl or 5-fluoronaphthalen-1-yl;
      or
   ii) a heteroaryl selected from the group consisting of thieno[3,2-c]pyridin-4-yl, isoquinolin-4-yl, 8-fluoroquinolin-4-yl, furo[3,2-c]pyridin-4-yl, quinolin-5-yl, furo[2,3-c]pyridin-7-yl, benzofuran-4-yl 1,7-naphthyridin-5-yl, pyrrolo[1,2-a]pyrazin-1-yl, imidazo[1,2-a]pyridin-5-yl, 1-aminocarbonyl-isoquinolin-4-yl, pyrrolo[1,2-a]pyrazin-1-yl, benzo[d]thiazol-4-yl, 8-fluoro-1-hydroxyisoquinolin-4-yl, thieno[3,2-d]pyrimidin-4-yl, 8-fluoroimidazo[1,2-a]pyridin-5-yl, 3-methylimidazo[1,2-a]pyridin-5-yl, 1-aminoisoquinolin-4-yl, 1-oxo-quinolin-4-yl, 8-aminoquinolin-5-yl, benzo[d]oxazol-4-yl, 3-methylthieno[3,2-b]pyridin-7-yl, 1-(hydroxymethyl)isoquinolin-4-yl, (3R-hydroxypyrrolidin-1-yl)isoquinolin-4-yl, (1-hydroxyethyl)isoquinolin-4-yl, 8-fluoroisoquinolin-4-yl, 2-(difluoromethyl)quinolin-4-yl, 8-fluoroquinolin-5-yl, 1-hydroxyisoquinolin-4-yl, benzo[d]thiazol-4-yl, 1-aminoisoquinolin-4-yl, 1-(tetrahydrofuran-2-yl)isoquinolin-4-yl, 7-(difluoromethyl)thieno[2,3-c]pyridin-4-yl, 1-(1-hydroxyethyl)isoquinolin-4-yl, 1-cyanoisoquinolin-4-yl, 1-(1(R)-hydroxyethyl)isoquinolin-4-yl, quinazolin-4-yl, 2-methylimidazo[1,2-a]pyridin-3-yl, thiazolo[5,4-d]pyrimidin-7-yl, imidazo[1,2-a]pyridin-5-yl, benzo[d][1,2,3]thiadiazol-7-yl, 6-N-oxido-thieno[2,3-c]pyridin-4-yl, imidazo[1,2-a]pyridin-3-yl, furo[2,3-d]pyrimidin-4-yl, 2-fluoroquinolin-5-yl, isoquinolin-5-yl, benzo[d]isothiazol-3-yl, 7-methylpyrazolo[1,5-a]pyridin-4-yl, 1-oxo-1,2-dihydroisoquinolin-4-yl, 2-methyl-1-oxo-1,2-dihydroisoquinolin-4-yl, 1-(3-hydroxyazetidin-1-yl)isoquinolin-4-yl, 8-fluoro-1-(3-hydroxyazetidin-1-yl)isoquinolin-4-yl, (R)-8-fluoro-1-(3-hydroxypyrrolidin-1-yl)isoquinolin-4-yl, (S)-8-fluoro-1-(3-hydroxypyrrolidin-1-yl)isoquinolin-4-yl, 3-hydroxyazetidin-1-yl)thieno[2,3-c]pyridin-4-yl, 8-(3-hydroxyazetidin-1-yl)imidazo[1,2-a]pyridin-5-yl, 7-(3-hydroxyazetidin-1-yl)pyrazolo[1,5-a]pyridin-4-yl, 1-(3-hydroxyazetidin-1-yl)isoquinolin-5-yl, and 1-(hydroxyethyl)quinolin-4-yl;
FF) R₂ is independently selected from the group consisting of methyl, isopropyl, cyano, bromo, chloro, and trifluoromethyl;
GG) R₂ is independently selected from the group consisting of methyl, isopropyl, cyano, and trifluoromethyl;
HH) R₂ is trifluoromethyl;
II) R₃ is independently selected from the group consisting of trifluoromethyl, cyano, methylcarbonyl, methylthio, methylsulfinyl, methanesulfonyl, and chloro; or, when G₁ is N, R₃ is further selected from C₁₋₄alkoxycarbonyl;
JJ) R₃ is independently selected from the group consisting of trifluoromethyl, cyano, and chloro;
KK) G₂ is N or C(R₃), wherein R₃ is chloro;
LL) G₂ is N;
MM) R₄ is selected from the group consisting of
   i) hydrogen, when G₂ is N;
   ii) C₁₋₄alkoxy;
   iii) cyano;
   iv) cyclopropyloxy;
   v) carboxy;
   vi) a heteroaryl selected from the group consisting of triazolyl, oxazolyl, pyrazolyl, thiazolyl, oxadiazolyl, imidazolyl, and pyrimidin-4-yl, wherein the heteroaryl is optionally substituted with one or two substituents independently selected from the group consisting of C₁₋₄alkyl, carboxy, methoxycarbonyl, hydroxymethyl, aminocarbonyl, (dimethylamino)methyl, amino, methoxymethyl, trifluoromethyl, amino(C₂₋₄alkyl)amino, and cyano;
   vii) 1-methyl-piperidin-4-yloxy;
   viii) 4-methyl-piperazin-1-ylcarbonyl;
   ix) (4-aminobutyl)aminocarbonyl;
   x) (4-amino)butoxy;
   xi) methoxycarbonyl;
   xii) 5-chloro-6-(methoxycarbonyl)pyridin-3-ylaminocarbonyl;
   xiii) 1,1-dioxo-isothiazolidin-2-yl;
      and
   xiv) morpholin-4-ylcarbonyl;
NN) R₄ is selected from the group consisting of
   i) hydrogen;
   ii) C₁₋₄alkoxy;
   iii) cyano;
   iv) cyclopropyloxy;
   v) a heteroaryl selected from the group consisting of triazolyl, oxazolyl, pyrazolyl, thiazolyl, oxadiazolyl, and imidazolyl, wherein the heteroaryl is optionally substituted with one or two substituents independently selected from the group consisting of methyl, carboxy, methoxycarbonyl, hydroxymethyl, aminocarbonyl, (dimethylamino)methyl, and amino, methoxymethyl;
   vi) (4-amino)butoxy;
   vii) methoxycarbonyl;
   viii) 5-chloro-6-(methoxycarbonyl)pyridin-3-ylaminocarbonyl;
      and
   ix) 1,1-dioxo-isothiazolidin-2-yl;
OO) R₄ is selected from the group consisting of
   i) methoxy;
   ii) a heteroaryl independently selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 4-amino-2H-1,2,3-triazol-2-yl, 4-(hydroxymethyl)-1H-pyrazol-1-yl, 4-(hydroxymethyl)-2H-1,2,3-triazol-2-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, 1,3,4-oxadiazol-2-yl, 2-methyl-2H-tetrazol-5-yl, 5-amino-1-methyl-1H-pyrazol-3-yl, 4-(hydroxymethyl)-1H-pyrazol-1-yl, 4-cyano-2H-1,2,3-triazol-2-yl, 5-amino-1H-1,2,3-triazol-1-yl, 2H-1,2,3-triazol-4-yl, 2H-tetrazol-5-yl, 4-(aminomethyl)-1H-pyrazol-1-yl, 4-(methoxymethyl)-2H-1,2,3-triazol-2-yl, 2-methyl-2H-tetrazol-5-yl, and 4-methyl-1H-1,2,3-triazol-1-yl;
      and
   iii) methoxycarbonyl;
PP) R₄ is independently selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 1H-imidazol-2-yl, 4-amino-2H-1,2,3-triazol-2-yl, 4-(hydroxymethyl)-1H-pyrazol-1-yl, 4-(hydroxymethyl)-2H-1,2,3-triazol-2-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, and 1,3,4-oxadiazol-2-yl;
QQ) R₅ is hydrogen, chloro, fluoro, bromo, cyano, methyl, ethyl, or trifluoromethyl; or, R₄ and R₅ may be taken together to form 8-chloro-4-methyl-3-oxo-3,4-dihydro-2H-benzo[b][1,4]oxazin-6-yl or 8-chloro-3-oxo-3,4-dihydro-2H-benzo[b][1,4]oxazin-6-yl;
RR) R₅ is hydrogen, chloro, bromo, cyano, or trifluoromethyl; or, R₄ and R₅ may be taken together to form 8-chloro-4-methyl-3-oxo-3,4-dihydro-2H-benzo[b][1,4]oxazin-6-yl or 8-chloro-3-oxo-3,4-dihydro-2H-benzo[b][1,4]oxazin-6-yl;
SS) R₅ is hydrogen, chloro, bromo, or cyano;
TT) R₅ is hydrogen, chloro, or cyano;
UU) R₆ is hydrogen or methyl;
VV) R₇ is hydrogen;
   and any combination of embodiments AA) though VV) above, provided it is understood that combinations in which different embodiments of the same substituent would be combined are excluded; such that only one of G₁ and G₂ are N in any instance; or an enantiomer, diastereomer, solvate, or pharmaceutically acceptable salt form thereof.

Embodiments of the present invention include a pharmaceutical formulation as described herein wherein the active pharmaceutical ingredient is a compound of Formula (I)

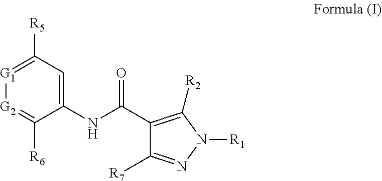

wherein
   R₁ is selected from the group consisting of
   i) naphthalen-1-yl, 4-amino-naphthalen-1-yl, or 4-fluoronaphthalen-1-yl, 5-fluoronaphthalen-1-yl;
      and
   ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, methoxymethyl, difluoromethyl, 1,1-difluoroethyl, hydroxymethyl, 1-hydroxyethyl, hydroxy, methoxy, fluoro, chloro, bromo, cyano, amino, methylamino, 4-oxotetrahydrofuran-2-yl, 5-oxopyrrolidin-2-yl, 1,4-dioxanyl, aminocarbonyl, methylaminocarbonyl, oxo, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
   R₂ is independently selected from the group consisting of methyl, isopropyl, cyano, bromo, chloro, and trifluoromethyl;
   G₁ is N or C(R₄);
   G₂ is N or C(R₃); such that only one of G₁ and G₂ is N in any instance;
   R₃ is independently selected from the group consisting of trifluoromethyl, cyano, methylcarbonyl, methylthio, methylsulfinyl, methanesulfonyl, and chloro; or, when G₁ is N, R₃ is further selected from C₁₋₄alkoxycarbonyl;
   R₄ is independently selected from the group consisting of
i) hydrogen, when G₂ is N;
ii) C₁₋₄alkoxy;
iii) cyano;
iv) cyclopropyloxy;
v) carboxy;
vi) a heteroaryl selected from the group consisting of triazolyl, oxazolyl, pyrazolyl, thiazolyl, oxadiazolyl, imidazolyl, and 2-amino-pyrimidin-4-yl, wherein the heteroaryl is optionally substituted with one or two substituents independently selected from the group consisting of C₁₋₄alkyl, carboxy, methoxycarbonyl, hydroxymethyl, aminocarbonyl, (dimethylamino)methyl, amino, methoxymethyl, trifluoromethyl, amino(C₂₋₄alkyl)amino, and cyano;
vii) 1-methyl-piperidin-4-yloxy;
viii) 4-methyl-piperazin-1-ylcarbonyl;
ix) (4-aminobutyl)aminocarbonyl;
x) (4-amino)butoxy;
xi) methoxycarbonyl;
xii) 5-chloro-6-(methoxycarbonyl)pyridin-3-ylaminocarbonyl;
xiii) 1,1-dioxo-isothiazolidin-2-yl;
   and
xiv) morpholin-4-ylcarbonyl;
   R₅ is hydrogen, chloro, fluoro, bromo, cyano, methyl, ethyl, or trifluoromethyl; or, R₄ and R₅ may be taken together to form 8-chloro-4-methyl-3-oxo-3,4-dihydro-2H-benzo[b][1,4]oxazin-6-yl or 8-chloro-3-oxo-3,4-dihydro-2H-benzo[b][1,4]oxazin-6-yl;
   R₆ is hydrogen or methyl; and
   R₇ is hydrogen;
or an enantiomer, diastereomer, solvate, or pharmaceutically acceptable salt form thereof.

Embodiments of the present invention include a pharmaceutical formulation as described herein, wherein the active pharmaceutical ingredient is a compound of Formula (I)

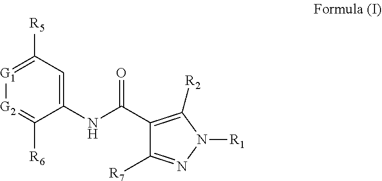

wherein
   R₁ is selected from the group consisting of
i) naphthalen-1-yl, optionally substituted with a fluoro or amino substituent;
   or
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, difluoromethyl, hydroxymethyl, 1-hydroxyethyl, hydroxy, fluoro, cyano, amino, aminocarbonyl, methylaminocarbonyl, oxo, tetrahydrofuran-2-yl, 3-hydroxy-pyrrolidin-1-yl, pyrrolidin-2-yl, 3-hydroxyazetidinyl, azetidin-3-yl, or azetidin-2-yl;
   R₂ is selected from the group consisting of methyl, isopropyl, cyano, and trifluoromethyl;
   G₁ is N or C(R₄);
   G₂ is N or C(R₃); such that only one of G₁ and G₂ is N in any instance;
   R₃ is independently selected from the group consisting of trifluoromethyl, cyano, and chloro;
   R₄ is independently selected from the group consisting of
   i) hydrogen;
   ii) C₁₋₄alkoxy;
   iii) cyano;
   iv) cyclopropyloxy;
   v) a heteroaryl selected from the group consisting of triazolyl, oxazolyl, pyrazolyl, thiazolyl, oxadiazolyl, and imidazolyl, wherein the heteroaryl is optionally substituted with one or two substituents independently selected from the group consisting of methyl, carboxy, methoxycarbonyl, hydroxymethyl, aminocarbonyl, (dimethylamino)methyl, and amino, methoxymethyl;
   vi) (4-amino)butoxy;
   vii) methoxycarbonyl;
   viii) 5-chloro-6-(methoxycarbonyl)pyridin-3-ylaminocarbonyl;
      and
   ix) 1,1-dioxo-isothiazolidin-2-yl;
   R₅ is hydrogen, chloro, bromo, or cyano;
   R₆ is hydrogen or methyl;
   R₇ is hydrogen;
   or an enantiomer, diastereomer, solvate, or pharmaceutically acceptable salt form thereof.

In some embodiments, a compound of Formula (I) is other than:
a compound wherein R₁ is isoquinolin-8-yl, R₂ is trifluoromethyl, G₁ is C(R₄) wherein R₄ is 2H-1,2,3-triazol-2-yl, G₂ is N, and R₅ is hydrogen;
a compound wherein R₁ is isoquinolin-8-yl, R₂ is trifluoromethyl, G₁ is C(R₄) wherein R₄ is 1H-imidazol-1-yl, G₂ is N, and R₅ is chloro;
a compound wherein R₁ is isoquinolin-8-yl, R₂ is trifluoromethyl, G₁ is C(R₄) wherein R₄ is 1H-1,2,3-triazol-1-yl, G₂ is N, and R₅ is hydrogen; and
a compound wherein R₁ is isoquinolin-8-yl, R₂ is trifluoromethyl, G₁ is C(R₄) wherein R₄ is hydrogen, G₂ is N, and R₅ is fluoro.

Embodiments of the present invention include a pharmaceutical formulation as described herein wherein the active pharmaceutical ingredient is a compound of Formula (I)

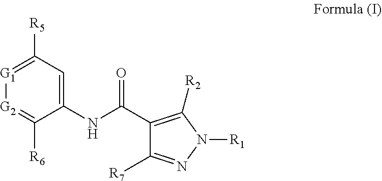

wherein
   R₁ is selected from the group consisting of
i) naphthalen-1-yl, optionally substituted with a fluoro or amino substituent;
   and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from hydroxymethyl, 1-hydroxyethyl, hydroxy, fluoro, cyano, amino, oxo, 3-hydroxy-pyrrolidin-1-yl, pyrrolidin-2-yl, 3-hydroxyazetidinyl, azetidin-3-yl, or azetidin-2-yl;
   R₂ is selected from the group consisting of methyl, isopropyl, cyano, and trifluoromethyl;
   G₁ is N or C(R₄);
   G₂ is N or C(R₃); such that only one of G₁ and G₂ is N in any instance;
   R₃ is independently selected from the group consisting of trifluoromethyl, cyano, and chloro;
   R⁴ is selected from the group consisting of
i) methoxy;
ii) a heteroaryl selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 4-amino-2H-1,2,3-triazol-2-yl, 4-(hydroxymethyl)-1H-pyrazol-1-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, 1,3,4-oxadiazol-2-yl, 2-methyl-2H-tetrazol-5-yl, 5-amino-1-methyl-1H-pyrazol-3-yl, 4-(hydroxymethyl)-1H-pyrazol-1-yl, 4-cyano-2H-1,2,3-triazol-2-yl, 5-amino-1H-1,2,3-triazol-1-yl, 2H-1,2,3-triazol-4-yl, 2H-tetrazol-5-yl, 4-(aminomethyl)-1H-pyrazol-1-yl, 4-(methoxymethyl)-2H-1,2,3-triazol-2-yl, 2-methyl-2H-tetrazol-5-yl, and 4-methyl-1H-1,2,3-triazol-1-yl;
   and
iii) methoxycarbonyl;
   R₅ is hydrogen, chloro, or cyano;
   R₆ is hydrogen or methyl;
   R₇ is hydrogen;
or an enantiomer, diastereomer, solvate, or pharmaceutically acceptable salt form thereof.

Embodiments of the present invention include a pharmaceutical formulation as described herein wherein the active pharmaceutical ingredient is a compound of Formula (I)

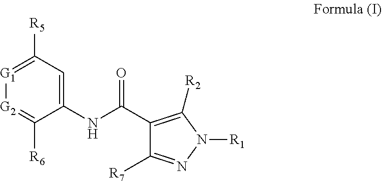

wherein
   R₁ is independently selected from the group consisting of
i) naphthalen-1-yl, 4-amino-naphthalen-1-yl, 4-fluoronaphthalen-1-yl, or 5-fluoronaphthalen-1-yl;
   and
ii) a heteroaryl selected from the group consisting of isoquinolin-1-yl, isoquinolin-4-yl, isoquinolin-5-yl, isoquinolin-8-yl, quinolin-7-yl, cinnolin-4-yl, imidazo[1,2-a]pyrazin-8-yl, phthalazin-1-yl, naphthyridin-5-yl, thieno[3,2-c]pyridin-4-yl, furo[3,2-c]pyridin-4-yl, furo[2,3-c]pyridin-7-yl, quinoxalin-5-yl, 1H-indazolylfuro[3,2-b]pyridin-7-yl, pyrazolo[1,5-a]pyrazin-4-yl, quinolin-4-yl, quinolin-5-yl, 1-aminoisoquinolin-4-yl, 1-oxo-1,2-dihydroisoquinolin-5-yl, benzo[d]thiazol-7-yl, 1-hydroxyisoquinolin-5-yl, benzo[d][1,2,3]thiadiazol-7-yl, thieno[2,3-c]pyridin-4-yl, pyrazolo[1,5-a]pyridin-4-yl, thieno[3,2-b]pyridin-7-yl, 2-oxo-1,2-dihydroquinolin-4-yl, 1-amino-8-fluoroisoquinolin-4-yl, 8-fluoroisoquinolin-4-yl, 1-cyanoisoquinolin-5-yl, pyrrolo[2,1-f][1,2,4]triazin-4-yl, 7-(1-hydroxyethyl)thieno[2,3-c]pyridin-4-yl, thieno[2,3-d]pyrimidin-4-yl, thieno[2,3-c]pyridin-7-yl, 1,7-naphthyridin-5-yl, pyrrolo[1,2-a]pyrazin-1-yl, imidazo[1,2-a]pyridin-5-yl, 1-aminocarbonyl-isoquinolin-4-yl, benzo[d]thiazol-4-yl, 8-fluoro-1-hydroxyisoquinolin-4-yl, thieno[3,2-d]pyrimidin-4-yl, 8-fluoroimidazo[1,2-a]pyridin-5-yl, 3-methylimidazo[1,2-a]pyridin-5-yl, 1-oxo-quinolin-4-yl, 8-aminoquinolin-5-yl, benzo[d]oxazol-4-yl, 3-methylthieno[3,2-b]pyridin-7-yl, 1-(hydroxymethyl)isoquinolin-4-yl, (3R-hydroxypyrrolidin-1-yl)isoquinolin-4-yl, (1-hydroxyethyl)isoquinolin-4-yl, 2-(difluoromethyl)quinolin-4-yl, 8-fluoroquinolin-5-yl, 1-hydroxyisoquinolin-4-yl, 1-(tetrahydrofuran-2-yl)isoquinolin-4-yl, 7-(difluoromethyl)thieno[2,3-c]pyridin-4-yl, 1-cyanoisoquinolin-4-yl, 1-(1(R)-hydroxyethyl)isoquinolin-4-yl, quinazolin-4-yl, 2-methylimidazo[1,2-a]pyridin-3-yl, thiazolo[5,4-d]pyrimidin-7-yl, 6-N-oxido-thieno[2,3-c]pyridin-4-yl, imidazo[1,2-a]pyridin-3-yl, furo[2,3-d]pyrimidin-4-yl, 2-fluoroquinolin-5-yl, benzo[d]isothiazol-3-yl, 7-methylpyrazolo[1,5-a]pyridin-4-yl, 1-(hydroxyethyl)quinolin-4-yl, 1-(methoxymethyl)isoquinolin-4-yl, 1-fluoroisoquinolin-4-yl, 1-(difluoromethyl)isoquinolin-4-yl, 8-fluoroquinolin-4-yl, 8-fluoroquinolin-5-yl, 1-(tetrahydrofuran-2(R)-yl)isoquinolin-4-yl, 2-amino-[1,2,4]triazolo[1,5-a]pyridin-5-yl, 1-(4-oxotetrahydrofuran-2-yl)isoquinolin-4-yl, 2-(aminocarbonyl)quinolin-4-yl, 1H-indazol-7-yl, 1-(1,4-dioxan-2-yl)isoquinolin-4-yl, 2-methylimidazo[1,2-a]pyridin-5-yl, 1-chloroisoquinolin-4-yl, 2-cyanoquinolin-4-yl, 8-fluoro-1-(methylamino)isoquinolin-4-yl, benzo[d]isoxazol-3-yl, 2-aminobenzo[d]thiazol-7-yl, 1,7-naphthyridin-4-yl, imidazo[1,2-a]pyrazin-5-yl, (N-(methyl)formamido)methyl)isoquinolin-4-yl, [1,2,4]triazolo[1,5-a]pyridin-5-yl, 2-methylbenzo[d]oxazol-7-yl, 1,5-naphthyridin-4-yl, 5-oxopyrrolidin-2-ylisoquinolin-4-yl, 1-methyl-1H-indazol-3-yl, 1-(tetrahydrofuran-2-yl)isoquinolin-4-yl, 1-(1,1-difluoroethyl)isoquinolin-4-yl, 1-(1(^{x}S)-hydroxyethyl)isoquinolin-4-yl, 1-(methylamino)isoquinolin-4-yl, 4-fluoroisoquinolin-1-yl, 1H-pyrazolo[4,3-b]pyridin-7-yl, 5-fluoroquinolin-8-yl, 6-fluoroimidazo[1,2-a]pyridin-5-yl, 2-methylfuro[3,2-b]pyridin-7-yl, 8-(difluoromethyl)quinolin-5-yl, 1-(4-oxotetrahydrofuran-2R-yl)isoquinolin-4-yl, 1-(dimethylamino)isoquinolin-4-yl, 1-methyl-1H-pyrazolo[3,4-c]pyridin-7-yl, 2-methyl-[1,2,4]triazolo[1,5-a]pyridin-5-yl, 2-methoxyquinolin-4-yl, imidazo[1,2-a]pyrimidin-5-yl, 2-(difluoromethyl)thieno[2,3-c]pyridin-4-yl, 1-(1-ethoxyethyl)isoquinolin-4-yl, 2-(azetidin-2-yl)quinolin-4-yl, 2-methylbenzo[d]thiazol-7-yl, 2-acetylquinolin-4-yl, 1-(methylthio)isoquinolin-4-yl, 2-aminoquinolin-5-yl, 1-methoxyisoquinolin-5-yl, imidazo[1,2-b]pyridazin-6-yl, 1-(pyrrolidin-2-yl)isoquinolin-4-yl, 4-(difluoromethyl)quinolin-5-yl, 1-acetylisoquinolin-5-yl, 1-(azetidin-2-yl)isoquinolin-4-yl, 1-ethoxyisoquinolin-4-yl, 1-methyl-1H-pyrazolo[3,4-b]pyridin-4-yl, 1-aminoisoquinolin-5-yl, 1-methyl-1H-indazol-4-yl, 2-aminoquinolin-4-yl, 2-oxo-1,2-dihydroquinolin-5-yl, 1-(azetidin-3-yl)isoquinolin-4-yl, 2-methylthieno[3,2-b]pyridin-7-yl, benzo[d][1,2,3]thiadiazol-4-yl, 1-(1(S)-hydroxyethyl)isoquinolin-5-yl, imidazo[1,2-a]pyridin-8-yl, 2-methyl-1-oxo-1,2-dihydroisoquinolin-5-yl, 2-(tetrahydrofuran-2-yl)quinolin-5-yl, 1-(1(R)-hydroxyethyl)isoquinolin-5-yl, 1,6-naphthyridin-4-yl, 1H-pyrazolo[3,4-d]pyrimidin-4-yl, 2-aminocarbonyl-quinolin-5-yl, 2-chloroquinolin-5-yl, 2-chloroquinolin-4-yl, 2-cyanoquinolin-5-yl, 2-methoxyquinolin-5-yl, 2-methylbenzo[d]oxazol-4-yl, 2-(difluoromethyl)quinolin-5-yl, 2-(azetidin-2-yl)quinolin-5-yl, 1-(azetidin-2-yl)isoquinolin-5-yl, 1,5-bis(tetrahydrofuran-2-yl)isoquinolin-4-yl, 1-oxo-1,2-dihydroisoquinolin-4-yl, 2-methyl-1-oxo-1,2-dihydroisoquinolin-4-yl, 1-(3-hydroxyazetidin-1-yl)isoquinolin-4-yl, 8-fluoro-1-(3-hydroxyazetidin-1-yl)isoquinolin-4-yl, (R)-8-fluoro-1-(3-hydroxypyrrolidin-1-yl)isoquinolin-4-yl, (S)-8-fluoro-1-(3-hydroxypyrrolidin-1-yl)isoquinolin-4-yl, 3-hydroxyazetidin-1-yl)thieno[2,3-c]pyridin-4-yl, 8-(3-hydroxyazetidin-1-yl)imidazo[1,2-a]pyridin-5-yl, 7-(3-hydroxyazetidin-1-yl)pyrazolo[1,5-a]pyridin-4-yl, 1-(3-hydroxyazetidin-1-yl)isoquinolin-5-yl, and 1-(1-t-butoxycarbonylazetidin-2-yl)isoquinolin-5-yl;
   R₂ is trifluoromethyl;
   G₁ is N or C(R₄);
   G₂ is N or C(R₃); such that only one of G₁ and G₂ is N in any instance;
   R₃ is independently selected from the group consisting of trifluoromethyl, cyano, and chloro;
   R₄ is independently selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 1H-imidazol-2-yl, 4-amino-2H-1,2,3-triazol-2-yl, 4-(hydroxymethyl)-1H-pyrazol-1-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, and 1,3,4-oxadiazol-2-yl;
   R₅ is hydrogen, chloro, bromo, or cyano;
   R₆ is hydrogen or methyl;
   R₇ is hydrogen;
or an enantiomer, diastereomer, solvate, or pharmaceutically acceptable salt form thereof.

Embodiments of the present invention include pharmaceutical formulations as described herein wherein the active pharmaceutical ingredient is a compound of Formula (I)

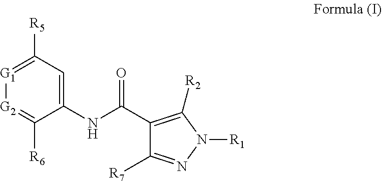

wherein
   R₁ is independently selected from the group consisting of
i) naphthalen-1-yl, 4-amino-naphthalen-1-yl, 4-fluoronaphthalen-1-yl, or 5-fluoronaphthalen-1-yl;
   and
ii) a heteroaryl selected from the group consisting of thieno[3,2-c]pyridin-4-yl, isoquinolin-4-yl, 8-fluoroquinolin-4-yl, furo[3,2-c]pyridin-4-yl, quinolin-5-yl, furo[2,3-c]pyridin-7-yl, benzofuran-4-yl 1,7-naphthyridin-5-yl, pyrrolo[1,2-a]pyrazin-1-yl, imidazo[1,2-a]pyridin-5-yl, 1-aminocarbonyl-isoquinolin-4-yl, pyrrolo[1,2-a]pyrazin-1-yl, benzo[d]thiazol-4-yl, 8-fluoro-1-hydroxyisoquinolin-4-yl, thieno[3,2-d]pyrimidin-4-yl, 8-fluoroimidazo[1,2-a]pyridin-5-yl, 3-methylimidazo[1,2-a]pyridin-5-yl, 1-aminoisoquinolin-4-yl, 1-oxo-quinolin-4-yl, 8-aminoquinolin-5-yl, benzo[d]oxazol-4-yl, 3-methylthieno[3,2-b]pyridin-7-yl, 1-(hydroxymethyl)isoquinolin-4-yl, (3R-hydroxypyrrolidin-1-yl)isoquinolin-4-yl, (1-hydroxyethyl)isoquinolin-4-yl, 8-fluoroisoquinolin-4-yl, 2-(difluoromethyl)quinolin-4-yl, 8-fluoroquinolin-5-yl, 1-hydroxyisoquinolin-4-yl, benzo[d]thiazol-4-yl, 1-aminoisoquinolin-4-yl, 1-(tetrahydrofuran-2-yl)isoquinolin-4-yl, 7-(difluoromethyl)thieno[2,3-c]pyridin-4-yl, 1-(1-hydroxyethyl)isoquinolin-4-yl, 1-cyanoisoquinolin-4-yl, 1-(1(R)-hydroxyethyl)isoquinolin-4-yl, quinazolin-4-yl, 2-methylimidazo[1,2-a]pyridin-3-yl, thiazolo[5,4-d]pyrimidin-7-yl, imidazo[1,2-a]pyridin-5-yl, benzo[d][1,2,3]thiadiazol-7-yl, 6-N-oxido-thieno[2,3-c]pyridin-4-yl, imidazo[1,2-a]pyridin-3-yl, furo[2,3-d]pyrimidin-4-yl, 2-fluoroquinolin-5-yl, isoquinolin-5-yl, benzo[d]isothiazol-3-yl, 7-methylpyrazolo[1,5-a]pyridin-4-yl, 1-oxo-1,2-dihydroisoquinolin-4-yl, 2-methyl-1-oxo-1,2-dihydroisoquinolin-4-yl, 1-(3-hydroxyazetidin-1-yl)isoquinolin-4-yl, 8-fluoro-1-(3-hydroxyazetidin-1-yl)isoquinolin-4-yl, (R)-8-fluoro-1-(3-hydroxypyrrolidin-1-yl)isoquinolin-4-yl, (S)-8-fluoro-1-(3-hydroxypyrrolidin-1-yl)isoquinolin-4-yl, 3-hydroxyazetidin-1-yl)thieno[2,3-c]pyridin-4-yl, 8-(3-hydroxyazetidin-1-yl)imidazo[1,2-a]pyridin-5-yl, 7-(3-hydroxyazetidin-1-yl)pyrazolo[1,5-a]pyridin-4-yl, 1-(3-hydroxyazetidin-1-yl)isoquinolin-5-yl and 1-(hydroxyethyl)quinolin-4-yl;
   R₂ is trifluoromethyl;
   G₁ is N or C(R₄);
   G₂ is N or C(R₃); such that only one of G₁ and G₂ is N in any instance;
   R₃ is independently selected from the group consisting of trifluoromethyl, cyano, and chloro;
   R₄ is independently selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 1H-imidazol-2-yl, 4-amino-2H-1,2,3-triazol-2-yl, 4-(hydroxymethyl)-1H-pyrazol-1-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, and 1,3,4-oxadiazol-2-yl;
   R₅ is hydrogen, chloro, or cyano;
   R₆ is hydrogen or methyl;
   R₇ is hydrogen;
or an enantiomer, diastereomer, solvate, or pharmaceutically acceptable salt form thereof.

Additional embodiments of the invention include pharmaceutical formulations as described herein, wherein the active pharmaceutical ingredient is a compound of Formula (I) selected from the group consisting of:
N-(2-cyanopyridin-4-yl)-1-(naphthalen-1-yl)-5-(trifluoromethyl)-1H-pyrazole-4-carboxamide;
N-(5-chloro-6-(2H-1,2,3-triazol-2-yl)pyridin-3-yl)-1-(naphthalen-1-yl)-5-(trifluoromethyl)-1H-pyrazole-4-carboxamide;
1-(naphthalen-1-yl)-5-(trifluoromethyl)-N-(2-(trifluoromethyl)pyridin-4-yl)-1H-pyrazole-4-carboxamide;
1-(naphthalen-1-yl)-5-(trifluoromethyl)-N-(5-(trifluoromethyl)pyridin-3-yl)-1H-pyrazole-4-carboxamide;
N-(5-cyanopyridin-3-yl)-1-(naphthalen-1-yl)-5-(trifluoromethyl)-1H-pyrazole-4-carboxamide;
1-(quinolin-5-yl)-5-(trifluoromethyl)-N-(2-(trifluoromethyl)pyridin-4-yl)-1H-pyrazole-4-carboxamide;
N-(5-chloro-6-methoxypyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(3-methylisoquinolin-1-yl)-5-(trifluoromethyl)-1H-pyrazole-4-carboxamide;
N-(3-chloro-4-methoxyphenyl)-1-(isoquinolin-8-yl)-5-(trifluoromethyl)-1H-pyrazole-4-carboxamide;
N-(3-chloro-4-(2H-1,2,3-triazol-2-yl)phenyl)-1-(isoquinolin-8-yl)-5-(trifluoromethyl)-1H-pyrazole-4-carboxamide;
N-(3-chloro-4-(1H-pyrazol-1-yl)phenyl)-1-(isoquinolin-8-yl)-5-(trifluoromethyl)-1H-pyrazole-4-carboxamide;
N-(6-cyano-5-(trifluoromethyl)pyridin-3-yl)-1-(isoquinolin-8-yl)-5-(trifluoromethyl)-1H-pyrazole-4-carboxamide;
N-(4-(2-aminopyrimidin-4-yl)-3-chlorophenyl)-1-(isoquinolin-8-yl)-5-(trifluoromethyl)-1H-pyrazole-4-carboxamide;
N-(5-chloro-6-(1H-pyrazol-1-yl)pyridin-3-yl)-1-(isoquinolin-8-yl)-5-(trifluoromethyl)-1H-pyrazole-4-carboxamide;
N-(5-chloro-6-(2H-1,2,3-triazol-2-yl)pyridin-3-yl)-5-isobutyl-1-(quinolin-5-yl)-1H-pyrazole-4-carboxamide;
N-(5-chloro-6-(2H-1,2,3-triazol-2-yl)pyridin-3-yl)-5-ethyl-1-(quinolin-5-yl)-H-pyrazole-4-carboxamide;
N-(3-chloro-4-(1H-1,2,3-triazol-1-yl)phenyl)-1-(isoquinolin-8-yl)-5-(trifluoromethyl)-1H-pyrazole-4-carboxamide;
N-(5-cyano-6-(2H-1,2,3-triazol-2-yl)pyridin-3-yl)-1-(isoquinolin-8-yl)-5-(trifluoromethyl)-1H-pyrazole-4-carboxamide;
N-(5-chloro-6-(1,1-dioxidoisothiazolidin-2-yl)pyridin-3-yl)-1-(isoquinolin-8-yl)-5-(trifluoromethyl)-1H-pyrazole-4-carboxamide;
N-(5-chloro-6-(1-methyl-1H-pyrazol-3-yl)pyridin-3-yl)-1-(isoquinolin-8-yl)-5-(trifluoromethyl)-1H-pyrazole-4-carboxamide;
N-(5-chloro-6-(oxazol-2-yl)pyridin-3-yl)-1-(isoquinolin-8-yl)-5-(trifluoromethyl)-1H-pyrazole-4-carboxamide;
N-(5-chloro-6-methoxypyridin-3-yl)-1-(isoquinolin-4-yl)-5-(trifluoromethyl)-1H-pyrazole-4-carboxamide;
N-(5-cyano-6-methoxypyridin-3-yl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-1-(3-fluoroquinolin-5-yl)-5-(trifluoromethyl)-1H-pyrazole-4-carboxamide;
N-(5-chloro-6-(2H-1,2,3-triazol-2-yl)pyridin-3-yl)-5-isopropyl-1-(quinolin-5-yl)-1H-pyrazole-4-carboxamide;
N-(5-chloro-6-(2H-1,2,3-triazol-2-yl)pyridin-3-yl)-1-(6-methylquinolin-5-yl)-5-(trifluoromethyl)-1H-pyrazole-4-carboxamide;
N-(5-chloro-6-(2H-1,2,3-triazol-2-yl)pyridin-3-yl)-1-(8-methylquinolin-5-yl)-5-(trifluoromethyl)-1H-pyrazole-4-carboxamide;
N-(3-chloro-4-(3-methyl-1H-1,2,4-triazol-1-yl)phenyl)-1-(isoquinolin-8-yl)-5-(trifluoromethyl)-1H-pyrazole-4-carboxamide;
N-(5-chloro-6-(3-methyl-1H-1,2,4-triazol-1-yl)pyridin-3-yl)-1-(isoquinolin-8-yl)-5-(trifluoromethyl)-1H-pyrazole-4-carboxamide;
N-(3-chloro-4-(5-methyl-1H-1,2,4-triazol-1-yl)phenyl)-1-(isoquinolin-8-yl)-5-(trifluoromethyl)-1H-pyrazole-4-carboxamide;
N-(5-chloro-6-(2H-1,2,3-triazol-2-yl)pyridin-3-yl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-1-(4-methylisoquinolin-8-yl)-5-(trifluoromethyl)-1H-pyrazole-4-carboxamide;
1-(benzofuran-4-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-5-(1-methoxyethyl)-1-(quinolin-5-yl)-1H-pyrazole-4-carboxamide;
N-(5-chloro-6-(2H-1,2,3-triazol-2-yl)pyridin-3-yl)-1-(6-methylisoquinolin-4-yl)-5-(trifluoromethyl)-1H-pyrazole-4-carboxamide;
N-(5-chloro-6-(2H-1,2,3-triazol-2-yl)pyridin-3-yl)-1-(2-methylquinolin-5-yl)-5-(trifluoromethyl)-1H-pyrazole-4-carboxamide;
N-(3-chloro-4-(2H-1,2,3-triazol-2-yl)phenyl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-5-methyl-1-(quinolin-5-yl)-1H-pyrazole-4-carboxamide;
N-(5-chloro-6-(2H-1,2,3-triazol-2-yl)pyridin-3-yl)-1-(8-fluoroquinolin-5-yl)-5-(trifluoromethyl)-1H-pyrazole-4-carboxamide;
N-(6-cyano-5-fluoropyridin-3-yl)-1-(isoquinolin-4-yl)-5-(trifluoromethyl)-1H-pyrazole-4-carboxamide;
N-(5-chloro-6-(1,1-dioxidoisothiazolidin-2-yl)pyridin-3-yl)-1-(isoquinolin-4-yl)-5-(trifluoromethyl)-1H-pyrazole-4-carboxamide;
N-(3-chloro-4-(1H-1,2,3-triazol-1-yl)phenyl)-1-(isoquinolin-4-yl)-5-(trifluoromethyl)-1H-pyrazole-4-carboxamide;
methyl 3-chloro-5-(3-chloro-5-(1-(isoquinolin-4-yl)-5-(trifluoromethyl)-1H-pyrazole-4-carboxamido)picolinamido)picolinate;
N-(5-chloro-6-((1-methylpiperidin-4-yl)oxy)pyridin-3-yl)-1-(isoquinolin-4-yl)-5-(trifluoromethyl)-1H-pyrazole-4-carboxamide;
N-(5-chloro-6-(1H-pyrazol-1-yl)pyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(isoquinolin-4-yl)-5-(trifluoromethyl)-1H-pyrazole-4-carboxamide;
N-(5-chloro-6-(4-methylpiperazine-1-carbonyl)pyridin-3-yl)-1-(isoquinolin-4-yl)-5-(trifluoromethyl)-1H-pyrazole-4-carboxamide;
N-(6-cyano-5-(trifluoromethyl)pyridin-3-yl)-1-(isoquinolin-4-yl)-5-(trifluoromethyl)-1H-pyrazole-4-carboxamide;
N-(5-chloro-6-(oxazol-2-yl)pyridin-3-yl)-1-(isoquinolin-4-yl)-5-(trifluoromethyl)-1H-pyrazole-4-carboxamide;
N-(3-chloro-4-(5-methyl-1H-1,2,4-triazol-1-yl)phenyl)-1-(isoquinolin-4-yl)-5-(trifluoromethyl)-1H-pyrazole-4-carboxamide;
N-(5-chloro-6-(1-methyl-1H-pyrazol-3-yl)pyridin-3-yl)-1-(isoquinolin-4-yl)-5-(trifluoromethyl)-1H-pyrazole-4-carboxamide;
N-(5-chloro-6-(3-methyl-1H-1,2,4-triazol-1-yl)pyridin-3-yl)-1-(isoquinolin-4-yl)-5-(trifluoromethyl)-1H-pyrazole-4-carboxamide;
N-(5-chloro-6-(5-methyl-1H-1,2,4-triazol-1-yl)pyridin-3-yl)-1-(isoquinolin-4-yl)-5-(trifluoromethyl)-1H-pyrazole-4-carboxamide;
N-(3-chloro-4-(3-methyl-1H-1,2,4-triazol-1-yl)phenyl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-5-(difluoromethyl)-1-(isoquinolin-1-yl)-1H-pyrazole-4-carboxamide;
N-(5-chloro-6-(2H-1,2,3-triazol-2-yl)pyridin-3-yl)-1-(isoquinolin-1-yl)-5-(trifluoromethyl)-1H-pyrazole-4-carboxamide;
N-(4-(2-aminopyrimidin-4-yl)-3-chlorophenyl)-1-(isoquinolin-4-yl)-5-(trifluoromethyl)-1H-pyrazole-4-carboxamide;
N-(3-cyano-4-(2H-1,2,3-triazol-2-yl)phenyl)-1-(isoquinolin-4-yl)-5-(trifluoromethyl)-1H-pyrazole-4-carboxamide;
N-(5-fluoro-6-(2H-1,2,3-triazol-2-yl)pyridin-3-yl)-1-(isoquinolin-4-yl)-5-(trifluoromethyl)-1H-pyrazole-4-carboxamide;
N-(3-cyano-4-(1H-1,2,3-triazol-1-yl)phenyl)-1-(isoquinolin-4-yl)-5-(trifluoromethyl)-1H-pyrazole-4-carboxamide;
N-(5-chloro-6-(thiazol-2-yl)pyridin-3-yl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-5-methyl-1-(quinolin-4-yl)-1H-pyrazole-4-carboxamide;
N-(5-chloro-6-(2H-1,2,3-triazol-2-yl)pyridin-3-yl)-1-(3-methylquinolin-5-yl)-5-(trifluoromethyl)-1H-pyrazole-4-carboxamide;
N-(5-chloro-6-(2H-1,2,3-triazol-2-yl)pyridin-3-yl)-1-(1-methylisoquinolin-4-yl)-5-(trifluoromethyl)-1H-pyrazole-4-carboxamide;
N-(5-chloro-6-(2H-1,2,3-triazol-2-yl)pyridin-3-yl)-1-(6-fluoroquinolin-7-yl)-5-(trifluoromethyl)-1H-pyrazole-4-carboxamide;
N-(5-chloro-6-(2H-1,2,3-triazol-2-yl)pyridin-3-yl)-1-(1H-indazol-4-yl)-5-(trifluoromethyl)-1H-pyrazole-4-carboxamide;
N-(5-chloro-6-(1,3,4-oxadiazol-2-yl)pyridin-3-yl)-1-(isoquinolin-4-yl)-5-(trifluoromethyl)-1H-pyrazole-4-carboxamide;
N-(5-chloro-6-(1H-imidazol-1-yl)pyridin-3-yl)-1-(isoquinolin-4-yl)-5-(trifluoromethyl)-1H-pyrazole-4-carboxamide;
N-(6-(2H-1,2,3-triazol-2-yl)pyridin-3-yl)-1-(isoquinolin-4-yl)-5-(trifluoromethyl)-1H-pyrazole-4-carboxamide;
N-(4-aminobutyl)-3-chloro-5-(1-(isoquinolin-4-yl)-5-(trifluoromethyl)-1H-pyrazole-4-carboxamido)picolinamide;
1-(isoquinolin-4-yl)-N-(2-methyl-6-(trifluoromethyl)pyridin-4-yl)-5-(trifluoromethyl)-1H-pyrazole-4-carboxamide;
methyl 6-chloro-4-(1-(isoquinolin-4-yl)-5-(trifluoromethyl)-1H-pyrazole-4-carboxamido)picolinate;
methyl 4-(1-(isoquinolin-4-yl)-5-(trifluoromethyl)-1H-pyrazole-4-carboxamido)picolinate
N-(2-cyanopyridin-4-yl)-1-(isoquinolin-4-yl)-5-(trifluoromethyl)-1H-pyrazole-4-carboxamide;
N-(5-cyano-6-(1-methyl-1H-pyrazol-3-yl)pyridin-3-yl)-1-(isoquinolin-4-yl)-5-(trifluoromethyl)-1H-pyrazole-4-carboxamide;
N-(5-chloro-6-cyclopropoxypyridin-3-yl)-1-(quinolin-5-yl)-5-(trifluoromethyl)-1H-pyrazole-4-carboxamide;
N-(5-cyano-6-((1-methylpiperidin-4-yl)oxy)pyridin-3-yl)-1-(isoquinolin-4-yl)-5-(trifluoromethyl)-1H-pyrazole-4-carboxamide;
N-(5-cyano-6-ethoxypyridin-3-yl)-1-(quinolin-5-yl)-5-(trifluoromethyl)-1H-pyrazole-4-carboxamide;
N-(5-cyanopyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(quinolin-5-yl)-5-(trifluoromethyl)-1H-pyrazole-4-carboxamide;
N-(6-(4-aminobutoxy)-5-cyanopyridin-3-yl)-1-(isoquinolin-4-yl)-5-(trifluoromethyl)-1H-pyrazole-4-carboxamide;
N-(5-cyano-6-methoxypyridin-3-yl)-1-(quinolin-5-yl)-5-(trifluoromethyl)-1H-pyrazole-4-carboxamide;
N-(5-cyano-6-(1H-1,2,4-triazol-1-yl)pyridin-3-yl)-1-(quinolin-5-yl)-5-(trifluoromethyl)-1H-pyrazole-4-carboxamide;
N-(8-chloro-3-oxo-3,4-dihydro-2H-benzo[b][1,4]oxazin-6-yl)-1-(isoquinolin-4-yl)-5-(trifluoromethyl)-1H-pyrazole-4-carboxamide;
N-(5-cyano-6-cyclopropoxypyridin-3-yl)-1-(quinolin-5-yl)-5-(trifluoromethyl)-1H-pyrazole-4-carboxamide;
N-(5-cyano-6-(1-methyl-1H-pyrazol-3-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(thieno[3,2-c]pyridin-4-yl)-5-(trifluoromethyl)-1H-pyrazole-4-carboxamide;
N-(5-chloro-6-(oxazol-2-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroquinolin-4-yl)-5-(trifluoromethyl)-1H-pyrazole-4-carboxamide;
1-(cinnolin-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(furo[3,2-c]pyridin-4-yl)-5-(trifluoromethyl)-1H-pyrazole-4-carboxamide;
N-(8-chloro-4-methyl-3-oxo-3,4-dihydro-2H-benzo[b][1,4]oxazin-6-yl)-1-(quinolin-5-yl)-5-(trifluoromethyl)-1H-pyrazole-4-carboxamide;
N-(5-cyano-6-(4-methylpiperazine-1-carbonyl)pyridin-3-yl)-1-(isoquinolin-4-yl)-5-(trifluoromethyl)-1H-pyrazole-4-carboxamide;
N-(8-chloro-4-methyl-3-oxo-3,4-dihydro-2H-benzo[b][1,4]oxazin-6-yl)-1-(isoquinolin-4-yl)-5-(trifluoromethyl)-1H-pyrazole-4-carboxamide;
N-(5-chloro-6-(1-methyl-1H-imidazol-2-yl)pyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(furo[2,3-c]pyridin-7-yl)-5-(trifluoromethyl)-1H-pyrazole-4-carboxamide;
N-(5-cyano-6-(2H-1,2,3-triazol-2-yl)pyridin-3-yl)-1-(1,6-naphthyridin-5-yl)-5-(trifluoromethyl)-1H-pyrazole-4-carboxamide;
N-(6-(4-(4-aminobutyl)piperazine-1-carbonyl)-5-cyanopyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(phthalazin-1-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyrazin-8-yl)-5-(trifluoromethyl)-1H-pyrazole-4-carboxamide;
N-(5-chloro-6-(1H-imidazol-2-yl)pyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(quinoxalin-5-yl)-5-(trifluoromethyl)-1H-pyrazole-4-carboxamide;
N-(2-methyl-1-oxo-1,2,3,4-tetrahydroisoquinolin-7-yl)-1-(quinolin-5-yl)-5-(trifluoromethyl)-1H-pyrazole-4-carboxamide;
tert-butyl 2-(5-(4-((5-chloro-6-(2H-1,2,3-triazol-2-yl)pyridin-3-yl)carbamoyl)-5-(trifluoromethyl)-1H-pyrazol-1-yl)isoquinolin-1-yl)azetidine-1-carboxylate;
N-(3-(methylsulfonyl)-4-(1H-1,2,3-triazol-1-yl)phenyl)-1-(quinolin-5-yl)-5-(trifluoromethyl)-1H-pyrazole-4-carboxamide;
1-(1,5-bis(tetrahydrofuran-2-yl)isoquinolin-4-yl)-N-(5-chloro-6-(2H-1,2,3-triazol-2-yl)pyridin-3-yl)-5-(trifluoromethyl)-1H-pyrazole-4-carboxamide;
N-(4-methyl-3-oxo-3,4-dihydro-2H-benzo[b][1,4]oxazin-6-yl)-1-(quinolin-5-yl)-5-(trifluoromethyl)-1H-pyrazole-4-carboxamide;
1-(1-(azetidin-2-yl)isoquinolin-5-yl)-N-(5-chloro-6-(2H-1,2,3-triazol-2-yl)pyridin-3-yl)-5-(trifluoromethyl)-1H-pyrazole-4-carboxamide;
N-(3-(methylsulfonyl)-4-(2H-1,2,3-triazol-2-yl)phenyl)-1-(quinolin-5-yl)-5-(trifluoromethyl)-1H-pyrazole-4-carboxamide;
1-(2-(azetidin-2-yl)quinolin-5-yl)-N-(5-chloro-6-(2H-1,2,3-triazol-2-yl)pyridin-3-yl)-5-(trifluoromethyl)-1H-pyrazole-4-carboxamide;
N-(3-oxo-3,4-dihydro-2H-benzo[b][1,4]oxazin-6-yl)-1-(quinolin-5-yl)-5-(trifluoromethyl)-1H-pyrazole-4-carboxamide;
1-(benzo[d]thiazol-4-yl)-N-(2,5-dimethyl-6-(2H-1,2,3-triazol-2-yl)pyridin-3-yl)-5-(trifluoromethyl)-1H-pyrazole-4-carboxamide;
N-(5-methyl-6-(3-methyl-2-oxo-2,3-dihydro-1H-imidazol-1-yl)pyridin-3-yl)-1-(quinolin-5-yl)-5-(trifluoromethyl)-1H-pyrazole-4-carboxamide;
N-(2,5-diethyl-6-(2H-1,2,3-triazol-2-yl)pyridin-3-yl)-1-(imidazo[1,2-a]pyridin-5-yl)-5-(trifluoromethyl)-1H-pyrazole-4-carboxamide;
N-(5-chloro-6-(2H-1,2,3-triazol-2-yl)pyridin-3-yl)-1-(2-(difluoromethyl)quinolin-5-yl)-5-(trifluoromethyl)-1H-pyrazole-4-carboxamide;
N-(5-cyano-6-(2H-1,2,3-triazol-2-yl)pyridin-3-yl)-1-(2-methylbenzo[d]oxazol-4-yl)-5-(trifluoromethyl)-1H-pyrazole-4-carboxamide;
N-(5-chloro-6-(2H-1,2,3-triazol-2-yl)pyridin-3-yl)-1-(2-methoxyquinolin-5-yl)-5-(trifluoromethyl)-1H-pyrazole-4-carboxamide;
1-(1-aminoisoquinolin-5-yl)-N-(5-cyano-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(2-cyanoquinolin-5-yl)-5-(trifluoromethyl)-1H-pyrazole-4-carboxamide;
1-(2-methylimidazo[1,2-a]pyridin-5-yl)-5-(trifluoromethyl)-N-(2-(trifluoromethyl)pyridin-4-yl)-1H-pyrazole-4-carboxamide;
1-(2-chloroquinolin-4-yl)-N-(5-cyano-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(2-chloroquinolin-5-yl)-5-(trifluoromethyl)-1H-pyrazole-4-carboxamide;
N-(5-chloro-6-(2H-1,2,3-triazol-2-yl)pyridin-3-yl)-1-(2-(tetrahydrofuran-2-yl)quinolin-5-yl)-5-(trifluoromethyl)-1H-pyrazole-4-carboxamide;
5-(4-((5-chloro-6-(2H-1,2,3-triazol-2-yl)pyridin-3-yl)carbamoyl)-5-(trifluoromethyl)-H-pyrazol-1-yl)quinoline-2-carboxamide;
N-(5-chloro-6-(2H-1,2,3-triazol-2-yl)pyridin-3-yl)-1-(1H-pyrazolo[3,4-d]pyrimidin-4-yl)-5-(trifluoromethyl)-1H-pyrazole-4-carboxamide;
N-(5-cyano-6-(2H-1,2,3-triazol-2-yl)pyridin-3-yl)-1-(1,6-naphthyridin-4-yl)-5-(trifluoromethyl)-1H-pyrazole-4-carboxamide;
(*R)—N-(5-chloro-6-(2H-1,2,3-triazol-2-yl)pyridin-3-yl)-1-(1-(1-hydroxyethyl)isoquinolin-5-yl)-5-(trifluoromethyl)-1H-pyrazole-4-carboxamide;
1-(benzo[d]thiazol-4-yl)-N-(5-cyano-2-methyl-4-(2H-1,2,3-triazol-2-yl)phenyl)-5-(trifluoromethyl)-1H-pyrazole-4-carboxamide;
(*R)—N-(5-chloro-6-(2H-1,2,3-triazol-2-yl)pyridin-3-yl)-1-(2-(tetrahydrofuran-2-yl)quinolin-5-yl)-5-(trifluoromethyl)-1H-pyrazole-4-carboxamide;
N-(5-chloro-6-(2-oxopyrrolidin-1-yl)pyridin-3-yl)-1-(isoquinolin-4-yl)-5-(trifluoromethyl)-1H-pyrazole-4-carboxamide;
N-(1-methyl-1H-pyrazolo[3,4-b]pyridin-5-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(2-methyl-1-oxo-1,2-dihydroisoquinolin-5-yl)-5-(trifluoromethyl)-1H-pyrazole-4-carboxamide;
N-(5-chloro-6-(5-cyano-1H-1,2,3-triazol-1-yl)pyridin-3-yl)-1-(quinolin-5-yl)-5-(trifluoromethyl)-1H-pyrazole-4-carboxamide;
2-(2-chloro-4-(1-(quinolin-5-yl)-5-(trifluoromethyl)-1H-pyrazole-4-carboxamido)phenyl)-2H-1,2,3-triazole-4-carboxylic acid;
N-(1H-pyrazolo[3,4-b]pyridin-5-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyridin-8-yl)-5-(trifluoromethyl)-1H-pyrazole-4-carboxamide;
(*S)—N-(5-chloro-6-(2H-1,2,3-triazol-2-yl)pyridin-3-yl)-1-(1-(1-hydroxyethyl)isoquinolin-5-yl)-5-(trifluoromethyl)-1H-pyrazole-4-carboxamide;
N-(2-methylpyridin-4-yl)-1-(1-oxo-1,2-dihydroisoquinolin-5-yl)-5-(trifluoromethyl)-1H-pyrazole-4-carboxamide;
1-(benzo[d][1,2,3]thiadiazol-4-yl)-N-(5-chloro-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(2-methylthieno[3,2-b]pyridin-7-yl)-5-(trifluoromethyl)-1H-pyrazole-4-carboxamide;
1-(1-(azetidin-3-yl)isoquinolin-4-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(imidazo[1,5-a]pyridin-5-yl)-5-(trifluoromethyl)-1H-pyrazole-4-carboxamide;
1-(3-chloro-5-(1-(quinolin-5-yl)-5-(trifluoromethyl)-1H-pyrazole-4-carboxamido)pyridin-2-yl)-1H-1,2,3-triazole-4-carboxylic acid;
N-(5-methoxy-6-(1H-1,2,3-triazol-1-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(2-oxo-1,2-dihydroquinolin-5-yl)-5-(trifluoromethyl)-1H-pyrazole-4-carboxamide;
N-(4-aminobutyl)-3-cyano-5-(1-(isoquinolin-4-yl)-5-(trifluoromethyl)-H-pyrazole-4-carboxamido)picolinamide;
N-(4-(4-(aminomethyl)-1H-pyrazol-1-yl)-3-methylphenyl)-1-(quinolin-5-yl)-5-(trifluoromethyl)-1H-pyrazole-4-carboxamide;
1-(2-aminoquinolin-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(1-methyl-1H-indazol-4-yl)-5-(trifluoromethyl)-1H-pyrazole-4-carboxamide;
1-(1-aminoisoquinolin-5-yl)-N-(5-chloro-6-(2H-1,2,3-triazol-2-yl)pyridin-3-yl)-5-(trifluoromethyl)-1H-pyrazole-4-carboxamide;
N-(5-methyl-6-(1-methyl-1H-tetrazol-5-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(1-methyl-1H-pyrazolo[3,4-b]pyridin-4-yl)-5-(trifluoromethyl)-1H-pyrazole-4-carboxamide;
N-(5-chloro-6-(2H-1,2,3-triazol-2-yl)pyridin-3-yl)-1-(1-ethoxyisoquinolin-4-yl)-5-(trifluoromethyl)-1H-pyrazole-4-carboxamide;
1-(1-(azetidin-2-yl)isoquinolin-4-yl)-N-(5-chloro-6-(2H-1,2,3-triazol-2-yl)pyridin-3-yl)-5-(trifluoromethyl)-1H-pyrazole-4-carboxamide;
1-(2-aminoquinolin-5-yl)-N-(5-chloro-6-(2H-1,2,3-triazol-2-yl)pyridin-3-yl)-5-(trifluoromethyl)-1H-pyrazole-4-carboxamide;
1-(1-acetylisoquinolin-5-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(4-(difluoromethyl)quinolin-5-yl)-5-(trifluoromethyl)-1H-pyrazole-4-carboxamide;
N-(5-chloro-6-(2H-1,2,3-triazol-2-yl)pyridin-3-yl)-1-(1-(pyrrolidin-2-yl)isoquinolin-4-yl)-5-(trifluoromethyl)-1H-pyrazole-4-carboxamide;
N-(5-chloro-6-(difluoromethoxy)pyridin-3-yl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-1-(imidazo[1,2-b]pyridazin-6-yl)-5-(trifluoromethyl)-1H-pyrazole-4-carboxamide;
1-(1-aminoisoquinolin-4-yl)-N-(2,5-dimethyl-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-methoxyisoquinolin-5-yl)-5-(trifluoromethyl)-1H-pyrazole-4-carboxamide;
1-(2-aminoquinolin-5-yl)-N-(5-cyano-6-(2H-1,2,3-triazol-2-yl)pyridin-3-yl)-5-(trifluoromethyl)-1H-pyrazole-4-carboxamide;
2-(2-chloro-4-(1-(quinolin-5-yl)-5-(trifluoromethyl)-1H-pyrazole-4-carboxamido)phenyl)-2H-1,2,3-triazole-4-carboxamide;
N-(5-chloro-6-(2H-1,2,3-triazol-2-yl)pyridin-3-yl)-1-(1-(methylthio)isoquinolin-4-yl)-5-(trifluoromethyl)-1H-pyrazole-4-carboxamide;
N-(5-chloro-6-(2H-1,2,3-triazol-2-yl)pyridin-3-yl)-3-fluoro-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(2-methylbenzo[d]thiazol-4-yl)-5-(trifluoromethyl)-1H-pyrazole-4-carboxamide;
1-(3-chloro-5-(1-(quinolin-5-yl)-5-(trifluoromethyl)-1H-pyrazole-4-carboxamido)pyridin-2-yl)-1H-1,2,3-triazole-4-carboxamide;
N-(5-chloro-6-(2H-1,2,3-triazol-2-yl)pyridin-3-yl)-5-cyano-1-(quinolin-5-yl)-1H-pyrazole-4-carboxamide;
1-(7-methylpyrazolo[1,5-a]pyridin-4-yl)-5-(trifluoromethyl)-N-(2-(trifluoromethyl)pyridin-4-yl)-1H-pyrazole-4-carboxamide;
N-(6-(2H-[1,2,3]triazolo[4,5-c]pyridin-2-yl)-5-chloropyridin-3-yl)-1-(quinolin-5-yl)-5-(trifluoromethyl)-1H-pyrazole-4-carboxamide;
1-(2-acetylquinolin-4-yl)-N-(5-chloro-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(2-methylbenzo[d]thiazol-7-yl)-5-(trifluoromethyl)-1H-pyrazole-4-carboxamide;
N-(6-(5-(aminomethyl)-1H-1,2,3-triazol-1-yl)-5-chloropyridin-3-yl)-1-(quinolin-5-yl)-5-(trifluoromethyl)-1H-pyrazole-4-carboxamide;
1-(2-(azetidin-2-yl)quinolin-4-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-(1-ethoxyethyl)isoquinolin-4-yl)-5-(trifluoromethyl)-1H-pyrazole-4-carboxamide;
methyl 1-(3-chloro-5-(1-(quinolin-5-yl)-5-(trifluoromethyl)-1H-pyrazole-4-carboxamido)pyridin-2-yl)-1H-1,2,3-triazole-4-carboxylate
1-(imidazo[1,2-a]pyridin-5-yl)-5-(trifluoromethyl)-N-(2-(trifluoromethyl)pyridin-4-yl)-1H-pyrazole-4-carboxamide;
N-(5-chloro-2-ethyl-6-(2H-1,2,3-triazol-2-yl)pyridin-3-yl)-1-(imidazo[1,2-a]pyridin-5-yl)-5-(trifluoromethyl)-1H-pyrazole-4-carboxamide;
N-(5-chloro-6-(2H-1,2,3-triazol-2-yl)pyridin-3-yl)-1-(2-(difluoromethyl)thieno[2,3-c]pyridin-4-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyrimidin-5-yl)-5-(trifluoromethyl)-1H-pyrazole-4-carboxamide;
N-(5-methoxy-6-(2H-1,2,3-triazol-2-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(2-methoxyquinolin-4-yl)-5-(trifluoromethyl)-1H-pyrazole-4-carboxamide;
1-(benzo[d][1,2,3]thiadiazol-7-yl)-N-(5-chloro-2-methyl-6-(1H-pyrazol-1-yl)pyridin-3-yl)-5-(trifluoromethyl)-1H-pyrazole-4-carboxamide;
N-(2,5-dimethyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(benzo[d]thiazol-4-yl)-N-(5-chloro-2-methyl-6-(2H-1,2,3-triazol-2-yl)pyridin-3-yl)-5-(trifluoromethyl)-1H-pyrazole-4-carboxamide;
N-(5-chloro-6-(5-(methoxymethyl)-1H-1,2,3-triazol-1-yl)pyridin-3-yl)-1-(quinolin-5-yl)-5-(trifluoromethyl)-1H-pyrazole-4-carboxamide;
N-(6-(2H-[1,2,3]triazolo[4,5-b]pyridin-2-yl)-5-chloropyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(2-methyl-[1,2,4]triazolo[1,5-a]pyridin-5-yl)-5-(trifluoromethyl)-1H-pyrazole-4-carboxamide;
3-(3-cyano-5-(1-(quinolin-5-yl)-5-(trifluoromethyl)-1H-pyrazole-4-carboxamido)pyridin-2-yl)-1-methyl-1H-pyrazole-5-carboxylic acid;
1-(benzo[d]thiazol-4-yl)-N-(5-cyano-2-methyl-6-(2H-1,2,3-triazol-2-yl)pyridin-3-yl)-5-(trifluoromethyl)-1H-pyrazole-4-carboxamide;
1-(1-aminoisoquinolin-4-yl)-N-(5-chloro-2-methyl-6-(1-methyl-1H-pyrazol-3-yl)pyridin-3-yl)-5-(trifluoromethyl)-1H-pyrazole-4-carboxamide;
N-(6-(3H-[1,2,3]triazolo[4,5-b]pyridin-3-yl)-5-chloropyridin-3-yl)-1-(quinolin-5-yl)-5-(trifluoromethyl)-1H-pyrazole-4-carboxamide;
methyl 2-(2-chloro-4-(1-(quinolin-5-yl)-5-(trifluoromethyl)-1H-pyrazole-4-carboxamido)phenyl)-2H-1,2,3-triazole-4-carboxylate;
N-(5-cyano-6-(2-methyl-2H-1,2,3-triazol-4-yl)pyridin-3-yl)-1-(quinolin-5-yl)-5-(trifluoromethyl)-1H-pyrazole-4-carboxamide;
N-(3-chloro-4-(5-oxo-4,5-dihydro-1H-1,2,4-triazol-3-yl)phenyl)-1-(quinolin-5-yl)-5-(trifluoromethyl)-1H-pyrazole-4-carboxamide;
methyl 3-chloro-5-(1-(quinolin-5-yl)-5-(trifluoromethyl)-1H-pyrazole-4-carboxamido)picolinate;
N-(6-(1H-[1,2,3]triazolo[4,5-c]pyridin-1-yl)-5-chloropyridin-3-yl)-1-(quinolin-5-yl)-5-(trifluoromethyl)-1H-pyrazole-4-carboxamide;
N-(5-cyanopyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(1-methyl-1H-pyrazolo[3,4-c]pyridin-7-yl)-5-(trifluoromethyl)-1H-pyrazole-4-carboxamide;
1-(1-aminoisoquinolin-4-yl)-N-(5-chloro-2-methyl-6-(1H-pyrazol-1-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-(dimethylamino)isoquinolin-4-yl)-5-(trifluoromethyl)-1H-pyrazole-4-carboxamide;
N-(5-chloro-6-(2H-1,2,3-triazol-2-yl)pyridin-3-yl)-1-(8-(difluoromethyl)quinolin-5-yl)-5-(trifluoromethyl)-1H-pyrazole-4-carboxamide;
(*R)—N-(5-chloro-6-(2H-1,2,3-triazol-2-yl)pyridin-3-yl)-1-(1-(4-oxotetrahydrofuran-2-yl)isoquinolin-4-yl)-5-(trifluoromethyl)-1H-pyrazole-4-carboxamide;
N-(5-cyano-6-(2H-1,2,3-triazol-2-yl)pyridin-3-yl)-1-(2-methylfuro[3,2-b]pyridin-7-yl)-5-(trifluoromethyl)-1H-pyrazole-4-carboxamide;
N-(5-chloro-6-(2H-1,2,3-triazol-2-yl)pyridin-3-yl)-1-(1-(difluoromethyl)isoquinolin-5-yl)-5-(trifluoromethyl)-1H-pyrazole-4-carboxamide;
N-(5-cyano-6-(2H-1,2,3-triazol-2-yl)pyridin-3-yl)-1-(1-methyl-1H-indazol-7-yl)-5-(trifluoromethyl)-1H-pyrazole-4-carboxamide;
N-(5-chloro-6-(2H-1,2,3-triazol-2-yl)pyridin-3-yl)-1-(6-fluoroimidazo[1,2-a]pyridin-5-yl)-5-(trifluoromethyl)-1H-pyrazole-4-carboxamide;
N-(5-chloro-6-(2H-1,2,3-triazol-2-yl)pyridin-3-yl)-1-(5-fluoroquinolin-8-yl)-5-(trifluoromethyl)-1H-pyrazole-4-carboxamide;
N-(5-cyano-6-(2H-1,2,3-triazol-2-yl)pyridin-3-yl)-1-(1H-pyrazolo[4,3-b]pyridin-7-yl)-5-(trifluoromethyl)-1H-pyrazole-4-carboxamide;
N-(5-cyano-6-(2H-1,2,3-triazol-2-yl)pyridin-3-yl)-1-(4-fluoroisoquinolin-1-yl)-5-(trifluoromethyl)-1H-pyrazole-4-carboxamide;
5-(4-((5-chloro-6-(2H-1,2,3-triazol-2-yl)pyridin-3-yl)carbamoyl)-5-(trifluoromethyl)-1H-pyrazol-1-yl)isoquinoline-1-carboxamide;
N-(5-chloro-6-(2H-1,2,3-triazol-2-yl)pyridin-3-yl)-1-(1-(methylamino)isoquinolin-4-yl)-5-(trifluoromethyl)-1H-pyrazole-4-carboxamide;
(*S)—N-(5-chloro-6-(2H-1,2,3-triazol-2-yl)pyridin-3-yl)-1-(1-(1-hydroxyethyl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(1-(1,1-difluoroethyl)isoquinolin-4-yl)-5-(trifluoromethyl)-1H-pyrazole-4-carboxamide;
5-chloro-N-(5-chloro-6-(2H-1,2,3-triazol-2-yl)pyridin-3-yl)-1-(quinolin-5-yl)-1H-pyrazole-4-carboxamide;
1-(1-aminoisoquinolin-4-yl)-N-(5-chloro-2-methyl-4-(2H-1,2,3-triazol-2-yl)phenyl)-5-(trifluoromethyl)-1H-pyrazole-4-carboxamide;
N-(5-chloro-6-(2H-1,2,3-triazol-2-yl)pyridin-3-yl)-1-(1-methoxyisoquinolin-4-yl)-5-(trifluoromethyl)-1H-pyrazole-4-carboxamide;
(*S)—N-(5-chloro-6-(2H-1,2,3-triazol-2-yl)pyridin-3-yl)-1-(1-(4-oxotetrahydrofuran-2-yl)isoquinolin-4-yl)-5-(trifluoromethyl)-1H-pyrazole-4-carboxamide;
(*S)—N-(5-chloro-6-(2H-1,2,3-triazol-2-yl)pyridin-3-yl)-1-(1-(tetrahydrofuran-2-yl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(1-fluoroisoquinolin-4-yl)-5-(trifluoromethyl)-1H-pyrazole-4-carboxamide;
N-(5-cyano-6-(2H-1,2,3-triazol-2-yl)pyridin-3-yl)-1-(1-methyl-1H-pyrazolo[3,4-b]pyridin-3-yl)-5-(trifluoromethyl)-1H-pyrazole-4-carboxamide;
N-(5-chloro-2-methyl-6-(2H-1,2,3-triazol-2-yl)pyridin-3-yl)-1-(8-fluoroimidazo[1,2-a]pyridin-5-yl)-5-(trifluoromethyl)-1H-pyrazole-4-carboxamide;
N-(5-cyano-6-(2H-1,2,3-triazol-2-yl)pyridin-3-yl)-1-(1-methyl-1H-indazol-3-yl)-5-(trifluoromethyl)-1H-pyrazole-4-carboxamide;
N-(5-chloro-6-(2H-1,2,3-triazol-2-yl)pyridin-3-yl)-1-(1-(5-oxopyrrolidin-2-yl)isoquinolin-4-yl)-5-(trifluoromethyl)-1H-pyrazole-4-carboxamide;
methyl 3-(3-cyano-5-(1-(quinolin-5-yl)-5-(trifluoromethyl)-1H-pyrazole-4-carboxamido)pyridin-2-yl)-1-methyl-1H-pyrazole-5-carboxylate;
N-(5-cyano-6-(1-methyl-1H-pyrazol-3-yl)pyridin-3-yl)-1-(8-fluoroquinolin-5-yl)-5-(trifluoromethyl)-1H-pyrazole-4-carboxamide;
N-(5-cyano-6-(2H-1,2,3-triazol-2-yl)pyridin-3-yl)-1-(1,5-naphthyridin-4-yl)-5-(trifluoromethyl)-1H-pyrazole-4-carboxamide;
N-(5-chloro-6-(5-((dimethylamino)methyl)-1H-1,2,3-triazol-1-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(2-methylbenzo[d]oxazol-7-yl)-5-(trifluoromethyl)-1H-pyrazole-4-carboxamide;
N-(6-(4-(aminomethyl)-2H-1,2,3-triazol-2-yl)-5-chloropyridin-3-yl)-1-(quinolin-5-yl)-5-(trifluoromethyl)-1H-pyrazole-4-carboxamide;
N-(5-chloro-6-(1-methyl-1H-pyrazol-3-yl)pyridin-3-yl)-1-(8-fluoroquinolin-5-yl)-5-(trifluoromethyl)-1H-pyrazole-4-carboxamide;
1-(1-aminoisoquinolin-4-yl)-N-(5-cyano-2-methyl-4-(2H-1,2,3-triazol-2-yl)phenyl)-5-(trifluoromethyl)-1H-pyrazole-4-carboxamide;
1-(benzo[d][1,2,3]thiadiazol-7-yl)-N-(5-chloro-2-fluoro-4-(2H-1,2,3-triazol-2-yl)phenyl)-5-(trifluoromethyl)-1H-pyrazole-4-carboxamide;
1-([1,2,4]triazolo[1,5-a]pyridin-5-yl)-N-(5-cyano-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-((N-methylformamido)methyl)isoquinolin-4-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyrazin-5-yl)-5-(trifluoromethyl)-1H-pyrazole-4-carboxamide;
N-(5-cyano-6-(2H-1,2,3-triazol-2-yl)pyridin-3-yl)-1-(1,7-naphthyridin-4-yl)-5-(trifluoromethyl)-1H-pyrazole-4-carboxamide;
N-(5-chloro-2-methyl-6-(1H-pyrazol-1-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(2-fluoroquinolin-5-yl)-5-(trifluoromethyl)-1H-pyrazole-4-carboxamide;
1-(2-aminobenzo[d]thiazol-7-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(isothiazolo[5,4-b]pyridin-3-yl)-5-(trifluoromethyl)-1H-pyrazole-4-carboxamide;
N-(5-cyano-6-(1H-pyrrol-1-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(1-methoxyisoquinolin-5-yl)-5-(trifluoromethyl)-1H-pyrazole-4-carboxamide;
1-(2-aminobenzo[d]thiazol-7-yl)-N-(5-cyano-6-(2H-1,2,3-triazol-2-yl)pyridin-3-yl)-5-(trifluoromethyl)-1H-pyrazole-4-carboxamide;
N-(6-(1H-1,2,3-triazol-1-yl)-5-(trifluoromethyl)pyridin-3-yl)-1-(quinolin-5-yl)-5-(trifluoromethyl)-1H-pyrazole-4-carboxamide;
1-(benzo[d]isoxazol-3-yl)-N-(5-chloro-6-(2H-1,2,3-triazol-2-yl)pyridin-3-yl)-5-(trifluoromethyl)-1H-pyrazole-4-carboxamide;
1-(1-oxo-1,2-dihydroisoquinolin-5-yl)-5-(trifluoromethyl)-N-(2-(trifluoromethyl)pyridin-4-yl)-1H-pyrazole-4-carboxamide;
N-(5-chloro-6-(2H-1,2,3-triazol-2-yl)pyridin-3-yl)-1-(8-fluoro-1-(methylamino)isoquinolin-4-yl)-5-(trifluoromethyl)-1H-pyrazole-4-carboxamide;
5-bromo-N-(5-chloro-6-(2H-1,2,3-triazol-2-yl)pyridin-3-yl)-1-(quinolin-5-yl)-1H-pyrazole-4-carboxamide;
N-(5-chloro-6-(2H-1,2,3-triazol-2-yl)pyridin-3-yl)-1-(2-cyanoquinolin-4-yl)-5-(trifluoromethyl)-1H-pyrazole-4-carboxamide;
N-(5-chloro-6-(2H-1,2,3-triazol-2-yl)pyridin-3-yl)-1-(1-chloroisoquinolin-4-yl)-5-(trifluoromethyl)-1H-pyrazole-4-carboxamide;
1-(pyrazolo[1,5-a]pyridin-4-yl)-5-(trifluoromethyl)-N-(2-(trifluoromethyl)pyridin-4-yl)-1H-pyrazole-4-carboxamide;
N-(5-chloro-6-(2H-1,2,3-triazol-2-yl)pyridin-3-yl)-1-(2-methylimidazo[1,2-a]pyridin-5-yl)-5-(trifluoromethyl)-1H-pyrazole-4-carboxamide;
1-(1-oxo-1,2-dihydroisoquinolin-5-yl)-5-(trifluoromethyl)-N-(5-(trifluoromethyl)pyridin-3-yl)-1H-pyrazole-4-carboxamide;
1-(1-(1,4-dioxan-2-yl)isoquinolin-4-yl)-N-(5-chloro-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(1H-indazol-7-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)quinoline-2-carboxamide;
N-(5-chloro-6-(5-(hydroxymethyl)-1H-1,2,3-triazol-1-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(1-(4-oxotetrahydrofuran-2-yl)isoquinolin-4-yl)-5-(trifluoromethyl)-1H-pyrazole-4-carboxamide;
N-(6-(4-amino-2H-1,2,3-triazol-2-yl)-5-chloropyridin-3-yl)-1-(benzo[d]thiazol-4-yl)-5-(trifluoromethyl)-1H-pyrazole-4-carboxamide;
1-(2-amino-[1,2,4]triazolo[1,5-a]pyridin-5-yl)-N-(5-cyano-6-(2H-1,2,3-triazol-2-yl)pyridin-3-yl)-5-(trifluoromethyl)-1H-pyrazole-4-carboxamide;
(*R)—N-(5-chloro-6-(2H-1,2,3-triazol-2-yl)pyridin-3-yl)-1-(1-(tetrahydrofuran-2-yl)isoquinolin-4-yl)-5-(trifluoromethyl)-1H-pyrazole-4-carboxamide;
N-(5-bromo-6-(1H-1,2,3-triazol-1-yl)pyridin-3-yl)-1-(quinolin-5-yl)-5-(trifluoromethyl)-1H-pyrazole-4-carboxamide;
N-(5-chloro-6-(oxazol-2-yl)pyridin-3-yl)-1-(8-fluoroquinolin-5-yl)-5-(trifluoromethyl)-1H-pyrazole-4-carboxamide;
N-(5-chloro-6-methoxypyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(1-(difluoromethyl)isoquinolin-4-yl)-5-(trifluoromethyl)-1H-pyrazole-4-carboxamide;
N-(5-cyano-6-(2H-1,2,3-triazol-2-yl)pyridin-3-yl)-1-(1-fluoroisoquinolin-4-yl)-5-(trifluoromethyl)-1H-pyrazole-4-carboxamide;
N-(5-chloro-6-(2H-1,2,3-triazol-2-yl)pyridin-3-yl)-1-(1-(methoxymethyl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(2-(1-hydroxyethyl)quinolin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-methylpyrazolo[1,5-a]pyridin-4-yl)-5-(trifluoromethyl)-1H-pyrazole-4-carboxamide;
1-(1-aminoisoquinolin-4-yl)-N-(5-chloro-2-fluoro-4-(2H-1,2,3-triazol-2-yl)phenyl)-5-(trifluoromethyl)-1H-pyrazole-4-carboxamide;
1-(benzo[d]isothiazol-3-yl)-N-(5-chloro-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(2-fluoroquinolin-5-yl)-5-(trifluoromethyl)-1H-pyrazole-4-carboxamide;
N-(5-chloro-6-(2H-1,2,3-triazol-2-yl)pyridin-3-yl)-1-(isoquinolin-5-yl)-5-(trifluoromethyl)-1H-pyrazole-4-carboxamide;
N-(5-cyano-6-(2H-1,2,3-triazol-2-yl)pyridin-3-yl)-1-(furo[2,3-d]pyrimidin-4-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyridin-3-yl)-5-(trifluoromethyl)-1H-pyrazole-4-carboxamide;
N-(5-chloro-2-methyl-6-(2H-1,2,3-triazol-2-yl)pyridin-3-yl)-1-(imidazo[1,2-a]pyridin-5-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-H-pyrazol-1-yl)thieno[2,3-c]pyridine 6-oxide;
1-(benzo[d][1,2,3]thiadiazol-7-yl)-N-(5-chloro-6-(1-methyl-1H-pyrazol-3-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(thiazolo[5,4-d]pyrimidin-7-yl)-5-(trifluoromethyl)-1H-pyrazole-4-carboxamide;
N-(5-cyano-6-(2H-1,2,3-triazol-2-yl)pyridin-3-yl)-1-(2-methylimidazo[1,2-a]pyridin-3-yl)-5-(trifluoromethyl)-1H-pyrazole-4-carboxamide;
N-(5-cyano-6-methoxypyridin-3-yl)-1-(1-oxo-1,2-dihydroisoquinolin-5-yl)-5-(trifluoromethyl)-1H-pyrazole-4-carboxamide;
N-(5-chloro-2-methyl-6-(1-methyl-1H-pyrazol-3-yl)pyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(quinazolin-4-yl)-5-(trifluoromethyl)-1H-pyrazole-4-carboxamide;
N-(5-chloro-2-methyl-4-(2H-1,2,3-triazol-2-yl)phenyl)-1-(8-fluoroisoquinolin-4-yl)-5-(trifluoromethyl)-1H-pyrazole-4-carboxamide;
N-(5-chloro-2-methyl-4-(2H-1,2,3-triazol-2-yl)phenyl)-1-(1-oxo-1,2-dihydroisoquinolin-5-yl)-5-(trifluoromethyl)-1H-pyrazole-4-carboxamide;
(*R)—N-(5-chloro-6-(2H-1,2,3-triazol-2-yl)pyridin-3-yl)-1-(1-(1-hydroxyethyl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(1-cyanoisoquinolin-4-yl)-5-(trifluoromethyl)-1H-pyrazole-4-carboxamide;
N-(5-chloro-2-fluoro-4-(2H-1,2,3-triazol-2-yl)phenyl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(1-(1-hydroxyethyl)isoquinolin-4-yl)-5-(trifluoromethyl)-1H-pyrazole-4-carboxamide;
N-(5-cyano-2-methyl-4-(2H-1,2,3-triazol-2-yl)phenyl)-1-(1-oxo-1,2-dihydroisoquinolin-5-yl)-5-(trifluoromethyl)-1H-pyrazole-4-carboxamide;
N-(5-chloro-6-(4-methyl-1H-1,2,3-triazol-1-yl)pyridin-3-yl)-1-(quinolin-5-yl)-5-(trifluoromethyl)-1H-pyrazole-4-carboxamide;
N-(5-methyl-6-(2-methyl-2H-tetrazol-5-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(7-(difluoromethyl)thieno[2,3-c]pyridin-4-yl)-5-(trifluoromethyl)-1H-pyrazole-4-carboxamide;
N-(5-chloro-6-(2H-1,2,3-triazol-2-yl)pyridin-3-yl)-1-(1-(tetrahydrofuran-2-yl)isoquinolin-4-yl)-5-(trifluoromethyl)-1H-pyrazole-4-carboxamide;
N-(5-chloro-6-(4-(methoxymethyl)-2H-1,2,3-triazol-2-yl)pyridin-3-yl)-1-(quinolin-5-yl)-5-(trifluoromethyl)-1H-pyrazole-4-carboxamide;
N-(4-(4-(aminomethyl)-1H-pyrazol-1-yl)-3-chlorophenyl)-1-(quinolin-5-yl)-5-(trifluoromethyl)-1H-pyrazole-4-carboxamide;
1-(1-aminoisoquinolin-4-yl)-N-(5-cyano-2-methyl-6-(2H-1,2,3-triazol-2-yl)pyridin-3-yl)-5-(trifluoromethyl)-1H-pyrazole-4-carboxamide;
1-(benzo[d]thiazol-4-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-hydroxyisoquinolin-4-yl)-5-(trifluoromethyl)-1H-pyrazole-4-carboxamide;
N-(5-chloro-6-(2H-tetrazol-5-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroquinolin-5-yl)-5-(trifluoromethyl)-1H-pyrazole-4-carboxamide;
N-(5-chloro-6-(2H-1,2,3-triazol-2-yl)pyridin-3-yl)-1-(2-(difluoromethyl)quinolin-4-yl)-5-(trifluoromethyl)-1H-pyrazole-4-carboxamide;
N-(5-cyano-6-(2H-1,2,3-triazol-2-yl)pyridin-3-yl)-1-(8-fluoro-1-(methylamino)isoquinolin-4-yl)-5-(trifluoromethyl)-1H-pyrazole-4-carboxamide;
N-(5-chloro-6-(2H-1,2,3-triazol-4-yl)pyridin-3-yl)-1-(quinolin-5-yl)-5-(trifluoromethyl)-1H-pyrazole-4-carboxamide;
N-(5-chloro-2-fluoro-4-(2H-1,2,3-triazol-2-yl)phenyl)-1-(8-fluoroisoquinolin-4-yl)-5-(trifluoromethyl)-1H-pyrazole-4-carboxamide;
N-(5-chloro-6-(2H-1,2,3-triazol-2-yl)pyridin-3-yl)-1-(1-methyl-2-oxo-1,2-dihydroquinolin-4-yl)-5-(trifluoromethyl)-1H-pyrazole-4-carboxamide;
(*R)—N-(5-chloro-6-(2H-1,2,3-triazol-2-yl)pyridin-3-yl)-1-(1-(3-hydroxypyrrolidin-1-yl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(1-(hydroxymethyl)isoquinolin-4-yl)-5-(trifluoromethyl)-1H-pyrazole-4-carboxamide;
N-(5-cyano-6-(2H-1,2,3-triazol-2-yl)pyridin-3-yl)-1-(3-methylthieno[3,2-b]pyridin-7-yl)-5-(trifluoromethyl)-1H-pyrazole-4-carboxamide;
1-(benzo[d]oxazol-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(4-fluoronaphthalen-1-yl)-5-(trifluoromethyl)-1H-pyrazole-4-carboxamide;
N-(3-chloro-4-(4-(hydroxymethyl)-1H-pyrazol-1-yl)phenyl)-1-(quinolin-5-yl)-5-(trifluoromethyl)-1H-pyrazole-4-carboxamide;
N-(5-cyano-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(8-aminoquinolin-5-yl)-N-(5-cyano-6-(2H-1,2,3-triazol-2-yl)pyridin-3-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)quinoline 1-oxide;
N-(5-cyano-6-(4-(hydroxymethyl)-2H-1,2,3-triazol-2-yl)pyridin-3-yl)-1-(quinolin-5-yl)-5-(trifluoromethyl)-1H-pyrazole-4-carboxamide;
methyl 2-cyano-4-(1-(quinolin-5-yl)-5-(trifluoromethyl)-1H-pyrazole-4-carboxamido)benzoate;
N-(6-(5-amino-1H-1,2,3-triazol-1-yl)-5-chloropyridin-3-yl)-1-(1-oxo-1,2-dihydroisoquinolin-5-yl)-5-(trifluoromethyl)-1H-pyrazole-4-carboxamide;
N-(5-chloro-6-(4-cyano-2H-1,2,3-triazol-2-yl)pyridin-3-yl)-1-(quinolin-5-yl)-5-(trifluoromethyl)-1H-pyrazole-4-carboxamide;
1-(1-aminoisoquinolin-4-yl)-N-(5-chloro-2-methyl-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyridin-5-yl)-5-(trifluoromethyl)-1H-pyrazole-4-carboxamide;
1-(benzo[d][1,2,3]thiadiazol-7-yl)-N-(5-cyano-2-methyl-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(8-fluoroimidazo[1,2-a]pyridin-5-yl)-5-(trifluoromethyl)-1H-pyrazole-4-carboxamide;
N-(6-(5-amino-1-methyl-1H-pyrazol-3-yl)-5-cyanopyridin-3-yl)-1-(quinolin-5-yl)-5-(trifluoromethyl)-1H-pyrazole-4-carboxamide;
N-(5-chloro-2-methyl-6-(2H-1,2,3-triazol-2-yl)pyridin-3-yl)-1-(isoquinolin-4-yl)-5-(trifluoromethyl)-1H-pyrazole-4-carboxamide;
N-(5-chloro-6-(4-(hydroxymethyl)-1H-pyrazol-1-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(3-methylimidazo[1,2-a]pyridin-5-yl)-5-(trifluoromethyl)-1H-pyrazole-4-carboxamide;
N-(6-(5-amino-1-methyl-1H-pyrazol-3-yl)-5-chloropyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroimidazo[1,2-a]pyridin-5-yl)-5-(trifluoromethyl)-1H-pyrazole-4-carboxamide;
N-(5-cyano-6-(2H-1,2,3-triazol-2-yl)pyridin-3-yl)-1-(thieno[3,2-d]pyrimidin-4-yl)-5-(trifluoromethyl)-1H-pyrazole-4-carboxamide;
N-(5-chloro-6-(2H-1,2,3-triazol-2-yl)pyridin-3-yl)-1-(8-fluoro-1-hydroxyisoquinolin-4-yl)-5-(trifluoromethyl)-1H-pyrazole-4-carboxamide;
1-(benzo[d]thiazol-4-yl)-N-(5-cyano-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(quinolin-4-yl)-5-(trifluoromethyl)-1H-pyrazole-4-carboxamide;
N-(5-cyano-6-(2H-1,2,3-triazol-2-yl)pyridin-3-yl)-1-(pyrrolo[1,2-a]pyrazin-1-yl)-5-(trifluoromethyl)-1H-pyrazole-4-carboxamide;
N-(6-(2H-1,2,3-triazol-2-yl)-5-(trifluoromethyl)pyridin-3-yl)-1-(quinolin-5-yl)-5-(trifluoromethyl)-1H-pyrazole-4-carboxamide;
1-(benzo[d][1,2,3]thiadiazol-7-yl)-N-(5-chloro-6-(2H-1,2,3-triazol-2-yl)pyridin-3-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)isoquinoline-1-carboxamide;
N-(5-chloro-6-(2H-1,2,3-triazol-2-yl)pyridin-3-yl)-1-(imidazo[1,2-a]pyridin-5-yl)-5-(trifluoromethyl)-1H-pyrazole-4-carboxamide;
N-(5-chloro-6-(2H-1,2,3-triazol-2-yl)pyridin-3-yl)-1-(pyrrolo[1,2-a]pyrazin-1-yl)-5-(trifluoromethyl)-1H-pyrazole-4-carboxamide;
N-(5-cyano-6-(2-methyl-2H-tetrazol-5-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(1,7-naphthyridin-5-yl)-5-(trifluoromethyl)-1H-pyrazole-4-carboxamide;
2-(3-chloro-5-(1-(quinolin-5-yl)-5-(trifluoromethyl)-1H-pyrazole-4-carboxamido)pyridin-2-yl)-2H-1,2,3-triazole-4-carboxamide;
N-(5-chloro-2-methyl-6-(2H-1,2,3-triazol-2-yl)pyridin-3-yl)-1-(pyrazolo[1,5-a]pyridin-4-yl)-5-(trifluoromethyl)-1H-pyrazole-4-carboxamide;
1-(1-aminoisoquinolin-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(thieno[2,3-c]pyridin-7-yl)-5-(trifluoromethyl)-1H-pyrazole-4-carboxamide;
N-(5-cyano-6-(2H-1,2,3-triazol-2-yl)pyridin-3-yl)-1-(thieno[2,3-d]pyrimidin-4-yl)-5-(trifluoromethyl)-1H-pyrazole-4-carboxamide;
N-(5-chloro-6-(1,3,4-oxadiazol-2-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(pyrazolo[1,5-a]pyrazin-4-yl)-5-(trifluoromethyl)-1H-pyrazole-4-carboxamide;
N-(5-chloro-6-(2H-1,2,3-triazol-2-yl)pyridin-3-yl)-1-(7-(1-hydroxyethyl)thieno[2,3-c]pyridin-4-yl)-5-(trifluoromethyl)-1H-pyrazole-4-carboxamide;
N-(5-cyano-6-(2H-1,2,3-triazol-2-yl)pyridin-3-yl)-1-(pyrrolo[2,1-f][1,2,4]triazin-4-yl)-5-(trifluoromethyl)-1H-pyrazole-4-carboxamide;
N-(5-chloro-6-(1-methyl-1H-pyrazol-3-yl)pyridin-3-yl)-1-(1-oxo-1,2-dihydroisoquinolin-5-yl)-5-(trifluoromethyl)-1H-pyrazole-4-carboxamide;
methyl 2-(3-chloro-5-(1-(quinolin-5-yl)-5-(trifluoromethyl)-1H-pyrazole-4-carboxamido)pyridin-2-yl)-2H-1,2,3-triazole-4-carboxylate;
N-(5-chloro-6-(4-((dimethylamino)methyl)-2H-1,2,3-triazol-2-yl)pyridin-3-yl)-1-(quinolin-5-yl)-5-(trifluoromethyl)-1H-pyrazole-4-carboxamide;
N-(5-bromo-6-(2H-1,2,3-triazol-2-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(quinolin-4-yl)-5-(trifluoromethyl)-1H-pyrazole-4-carboxamide;
1-(benzo[d][1,2,3]thiadiazol-7-yl)-N-(5-chloro-2-methyl-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-cyanoisoquinolin-5-yl)-5-(trifluoromethyl)-1H-pyrazole-4-carboxamide;
N-(5-chloro-6-(4-(hydroxymethyl)-2H-1,2,3-triazol-2-yl)pyridin-3-yl)-1-(quinolin-5-yl)-5-(trifluoromethyl)-1H-pyrazole-4-carboxamide;
N-(5-chloro-6-(4-(hydroxymethyl)-1H-pyrazol-1-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(pyrazolo[1,5-a]pyrazin-4-yl)-5-(trifluoromethyl)-1H-pyrazole-4-carboxamide;
N-(5-cyano-2-methyl-6-(2H-1,2,3-triazol-2-yl)pyridin-3-yl)-1-(8-fluoroisoquinolin-4-yl)-5-(trifluoromethyl)-1H-pyrazole-4-carboxamide;
N-(6-(4-amino-2H-1,2,3-triazol-2-yl)-5-chloropyridin-3-yl)-1-(1-oxo-1,2-dihydroisoquinolin-5-yl)-5-(trifluoromethyl)-1H-pyrazole-4-carboxamide;
N-(6-(4-amino-2H-1,2,3-triazol-2-yl)-5-chloropyridin-3-yl)-1-(quinolin-5-yl)-5-(trifluoromethyl)-1H-pyrazole-4-carboxamide;
1-(1-amino-8-fluoroisoquinolin-4-yl)-N-(5-chloro-6-(2H-1,2,3-triazol-2-yl)pyridin-3-yl)-5-(trifluoromethyl)-1H-pyrazole-4-carboxamide;
1-(1-aminoisoquinolin-4-yl)-N-(5-chloro-6-(2H-1,2,3-triazol-2-yl)pyridin-3-yl)-5-(trifluoromethyl)-1H-pyrazole-4-carboxamide;
N-(3-cyano-4-(2H-1,2,3-triazol-2-yl)phenyl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(2-oxo-1,2-dihydroquinolin-4-yl)-5-(trifluoromethyl)-1H-pyrazole-4-carboxamide;
1-(4-aminonaphthalen-1-yl)-N-(5-cyano-6-(2H-1,2,3-triazol-2-yl)pyridin-3-yl)-5-(trifluoromethyl)-1H-pyrazole-4-carboxamide;
1-(1-aminoisoquinolin-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(4-fluoro-2-methoxyphenyl)-5-(trifluoromethyl)-1H-pyrazole-4-carboxamide;
N-(5-chloro-6-(2H-1,2,3-triazol-2-yl)pyridin-3-yl)-1-(2-D-quinolin-5-yl)-5-(trifluoromethyl)-1H-pyrazole-4-carboxamide;
N-(5-cyano-6-(2H-1,2,3-triazol-2-yl)pyridin-3-yl)-1-(2-D-quinolin-5-yl)-5-(trifluoromethyl)-1H-pyrazole-4-carboxamide;
N-(3-chloro-4-(2H-1,2,3-triazol-2-yl)phenyl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(thieno[3,2-b]pyridin-7-yl)-5-(trifluoromethyl)-1H-pyrazole-4-carboxamide;
N-(5-bromo-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(pyrazolo[1,5-a]pyridin-4-yl)-5-(trifluoromethyl)-1H-pyrazole-4-carboxamide;
N-(5-chloro-6-(oxazol-2-yl)pyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroisoquinolin-4-yl)-5-(trifluoromethyl)-1H-pyrazole-4-carboxamide;
N-(5-cyano-6-(2H-1,2,3-triazol-2-yl)pyridin-3-yl)-1-(thieno[2,3-b]pyridin-4-yl)-5-(trifluoromethyl)-1H-pyrazole-4-carboxamide;
N-(5-chloro-6-(2H-1,2,3-triazol-2-yl)pyridin-3-yl)-1-(thieno[2,3-c]pyridin-4-yl)-5-(trifluoromethyl)-1H-pyrazole-4-carboxamide;
N-(5-chloro-6-(4-methyl-2H-1,2,3-triazol-2-yl)pyridin-3-yl)-1-(quinolin-5-yl)-5-(trifluoromethyl)-1H-pyrazole-4-carboxamide;
N-(5-chloro-2-methyl-6-(2H-1,2,3-triazol-2-yl)pyridin-3-yl)-1-(8-fluoroisoquinolin-4-yl)-5-(trifluoromethyl)-1H-pyrazole-4-carboxamide;
1-(benzo[d][1,2,3]thiadiazol-7-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(5-fluoronaphthalen-1-yl)-5-(trifluoromethyl)-1H-pyrazole-4-carboxamide;
N-(5-chloro-6-(2H-1,2,3-triazol-2-yl)pyridin-3-yl)-1-(8-fluoroisoquinolin-4-yl)-5-(trifluoromethyl)-1H-pyrazole-4-carboxamide;
N-(5-chloro-6-(2H-1,2,3-triazol-2-yl)pyridin-3-yl)-1-(1-hydroxyisoquinolin-5-yl)-5-(trifluoromethyl)-1H-pyrazole-4-carboxamide;
1-(benzo[d]thiazol-7-yl)-N-(5-chloro-6-(2H-1,2,3-triazol-2-yl)pyridin-3-yl)-5-(trifluoromethyl)-1H-pyrazole-4-carboxamide;
N-(5-chloro-6-(4-(hydroxymethyl)-2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(benzo[d]thiazol-7-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(1-amino-8-fluoroisoquinolin-4-yl)-N-(5-cyano-6-(2H-1,2,3-triazol-2-yl)pyridin-3-yl)-5-(trifluoromethyl)-1H-pyrazole-4-carboxamide;
2-(3-chloro-5-(1-(quinolin-5-yl)-5-(trifluoromethyl)-1H-pyrazole-4-carboxamido)pyridin-2-yl)-2H-1,2,3-triazole-4-carboxylic acid;
N-(5-cyano-6-(2H-1,2,3-triazol-2-yl)pyridin-3-yl)-1-(furo[3,2-b]pyridin-7-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)thieno[2,3-c]pyridine-7-carboxamide;
1-(7-(3-hydroxyazetidin-1-yl)thieno[2,3-c]pyridin-4-yl)-5-(trifluoromethyl)-N-(2-(trifluoromethyl)pyridin-4-yl)-1H-pyrazole-4-carboxamide;
1-(1-aminoisoquinolin-4-yl)-N-(5-bromo-2-methyl-6-(2H-1,2,3-triazol-2-yl)pyridin-3-yl)-5-(trifluoromethyl)-1H-pyrazole-4-carboxamide;
N-(5-chloro-2-methyl-6-(4-methyl-1H-1,2,3-triazol-1-yl)pyridin-3-yl)-1-(1-oxo-1,2-dihydroisoquinolin-5-yl)-5-(trifluoromethyl)-1H-pyrazole-4-carboxamide;
1-(1-aminoisoquinolin-4-yl)-5-(trifluoromethyl)-N-(5-(trifluoromethyl)pyridin-3-yl)-1H-pyrazole-4-carboxamide;
1-(1-aminoisoquinolin-4-yl)-N-(5-chloro-2-methyl-6-(4-methyl-2H-1,2,3-triazol-2-yl)pyridin-3-yl)-5-(trifluoromethyl)-1H-pyrazole-4-carboxamide;
N-(5-ethynyl-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-2-methyl-6-(4-methyl-2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(2-methyl-1-oxo-1,2-dihydroisoquinolin-4-yl)-5-(trifluoromethyl)-1H-pyrazole-4-carboxamide;
(*R)—N-(5-chloro-6-(2H-1,2,3-triazol-2-yl)pyridin-3-yl)-1-(7-(3-hydroxypyrrolidin-1-yl)thieno[2,3-c]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-chlorothieno[2,3-c]pyridin-4-yl)-5-(trifluoromethyl)-1H-pyrazole-4-carboxamide;
(*S)—N-(5-chloro-6-(2H-1,2,3-triazol-2-yl)pyridin-3-yl)-1-(7-(3-hydroxypyrrolidin-1-yl)thieno[2,3-c]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-cyanothieno[2,3-c]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-(3-hydroxyazetidin-1-yl)thieno[2,3-c]pyridin-4-yl)-5-(trifluoromethyl)-1H-pyrazole-4-carboxamide;
4-(4-((5-chloro-2-methyl-6-(2H-1,2,3-triazol-2-yl)pyridin-3-yl)carbamoyl)-5-(trifluoromethyl)-1H-pyrazol-1-yl)thieno[2,3-c]pyridine-7-carboxamide;
N-(5-chloro-6-(2H-1,2,3-triazol-2-yl)pyridin-3-yl)-1-(7-cyclopropylthieno[2,3-c]pyridin-4-yl)-5-(trifluoromethyl)-1H-pyrazole-4-carboxamide;
N-(5-chloro-6-(2H-1,2,3-triazol-2-yl)pyridin-3-yl)-1-(7-methylthieno[2,3-c]pyridin-4-yl)-5-(trifluoromethyl)-1H-pyrazole-4-carboxamide;
N-(5-chloro-6-(2H-1,2,3-triazol-2-yl)pyridin-3-yl)-1-(7-cyanothieno[2,3-c]pyridin-4-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-H-pyrazol-1-yl)-N-methylthieno[2,3-c]pyridine-7-carboxamide;
N-(5-chloro-6-(2H-1,2,3-triazol-2-yl)pyridin-3-yl)-1-(7-(3-hydroxyazetidin-1-yl)thieno[2,3-c]pyridin-4-yl)-5-(trifluoromethyl)-1H-pyrazole-4-carboxamide;
N-(5-chloro-6-(2H-1,2,3-triazol-2-yl)pyridin-3-yl)-1-(7-chlorothieno[2,3-c]pyridin-4-yl)-5-(trifluoromethyl)-1H-pyrazole-4-carboxamide;
1-(1-aminoisoquinolin-4-yl)-5-(trifluoromethyl)-N-(2-(trifluoromethyl)pyridin-4-yl)-1H-pyrazole-4-carboxamide;
1-(1-aminoisoquinolin-4-yl)-N-(2-cyanopyridin-4-yl)-5-(trifluoromethyl)-1H-pyrazole-4-carboxamide;
N-(5-chloropyridin-3-yl)-1-(1-oxo-1,2-dihydroisoquinolin-5-yl)-5-(trifluoromethyl)-1H-pyrazole-4-carboxamide;
1-(thieno[2,3-c]pyridin-4-yl)-5-(trifluoromethyl)-N-(2-(trifluoromethyl)pyridin-4-yl)-1H-pyrazole-4-carboxamide;
1-(8-fluoroimidazo[1,2-a]pyridin-5-yl)-5-(trifluoromethyl)-N-(2-(trifluoromethyl)pyridin-4-yl)-1H-pyrazole-4-carboxamide;
N-(6-cyano-5-(trifluoromethyl)pyridin-3-yl)-1-(1-oxo-1,2-dihydroisoquinolin-5-yl)-5-(trifluoromethyl)-1H-pyrazole-4-carboxamide;
methyl 3-chloro-5-(1-(1-oxo-1,2-dihydroisoquinolin-5-yl)-5-(trifluoromethyl)-1H-pyrazole-4-carboxamido)picolinate;
1-(8-fluoroisoquinolin-4-yl)-5-(trifluoromethyl)-N-(2-(trifluoromethyl)pyridin-4-yl)-1H-pyrazole-4-carboxamide;
N-(2-cyanopyridin-4-yl)-1-(1-oxo-1,2-dihydroisoquinolin-5-yl)-5-(trifluoromethyl)-1H-pyrazole-4-carboxamide;
N-(2-(2-methoxyethoxy)-5-(trifluoromethyl)pyridin-3-yl)-1-(1-oxo-1,2-dihydroisoquinolin-5-yl)-5-(trifluoromethyl)-1H-pyrazole-4-carboxamide;
N-(5-chloro-2-methyl-6-(2H-1,2,3-triazol-2-yl)pyridin-3-yl)-1-(thieno[2,3-c]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(2-oxo-1,2-dihydroquinolin-4-yl)-5-(trifluoromethyl)-1H-pyrazole-4-carboxamide;
N-(5-chloro-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide; and
(*S)—N-(5-chloro-6-(2H-1,2,3-triazol-2-yl)pyridin-3-yl)-1-(2-(tetrahydrofuran-2-yl)quinolin-5-yl)-5-(trifluoromethyl)-1H-pyrazole-4-carboxamide;
   or an enantiomer, diastereomer, solvate, or a pharmaceutically acceptable salt form thereof.

Additional embodiments of the invention include pharmaceutical formulations as described herein, wherein the active pharmaceutical ingredient is a compound selected from the group consisting of:
N-(5-chloro-6-(2H-1,2,3-triazol-2-yl)pyridin-3-yl)-1-(quinolin-5-yl)-1H-pyrazole-4-carboxamide;
N-(5-chloro-6-(2H-1,2,3-triazol-2-yl)pyridin-3-yl)-1-(isoquinolin-4-yl)-1H-pyrazole-4-carboxamide;
N-(2-methyl-1-oxo-1,2-dihydroisoquinolin-7-yl)-1-(quinolin-5-yl)-5-(trifluoromethyl)-1H-pyrazole-4-carboxamide;
N-(5-cyano-6-(4-methylpiperazin-1-yl)pyridin-3-yl)-1-(quinolin-5-yl)-5-(trifluoromethyl)-1H-pyrazole-4-carboxamide;
2-cyano-4-(1-(quinolin-5-yl)-5-(trifluoromethyl)-1H-pyrazole-4-carboxamido)benzoic acid;
N-(2-morpholinopyridin-4-yl)-1-(1-oxo-1,2-dihydroisoquinolin-5-yl)-5-(trifluoromethyl)-1H-pyrazole-4-carboxamide;
N-(2-methoxypyridin-4-yl)-1-(1-oxo-1,2-dihydroisoquinolin-5-yl)-5-(trifluoromethyl)-1H-pyrazole-4-carboxamide;
N-(6-methyl-5-(trifluoromethyl)pyridin-3-yl)-1-(1-oxo-1,2-dihydroisoquinolin-5-yl)-5-(trifluoromethyl)-1H-pyrazole-4-carboxamide;
1-(1-oxo-1,2-dihydroisoquinolin-5-yl)-N-(pyridin-4-yl)-5-(trifluoromethyl)-1H-pyrazole-4-carboxamide;
N-(2-cyclopropylpyridin-4-yl)-1-(1-oxo-1,2-dihydroisoquinolin-5-yl)-5-(trifluoromethyl)-1H-pyrazole-4-carboxamide;
3-chloro-N,N-dimethyl-5-(1-(1-oxo-1,2-dihydroisoquinolin-5-yl)-5-(trifluoromethyl)-1H-pyrazole-4-carboxamido)picolinamide;
3-chloro-N-methyl-5-(1-(1-oxo-1,2-dihydroisoquinolin-5-yl)-5-(trifluoromethyl)-1H-pyrazole-4-carboxamido)picolinamide;
1-(1-aminoisoquinolin-4-yl)-N-(6-methyl-5-(trifluoromethyl)pyridin-3-yl)-5-(trifluoromethyl)-1H-pyrazole-4-carboxamide;
N-(5-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
   or an enantiomer, diastereomer, solvate or pharmaceutically acceptable salt form thereof.

Additional embodiments of the invention include pharmaceutical formulations as described herein, wherein the active pharmaceutical ingredient is a compound of Formula (I) selected from the group consisting of:
N-(5-chloro-6-(2H-1,2,3-triazol-2-yl)pyridin-3-yl)-1-(naphthalen-1-yl)-5-(trifluoromethyl)-1H-pyrazole-4-carboxamide;
N-(5-chloro-6-(2H-1,2,3-triazol-2-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(isoquinolin-4-yl)-5-(trifluoromethyl)-1H-pyrazole-4-carboxamide;
1-(benzofuran-4-yl)-N-(5-chloro-6-(2H-1,2,3-triazol-2-yl)pyridin-3-yl)-5-(trifluoromethyl)-1H-pyrazole-4-carboxamide;
N-(3-chloro-4-(2H-1,2,3-triazol-2-yl)phenyl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(isoquinolin-4-yl)-5-(trifluoromethyl)-1H-pyrazole-4-carboxamide;
N-(5-chloro-6-(oxazol-2-yl)pyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(thieno[3,2-c]pyridin-4-yl)-5-(trifluoromethyl)-1H-pyrazole-4-carboxamide;
N-(5-chloro-6-(oxazol-2-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroquinolin-4-yl)-5-(trifluoromethyl)-1H-pyrazole-4-carboxamide;
N-(5-cyano-6-(2H-1,2,3-triazol-2-yl)pyridin-3-yl)-1-(furo[3,2-c]pyridin-4-yl)-5-(trifluoromethyl)-1H-pyrazole-4-carboxamide;
N-(5-cyano-6-(2H-1,2,3-triazol-2-yl)pyridin-3-yl)-1-(furo[2,3-c]pyridin-7-yl)-5-(trifluoromethyl)-1H-pyrazole-4-carboxamide;
methyl 2-(3-chloro-5-(1-(quinolin-5-yl)-5-(trifluoromethyl)-1H-pyrazole-4-carboxamido)pyridin-2-yl)-2H-1,2,3-triazole-4-carboxylate;
2-(3-chloro-5-(1-(quinolin-5-yl)-5-(trifluoromethyl)-1H-pyrazole-4-carboxamido)pyridin-2-yl)-2H-1,2,3-triazole-4-carboxylic acid;
1-(1-amino-8-fluoroisoquinolin-4-yl)-N-(5-chloro-6-(2H-1,2,3-triazol-2-yl)pyridin-3-yl)-5-(trifluoromethyl)-1H-pyrazole-4-carboxamide;
1-(1-amino-8-fluoroisoquinolin-4-yl)-N-(5-cyano-6-(2H-1,2,3-triazol-2-yl)pyridin-3-yl)-5-(trifluoromethyl)-1H-pyrazole-4-carboxamide;
N-(5-chloro-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(benzo[d]thiazol-7-yl)-N-(5-cyano-6-(2H-1,2,3-triazol-2-yl)pyridin-3-yl)-5-(trifluoromethyl)-1H-pyrazole-4-carboxamide;
N-(5-chloro-6-(4-(hydroxymethyl)-2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(1-hydroxyisoquinolin-5-yl)-5-(trifluoromethyl)-1H-pyrazole-4-carboxamide;
N-(5-chloro-6-(2H-1,2,3-triazol-2-yl)pyridin-3-yl)-1-(8-fluoroisoquinolin-4-yl)-5-(trifluoromethyl)-1H-pyrazole-4-carboxamide;
N-(5-cyano-6-(2H-1,2,3-triazol-2-yl)pyridin-3-yl)-1-(8-fluoroisoquinolin-4-yl)-5-(trifluoromethyl)-1H-pyrazole-4-carboxamide;
1-(benzo[d][1,2,3]thiadiazol-7-yl)-N-(5-cyano-6-(2H-1,2,3-triazol-2-yl)pyridin-3-yl)-5-(trifluoromethyl)-1H-pyrazole-4-carboxamide;
1-(benzo[d][1,2,3]thiadiazol-7-yl)-N-(5-chloro-6-(2H-1,2,3-triazol-2-yl)pyridin-3-yl)-5-(trifluoromethyl)-1H-pyrazole-4-carboxamide;
1-(benzo[d][1,2,3]thiadiazol-7-yl)-N-(5-chloro-2-methyl-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(5-fluoronaphthalen-1-yl)-5-(trifluoromethyl)-1H-pyrazole-4-carboxamide;
N-(5-chloro-2-methyl-6-(2H-1,2,3-triazol-2-yl)pyridin-3-yl)-1-(8-fluoroisoquinolin-4-yl)-5-(trifluoromethyl)-1H-pyrazole-4-carboxamide;
N-(5-cyano-2-methyl-6-(2H-1,2,3-triazol-2-yl)pyridin-3-yl)-1-(8-fluoroisoquinolin-4-yl)-5-(trifluoromethyl)-1H-pyrazole-4-carboxamide;
N-(5-chloro-6-(4-methyl-2H-1,2,3-triazol-2-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(thieno[2,3-b]pyridin-4-yl)-5-(trifluoromethyl)-1H-pyrazole-4-carboxamide;
N-(5-chloro-6-(oxazol-2-yl)pyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(thieno[3,2-b]pyridin-7-yl)-5-(trifluoromethyl)-1H-pyrazole-4-carboxamide;
N-(3-chloro-4-(2H-1,2,3-triazol-2-yl)phenyl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(2-D-quinolin-5-yl)-5-(trifluoromethyl)-1H-pyrazole-4-carboxamide;
N-(5-chloro-6-(2H-1,2,3-triazol-2-yl)pyridin-3-yl)-1-(2-Dquinolin-5-yl)-5-(trifluoromethyl)-1H-pyrazole-4-carboxamide;
1-(4-aminonaphthalen-1-yl)-N-(5-cyano-6-(2H-1,2,3-triazol-2-yl)pyridin-3-yl)-5-(trifluoromethyl)-1H-pyrazole-4-carboxamide;
N-(3-cyano-4-(2H-1,2,3-triazol-2-yl)phenyl)-1-(quinolin-5-yl)-5-(trifluoromethyl)-1H-pyrazole-4-carboxamide;
N-(6-(4-amino-2H-1,2,3-triazol-2-yl)-5-chloropyridin-3-yl)-1-(1-oxo-1,2-dihydroisoquinolin-5-yl)-5-(trifluoromethyl)-1H-pyrazole-4-carboxamide;
N-(5-chloro-6-(4-(hydroxymethyl)-1H-pyrazol-1-yl)pyridin-3-yl)-1-(quinolin-5-yl)-5-(trifluoromethyl)-1H-pyrazole-4-carboxamide;
N-(5-chloro-6-(4-(hydroxymethyl)-2H-1,2,3-triazol-2-yl)pyridin-3-yl)-1-(quinolin-5-yl)-5-(trifluoromethyl)-1H-pyrazole-4-carboxamide;
N-(5-chloro-6-(4-((dimethylamino)methyl)-2H-1,2,3-triazol-2-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(quinolin-4-yl)-5-(trifluoromethyl)-1H-pyrazole-4-carboxamide;
N-(5-bromo-6-(2H-1,2,3-triazol-2-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(pyrrolo[2,1-f][1,2,4]triazin-4-yl)-5-(trifluoromethyl)-1H-pyrazole-4-carboxamide;
N-(5-chloro-6-(2H-1,2,3-triazol-2-yl)pyridin-3-yl)-1-(pyrazolo[1,5-a]pyrazin-4-yl)-5-(trifluoromethyl)-1H-pyrazole-4-carboxamide;
N-(5-cyano-6-(2H-1,2,3-triazol-2-yl)pyridin-3-yl)-1-(thieno[2,3-d]pyrimidin-4-yl)-5-(trifluoromethyl)-1H-pyrazole-4-carboxamide;
N-(5-cyano-6-(2H-1,2,3-triazol-2-yl)pyridin-3-yl)-1-(thieno[2,3-c]pyridin-7-yl)-5-(trifluoromethyl)-1H-pyrazole-4-carboxamide;
2-(3-chloro-5-(1-(quinolin-5-yl)-5-(trifluoromethyl)-1H-pyrazole-4-carboxamido)pyridin-2-yl)-2H-1,2,3-triazole-4-carboxamide;
N-(5-cyano-6-(2H-1,2,3-triazol-2-yl)pyridin-3-yl)-1-(1,7-naphthyridin-5-yl)-5-(trifluoromethyl)-1H-pyrazole-4-carboxamide;
N-(5-chloro-6-(2H-1,2,3-triazol-2-yl)pyridin-3-yl)-1-(pyrrolo[1,2-a]pyrazin-1-yl)-5-(trifluoromethyl)-1H-pyrazole-4-carboxamide;
N-(5-cyano-6-(2-methyl-2H-tetrazol-5-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(furo[3,2-b]pyridin-7-yl)-5-(trifluoromethyl)-1H-pyrazole-4-carboxamide;
N-(5-cyano-6-(2H-1,2,3-triazol-2-yl)pyridin-3-yl)-1-(4-fluoro-2-methoxyphenyl)-5-(trifluoromethyl)-1H-pyrazole-4-carboxamide;
1-(benzo[d]thiazol-7-yl)-N-(5-chloro-6-(2H-1,2,3-triazol-2-yl)pyridin-3-yl)-5-(trifluoromethyl)-1H-pyrazole-4-carboxamide;
N-(6-(4-amino-2H-1,2,3-triazol-2-yl)-5-chloropyridin-3-yl)-1-(quinolin-5-yl)-5-(trifluoromethyl)-1H-pyrazole-4-carboxamide;
1-(1-aminoisoquinolin-4-yl)-N-(5-chloro-6-(2H-1,2,3-triazol-2-yl)pyridin-3-yl)-5-(trifluoromethyl)-1H-pyrazole-4-carboxamide;
1-(1-aminoisoquinolin-4-yl)-N-(5-cyano-6-(2H-1,2,3-triazol-2-yl)pyridin-3-yl)-5-(trifluoromethyl)-1H-pyrazole-4-carboxamide;
N-(5-chloro-6-(1-methyl-1H-pyrazol-3-yl)pyridin-3-yl)-1-(1-oxo-1,2-dihydroisoquinolin-5-yl)-5-(trifluoromethyl)-1H-pyrazole-4-carboxamide;
1-(1-aminoisoquinolin-4-yl)-N-(5-chloro-2-methyl-6-(2H-1,2,3-triazol-2-yl)pyridin-3-yl)-5-(trifluoromethyl)-1H-pyrazole-4-carboxamide;
N-(5-bromo-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-6-(1,3,4-oxadiazol-2-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(pyrazolo[1,5-a]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(pyrazolo[1,5-a]pyridin-4-yl)-5-(trifluoromethyl)-1H-pyrazole-4-carboxamide;
N-(5-chloro-6-(2H-1,2,3-triazol-2-yl)pyridin-3-yl)-1-(3-chloroimidazo[1,2-a]pyridin-5-yl)-5-(trifluoromethyl)-1H-pyrazole-4-carboxamide;
N-(5-chloro-2-methyl-6-(2H-1,2,3-triazol-2-yl)pyridin-3-yl)-1-(thieno[2,3-c]pyridin-4-yl)-5-(trifluoromethyl)-1H-pyrazole-4-carboxamide;
1-(1-aminoisoquinolin-4-yl)-N-(5-chloro-2-methyl-6-(4-methyl-2H-1,2,3-triazol-2-yl)pyridin-3-yl)-5-(trifluoromethyl)-1H-pyrazole-4-carboxamide;
1-(1-aminoisoquinolin-4-yl)-N-(5-bromo-2-methyl-6-(2H-1,2,3-triazol-2-yl)pyridin-3-yl)-5-(trifluoromethyl)-1H-pyrazole-4-carboxamide;
methyl
3-chloro-5-(1-(1-oxo-1,2-dihydroisoquinolin-5-yl)-5-(trifluoromethyl)-1H-pyrazole-4-carboxamido)picolinate;
N-(5-chloro-2-methyl-6-(2H-1,2,3-triazol-2-yl)pyridin-3-yl)-1-(2-oxo-1,2-dihydroquinolin-4-yl)-5-(trifluoromethyl)-1H-pyrazole-4-carboxamide;
N-(5-chloro-2-methyl-6-(4-methyl-2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(7-(1-hydroxyethyl)thieno[2,3-c]pyridin-4-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)isoquinoline-1-carboxamide;
4-(4-((5-chloro-6-(2H-1,2,3-triazol-2-yl)pyridin-3-yl)carbamoyl)-5-(trifluoromethyl)-1H-pyrazol-1-yl)thieno[2,3-c]pyridine-7-carboxamide;
4-(4-((5-chloro-6-(2H-1,2,3-triazol-2-yl)pyridin-3-yl)carbamoyl)-5-(trifluoromethyl)-1H-pyrazol-1-yl)-N-methylthieno[2,3-c]pyridine-7-carboxamide;
4-(4-((5-chloro-2-methyl-6-(2H-1,2,3-triazol-2-yl)pyridin-3-yl)carbamoyl)-5-(trifluoromethyl)-1H-pyrazol-1-yl)thieno[2,3-c]pyridine-7-carboxamide;
5-(4-((5-chloro-6-(2H-1,2,3-triazol-2-yl)pyridin-3-yl)carbamoyl)-5-(trifluoromethyl)-1H-pyrazol-1-yl)isoquinoline 2-oxide;
N-(5-chloro-6-(2H-1,2,3-triazol-2-yl)pyridin-3-yl)-1-(2-cyanoquinolin-4-yl)-5-(trifluoromethyl)-1H-pyrazole-4-carboxamide;
N-(5-chloro-6-(2H-1,2,3-triazol-2-yl)pyridin-3-yl)-1-(1-cyanoisoquinolin-4-yl)-5-(trifluoromethyl)-1H-pyrazole-4-carboxamide;
N-(5-chloro-6-(2H-1,2,3-triazol-2-yl)pyridin-3-yl)-1-(1-methyl-2-oxo-1,2-dihydroquinolin-4-yl)-5-(trifluoromethyl)-1H-pyrazole-4-carboxamide;
N-(5-chloro-6-(2H-1,2,3-triazol-2-yl)pyridin-3-yl)-1-(2-methoxyquinolin-5-yl)-5-(trifluoromethyl)-1H-pyrazole-4-carboxamide;
N-(5-chloro-6-(2H-1,2,3-triazol-2-yl)pyridin-3-yl)-1-(1-methoxyisoquinolin-4-yl)-5-(trifluoromethyl)-1H-pyrazole-4-carboxamide;
N-(5-chloro-6-(2H-1,2,3-triazol-2-yl)pyridin-3-yl)-1-(1-ethoxyisoquinolin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-chlorothieno[2,3-c]pyridin-4-yl)-5-(trifluoromethyl)-1H-pyrazole-4-carboxamide;
(R)—N-(5-chloro-6-(2H-1,2,3-triazol-2-yl)pyridin-3-yl)-1-(1-(3-hydroxypyrrolidin-1-yl)isoquinolin-4-yl)-5-(trifluoromethyl)-1H-pyrazole-4-carboxamide;
N-(5-cyano-6-methoxypyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(1-(methylthio)isoquinolin-4-yl)-5-(trifluoromethyl)-1H-pyrazole-4-carboxamide;
N-(5-chloro-6-(2H-1,2,3-triazol-2-yl)pyridin-3-yl)-1-(7-(3-hydroxyazetidin-1-yl)thieno[2,3-c]pyridin-4-yl)-5-(trifluoromethyl)-1H-pyrazole-4-carboxamide;
(S)—N-(5-chloro-6-(2H-1,2,3-triazol-2-yl)pyridin-3-yl)-1-(7-(3-hydroxypyrrolidin-1-yl)thieno[2,3-c]pyridin-4-yl)-5-(trifluoromethyl)-1H-pyrazole-4-carboxamide;
(*S)—N-(5-chloro-6-(2H-1,2,3-triazol-2-yl)pyridin-3-yl)-1-(1-(1-hydroxyethyl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(isoquinolin-5-yl)-5-(trifluoromethyl)-1H-pyrazole-4-carboxamide;
N-(5-chloro-6-(2H-1,2,3-triazol-2-yl)pyridin-3-yl)-1-(quinolin-4-yl)-5-(trifluoromethyl)-1H-pyrazole-4-carboxamide;
and
1-(1-oxo-1,2-dihydroisoquinolin-5-yl)-5-(trifluoromethyl)-N-(2-(trifluoromethyl)pyridin-4-yl)-1H-pyrazole-4-carboxamide;
   or an enantiomer, diastereomer, solvate or pharmaceutically acceptable salt form thereof.

In particular, the active pharmaceutical ingredient may be a compound of Formula (I) selected from the group consisting of:

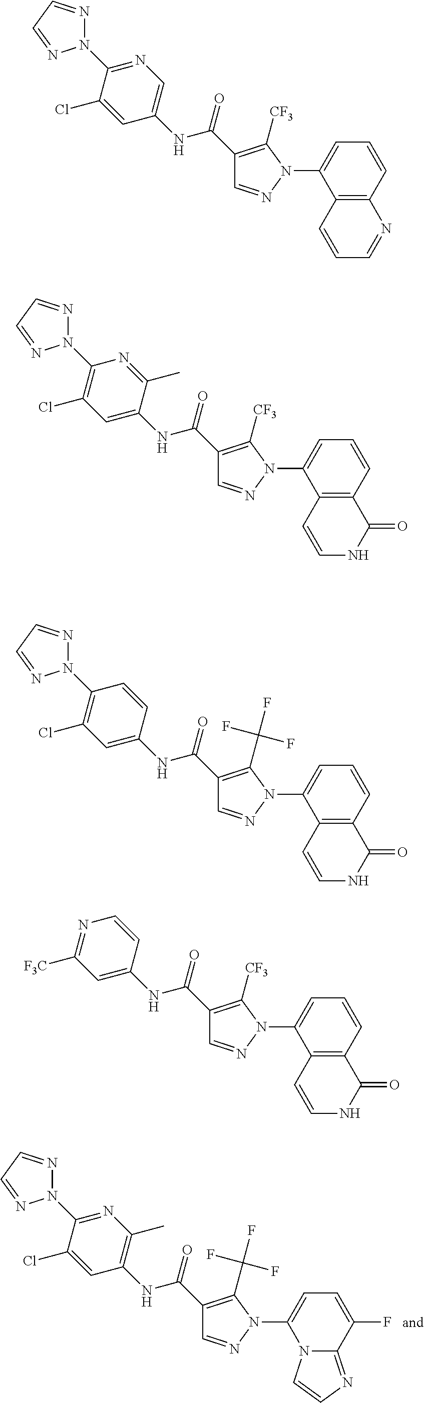 [SMILES: O=C(Nc1cnc(-n2nccn2)c(Cl)c1)c1cnn(-c2cccc3ncccc23)c1C(F)(F)F ; Cc1nc(-n2nccn2)c(Cl)cc1NC(=O)c1cnn(-c2cccc3c(=O)[nH]ccc23)c1C(F)(F)F ; O=C(Nc1ccc(-n2nccn2)c(Cl)c1)c1cnn(-c2cccc3c(=O)[nH]ccc23)c1C(F)(F)F ; O=C(Nc1ccnc(C(F)(F)F)c1)c1cnn(-c2cccc3c(=O)[nH]ccc23)c1C(F)(F)F ; Cc1nc(-n2nccn2)c(Cl)cc1NC(=O)c1cnn(-c2ccc(F)c3nccn23)c1C(F)(F)F ; "and"]

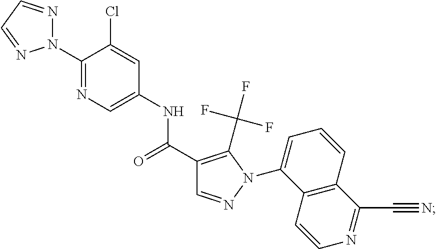 [SMILES: N#Cc1nccc2c(-n3ncc(C(=O)Nc4cnc(-n5nccn5)c(Cl)c4)c3C(F)(F)F)cccc12]

or an enantiomer, diastereomer, solvate or pharmaceutically acceptable salt form thereof. The compound may be a solvate. In particular, the compound may be a hydrate.

The API may be a compound of Formula (I), or an enantiomer, diastereomer or pharmaceutically acceptable salt form thereof, in amorphous state, dispersed state or dissolved state (i.e. molecular dispersion).

The compound of Formula (I) may be 1-(1-oxo-1,2-dihydroisoquinolin-5-yl)-5-(trifluoromethyl)-N-(2-(trifluoromethyl)pyridin-4-yl)-1H-pyrazole-4-carboxamide (Compound A). Compound A corresponds with the following structure:

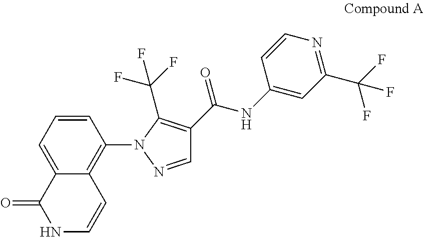 [SMILES: O=C(Nc1ccnc(C(F)(F)F)c1)c1cnn(-c2cccc3c(=O)[nH]ccc23)c1C(F)(F)F]

The API may be Compound A or a solvate or pharmaceutically acceptable salt form thereof. The API may be Compound A or a pharmaceutically acceptable salt form thereof. The API may be Compound A in a solvated form, for example as a hydrate (e.g. a monohydrate). In particular, the API is Compound A. In particular, the API is Compound A or a pharmaceutically acceptable salt form thereof in amorphous form, dispersed state or dissolved state. In particular, the API is Compound A in amorphous form, dispersed state or dissolved state.

In particular, the API used as starting material in the process to prepare a pharmaceutical formulation as described herein, is Compound A, or a solvated form or a pharmaceutically acceptable salt form thereof; while the API in the final pharmaceutical formulation or solid dosage form is Compound A or a pharmaceutically acceptable salt form thereof in amorphous form, dispersed state or dissolved state.

In particular, the API used as starting material in the process to prepare a pharmaceutical formulation as described herein, is Compound A in a solvated form, or a pharmaceutically acceptable salt form thereof; while the API in the final pharmaceutical formulation or solid dosage form is Compound A or a pharmaceutically acceptable salt form thereof in amorphous form, dispersed state, or dissolved state (i.e. molecular dispersion).

In particular, the API used as starting material in the process to prepare a pharmaceutical formulation as described herein, is Compound A hydrate (e.g. monohydrate) or a pharmaceutically acceptable salt form thereof, while the API in the final pharmaceutical formulation or solid dosage form is Compound A or a pharmaceutically acceptable salt form thereof in amorphous form, dispersed state, or dissolved state.

In particular, the API used as starting material in the process to prepare a pharmaceutical formulation as described herein, is Compound A hydrate (e.g. monohydrate); while the API in the final pharmaceutical formulation or solid dosage form is Compound A.

In particular, the API used as starting material in the process to prepare a pharmaceutical formulation as described herein, is Compound A hydrate (e.g. monohydrate); while the API in the final pharmaceutical formulation or solid dosage form is Compound A in amorphous form, dispersed state, or dissolved state.

Compounds of formula (I) can be synthesised according to the procedures disclosed in WO 2018/119036, which is incorporated herein by reference in its entirety.

It will be appreciated that any of the above discussion relating to active pharmaceutical ingredients may apply to any embodiment of the pharmaceutical formulations, solid dosage forms, processes and treatments described herein.

In a particular embodiment, the API in the pharmaceutical formulation as described herein is Compound A, or a pharmaceutically acceptable salt form thereof. In a particular embodiment, the API in the pharmaceutical formulation as described herein is Compound A.

In a particular embodiment, the API in the pharmaceutical formulation as described herein is a compound of formula (I) or an enantiomer, diastereomer, solvate, or pharmaceutically acceptable salt form thereof in amorphous form, dispersed state, or dissolved state. In a particular embodiment, the API in the pharmaceutical formulation as described herein is Compound A or a pharmaceutically acceptable salt form thereof, in amorphous form, dispersed state, or dissolved state. In a particular embodiment, the API in the pharmaceutical formulation as described herein is Compound A in amorphous form, dispersed state, or dissolved state.

In an embodiment, the API is soluble in the molten second component. In an embodiment, the API is soluble in the second component molten at 5° C. above the drop point of said second component. The API may have a solubility of at least about 1, 5, 10, 20, 50, 100, 200, 250, 300, 350 or 400 mg/mL in the second component at a temperature of 60° C. The API may have a solubility ranging from 1-400 mg/mL in the second component at a temperature of 60° C. The API may have a solubility ranging from 1-350 mg/mL in the second component at a temperature of 60° C., in particular ranging from 1-300 mg/mL in the second component at a temperature of 60° C., more in particular ranging from 1-250 mg/mL in the second component at a temperature of 60° C. The API may have a solubility ranging from 20-400 mg/mL in the second component at a temperature of 60° C. The API may have a solubility ranging from 20-350 mg/mL in the second component at a temperature of 60° C., in particular ranging from 20-300 mg/mL in the second component at a temperature of 60° C., more in particular ranging from 20-250 mg/mL in the second component at a temperature of 60° C. The API may have a solubility ranging from 100-400 mg/mL in the second component at a temperature of 60° C. The API may have a solubility ranging from 100-350 mg/mL in the second component at a temperature of 60° C., in particular ranging from 100-300 mg/mL in the second component at a temperature of 60° C., more in particular ranging from 100-250 mg/mL in the second component at a temperature of 60° C.

In particular, the API is sufficiently soluble in the molten second component to enable a therapeutically effective dose of the API to be administered in a formulation of the invention. In particular, the solubility of the API in the formulation is sufficient to ensure long term physical stability in a dissolved state at the desired concentration in the formulation. The concentration of API may be as high as deemed necessary to limit the size of the particular dosage form (e.g. capsule size and number) to be taken by a patient in order to reach the therapeutically effective dose. For example, if a capsule size of at most size 00 (dosage form volume=1 mL) is recommended to allow ease of swallowability and if the estimated targeted therapeutic dose is up to 1 g, 5 capsules of a 200 mg/dosage form formulation per day would be desired for a patient to reach the therapeutically effective targeted dose. Therefore, in this example, the API would have a solubility (at 60° C.) of at least 200 mg/mL in the formulation, preferably at least 220 mg/mL to account for incomplete filling of a 1 mL capsule. Lower solubility would represent an increase in the number of capsules in order to reach the estimated therapeutically effective dose.

Solubility may be measured using a classical shake-flask determination (within a range using visual assessment). This method is typically used for determination of solubility at a temperature above the drop point of the second component.

Solubility may be measured using hot stage microscopy or differential scanning microscopy (DSC). This method is typically used for determination of solubility at room temperature.

In an embodiment, the API forms a dispersion in the molten second component. The API may be fully solubilised in the molten second component. The API may form a suspension in the molten second component. The API may be partially in solution and partially as a suspension in the molten second component.

In an embodiment, the API has poor solubility in water. In an embodiment, the API has a solubility of at most about 50, 20, 10, 1, 0.1, 0.01, or 0.001 mg/mL in water. Solubility may be measured e.g. at 25° C. or 50° C. using the shake-flask method. The API may be defined as sparingly soluble (from 30 to 100 parts water for 1 part API), slightly soluble (from 100 to 1000 parts water for 1 part API), very slightly soluble (from 1000 to 10,000 parts water for 1 part API), or practically insoluble (more than 10,000 parts water for 1 part API) in water, as defined by The Pharmacopeia of the United States of America, in the chapter “General notices and Requirements” (Page information USP42-NF37 2S-9081; Section 5.30 Description and Solubility).

### Solid Dosage Form

The invention also provides a solid dosage form comprising a pharmaceutical formulation as described herein.

The solid dosage form may comprise a capsule encapsulating the pharmaceutical formulation. The capsule may be a hard capsule. The hard capsule may be a gelatin capsule (e.g. ConiSnap®, Licaps®, or Quali-G™) or a hydroxypropyl methylcellulose (HPMC) capsule (e.g. Vegicap®, VCaps®, VCaps® Plus, or Quali-V®). The hard capsule encapsulates a unit dose of the formulation. In embodiments where the formulation comprises fatty acid and polyethylene glycol monoesters and diesters and is substantially free of fatty acid and glycerol monoesters, diesters and triesters, the solid dosage form may preferably comprise an HPMC capsule. In embodiments where the formulation comprises fatty acid and polyethylene glycol monoesters and diesters and fatty acid and glycerol monoesters, diesters and triesters, the solid dosage form may preferably comprise a hard gelatin capsule. The capsule may be a soft capsule (e.g. a soft gelatin capsule).

The dosage form may be an oral dosage form (e.g. a capsule for oral administration). Alternatively, the dosage form may be an enteral dosage form.

Typically a hard capsule (e.g. a hard gelatin or HPMC capsule) comprises two part capsule shells, one of which is first filled with the formulation, the other of which is connected to the first in a telescoping manner to close the capsule. The two part capsule shells are typically adhered together by applying solvent (e.g. water or hydroalcohol, e.g. aqueous ethanol) to the interface between the two shells to create a bond between the two part shells. Alternatively, the two part shells may be sealed by applying a liquid banding agent (e.g. a liquid gelatin solution or a liquid HPMC solution), which solidifies to form a water-tight seal.

This differs to the manufacturing processes used for soft gelatin capsules, wherein the formulation is enclosed between half-capsule shells as the soft capsule is formed.

Hard gelatin (hard gel) or HPMC capsules are generally used for solid, semi-solid, and some compatible liquid formulations, while soft gelatin (soft gel) capsules are generally used for liquid formulations. Hard gel or HPMC capsules may be preferable for some formulations. Soft gel capsules contain a higher percentage of water than hard gel or HPMC capsules. This can result in problems when the soft gel contains liquid formulations of poorly water soluble APIs. Water leaching from the soft gel capsule into the formulation may lower the maximum drug loading for that capsule. Higher maximum drug load may be achieved for a poorly water soluble drug when using a hard gel or HPMC capsule compared to a soft gel capsule.

Additionally, hard gel or HPMC capsules can more easily be used in blister packs than soft gel capsules, as there is a lower risk of bursting the capsule when forcing it through the foil of the blister.

The solid dosage form may alternatively be a tablet.

The solid dosage form as described herein (e.g. a capsule, e.g. a hard gelatin or HPMC capsule) may contain about 0.1 mg to about 3000 mg of the API, or any particular amount or range therein, in particular from about 1 mg to about 1000 mg of the API, or any particular amount or range therein, or, more particularly, from about 10 mg to about 500 mg of the API, or any particular amount or range therein, of API in a regimen of about 1 to about (4×) per day for an average (70 kg) human; although, it is apparent to one skilled in the art that the therapeutically effective amount for said API will vary as will the diseases, syndromes, conditions, and disorders being treated.

The solid dosage form as described herein (e.g. a capsule, e.g. a hard gelatin or HPMC capsule) may contain about 2 to about 1000 mg of the API. In embodiments where the API is 1-(1-oxo-1,2-dihydroisoquinolin-5-yl)-5-(trifluoromethyl)-N-(2-(trifluoromethyl)pyridin-4-yl)-1H-pyrazole-4-carboxamide (Compound A), the solid dosage form may comprise about 2 to about 1000 mg, about 10 to about 200 mg, or about 50 to about 200 mg of Compound A. The solid dosage form may comprise 2, 10, 50, 100 or 200 mg of Compound A. The solid dosage form may comprise 50, 100, 150 or 200 mg of Compound A.

In embodiments where the API is 1-(1-oxo-1,2-dihydroisoquinolin-5-yl)-5-(trifluoromethyl)-N-(2-(trifluoromethyl)pyridin-4-yl)-1H-pyrazole-4-carboxamide (Compound A) or a pharmaceutically acceptable salt form thereof, the solid dosage form may comprise about 2 to about 1000 mg, about 10 to about 200 mg or about 50 to about 200 mg of Compound A or a pharmaceutically acceptable salt form thereof. The solid dosage form may comprise 2, 10, 50, 100, 150 or 200 mg of Compound A or a pharmaceutically acceptable salt form thereof. The solid dosage form may comprise 50, 100, 150 or 200 mg of Compound A or a pharmaceutically acceptable salt form thereof.

In a particular embodiment, the solid dosage form is a capsule comprising a first component and a second component, as described herein.

In a particular embodiment, the solid dosage form is a capsule comprising a first component and a second component, as described herein, and an antioxidant.

In a particular embodiment, the solid dosage form is a capsule comprising a first component and a second component, as described herein, an antioxidant, and a crystallisation rate inhibitor.

In a particular embodiment, the solid dosage form is a tablet comprising a first component and a second component, as described herein.

In a particular embodiment, the solid dosage form is a tablet comprising a first component and a second component, as described herein, and an antioxidant.

In a particular embodiment, the solid dosage form is a tablet comprising a first component and a second component, as described herein, an antioxidant, and a crystallisation rate inhibitor.

In a particular embodiment, the solid dosage form is a capsule consisting of a first component and a second component, as described herein.

In a particular embodiment, the solid dosage form is a capsule consisting of a first component and a second component, as described herein, and an antioxidant.

In a particular embodiment, the solid dosage form is a capsule consisting of a first component and a second component, as described herein, an antioxidant, and a crystallisation rate inhibitor.

In a particular embodiment, the solid dosage form is a tablet consisting of a first component and a second component, as described herein.

In a particular embodiment, the solid dosage form is a tablet consisting of a first component and a second component, as described herein, and an antioxidant.

In a particular embodiment, the solid dosage form is a tablet consisting of a first component and a second component, as described herein, an antioxidant, and a crystallisation rate inhibitor.

In a particular embodiment, the solid dosage form is a capsule comprising a pharmaceutical formulation of the present invention. In a particular embodiment, the solid dosage form is a tablet comprising a pharmaceutical formulation of the present invention.

In an embodiment, the solid dosage form comprises a pharmaceutical formulation, wherein the formulation comprises 50, 100, 150 or 200 mg of 1-(1-oxo-1,2-dihydroisoquinolin-5-yl)-5-(trifluoromethyl)-N-(2-(trifluoromethyl)pyridin-4-yl)-1H-pyrazole-4-carboxamide:

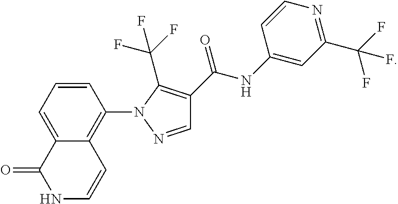 [SMILES: O=C(Nc1ccnc(C(F)(F)F)c1)c1cnn(-c2cccc3c(=O)[nH]ccc23)c1C(F)(F)F]

In an embodiment, the solid dosage form comprises a pharmaceutical formulation, wherein the formulation comprises 50, 100, 150 or 200 mg of 1-(1-oxo-1,2-dihydroisoquinolin-5-yl)-5-(trifluoromethyl)-N-(2-(trifluoromethyl)pyridin-4-yl)-1H-pyrazole-4-carboxamide, or a solvate or pharmaceutically acceptable salt form thereof, calculated based on the free base form.

The capsule of the solid dosage form may have the role of the crystallisation rate inhibitor. For example, the capsule might be an HPMC capsule.

The crystallisation rate inhibitor might be part of the solid dosage form in tablet form. For example, an HPMC tablet.

The invention also relates to a solid dosage form comprising a first component and a second component, as described herein; wherein the solid dosage form is a capsule acting as crystallisation rate inhibitor, e.g. a HPMC capsule.

The invention also relates to a solid dosage form consisting of a first component and a second component, as described herein; wherein the solid dosage form is a capsule acting as crystallisation rate inhibitor, e.g. a HPMC capsule.

The invention also relates to a solid dosage form comprising a first component and a second component, as described herein; wherein the solid dosage form is in tablet form and wherein the crystallisation rate inhibitor is part of the tablet, e.g. a HPMC tablet.

The invention also relates to a solid dosage form consisting of a first component and a second component, as described herein; wherein the solid dosage form is in tablet form and wherein the crystallisation rate inhibitor is part of the tablet, e.g. a HPMC tablet.

For oral administration, a solid dosage form is in particular provided in the form of tablets containing about 1.0, about 10, about 50, about 100, about 150, about 200, about 250, and about 500 milligrams of API; in particular from about 25 mg to about 500 mg of API.

For oral administration, a solid dosage form is in particular provided in the form of capsules containing about 1.0, about 2, about 10, about 50, about 100, about 150, about 200, about 250, and about 500 milligrams of API; in particular from about 25 mg to about 500 mg of API.

Advantageously, the API may be administered in a single daily dose, or the total daily dosage may be administered in divided doses of two, three and 4×daily.

Optimal dosages of the pharmaceutical formulation to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

The invention also provides a process for preparing a solid or semi-solid pharmaceutical formulation, as described herein. The process may comprise the steps of
a) forming a melt comprising a first component and a second component, as described herein, wherein the forming a melt step comprises heating the second component; and
b) cooling the melt;
   to provide a solid or semi-solid pharmaceutical formulation as described herein.

The invention also provides a process for preparing a solid dosage form, as described herein. The process may comprise the steps of:
a) forming a melt comprising a first component and a second component, as described herein, wherein the forming a melt step comprises heating the second component;
b) filling a capsule with the melt; and
c) cooling the filled capsule;
   to provide a solid dosage form as described herein.

In an embodiment, the melt is formed under an inert atmosphere. In another embodiment, the melt is formed under nitrogen.

In an embodiment, the melt further comprises an antioxidant, for example all-rac-alpha-tocopherol. The melt may further comprise a crystallisation rate inhibitor, for example HPMC or PVPVA. The melt may further comprise one or more pharmaceutically acceptable excipients, as described herein.

The step of forming a melt may comprise heating the second component to a temperature above its drop point and the cooling step may be performed by cooling to below the drop point of the second component. The second component may be heated to a temperature of at least about 5, 10, or 15° C. above its drop point. In particular the second component may be heated to a temperature of at least 5, 10, or 15° C. above the upper limit of its drop point. The second component may be heated to a temperature of at least about 5° C. above its drop point. The second component may be heated to a temperature of at least about 10° C. above its drop point. The second component may be heated to a temperature of at most about 20° C. above its drop point. The second component may be heated to a temperature of up to about 70° C., for example from about 50° C. to about 70° C. The second component may be heated to a temperature of about 60° C. The cooling step may comprise cooling the melt to room temperature (e.g. 25° C.).

The step of forming a melt may comprise adding the API (and, optionally, a crystallisation rate inhibitor and/or antioxidant, where present) to the molten second component. The step of forming a melt may comprise mixing the second component and API, (and, optionally, a crystallisation rate inhibitor and/or antioxidant, where present) and then melting the resulting mixture. Where an antioxidant is present in the formulation, the step of forming a melt may comprise mixing the second component and the antioxidant (and, optionally, a crystallisation rate inhibitor, if present in the formulation), melting the resulting mixture, and then adding the API (and, optionally, a crystallisation rate inhibitor, if present in the formulation) to the molten mixture. In these embodiments, the forming a melt step may comprise heating the second component to a temperature above its drop point.

In particular, the melt is a semi-liquid melt or liquid melt. In particular, the melt is a liquid melt.

The API used as starting material in the process to prepare the pharmaceutical formulation according to the present invention, wherein the API is Compound A, or a solvate or pharmaceutically acceptable salt thereof, may be a crystalline form of Compound A monohydrate, in particular a crystalline form of Compound A monohydrate producing an X-ray powder diffraction pattern comprising peaks at 16.4, 23.7 and 25.7 degrees two theta±0.2 degrees two theta. The X-ray powder diffraction pattern may further comprise peaks at 13.6, 17.9, 22.6, 24.5, 25.2 and 27.1 degrees two theta ±0.2 degrees two theta. The X-ray powder diffraction pattern may further comprise at least one peak selected from 8.3, 8.6, 11.5, 14.0, 15.4, 17.5, 19.7, 22.0, 22.2, 24.0 and 29.9 degrees two theta±0.2 degrees two theta. The X-ray powder diffraction pattern may comprise peaks at 8.3, 8.6, 11.5, 13.6, 14.0, 15.4, 16.4, 17.5, 17.9, 19.7, 22.6, 23.7, 24.5, 25.2, 25.7, and 27.1 degrees two theta±0.2 degrees two theta. The X-ray powder diffraction pattern may comprise peaks at 11.5, 16.4, 19.7, 23.7 and 25.7 degrees two theta±0.2 degrees two theta.

The API used as starting material in the process to prepare the pharmaceutical formulation according to the present invention, wherein the API is Compound A, or a solvate or pharmaceutically acceptable salt thereof, may be a crystalline form of Compound A hydrate, in particular a crystalline form of Compound A hydrate producing an X-ray powder diffraction pattern comprising peaks at 8.4, 12.7, 13.3 and 16.7 degrees two theta±0.2 degrees two theta. The X-ray powder diffraction pattern may further comprise at least one peak selected from 6.7, 10.0, 10.7, 12.0, 12.3, 13.5, 14.1, 14.6, 15.4, 15.6, 16.0, 18.1, 18.4, 19.2, 20.0, 20.3, 21.1, 22.0 and 24.9 degrees two theta ±0.2 degrees two theta.

In an embodiment of the process for preparing a solid dosage form, the capsule is a hard capsule (e.g. a hard gel capsule or an HPMC capsule). The hard capsule may be filled using a hard capsule filling machine hopper. The machine hopper may be preheated to a temperature above the drop point of the second component, wherein the temperature is as described above.

The filled capsule may be cooled to a temperature below the drop point of the second component so that the pharmaceutical formulation solidifies. The capsule may be stored at room temperature (e.g. 25° C.) following the filling step, to ensure the formulation solidifies. Additionally, the hard capsules may be sealed or banded. This may protect the contents of the capsule from leakage and/or improve the stability of the formulation during storage or during use. A hard capsule may be sealed by adhering the two part capsule shells together by applying solvent (e.g. water or hydroalcohol, e.g. aqueous ethanol) to the interface between the two shells to create a bond between the two part shells. Alternatively, the two part shells may be sealed by applying a liquid banding agent (e.g. a liquid gelatin solution or a liquid HPMC solution), which solidifies to form a water-tight seal.

In an embodiment of the process for preparing a solid dosage form, the capsule is a soft capsule (e.g. a soft gel capsule). The process may comprise the step of forming the soft gel capsule, prior to filing the capsule with the melt. This step may be carried out using a soft capsule filing machine. The filing machine may be preheated to a temperature above the drop point of the second component, wherein the temperature is as described above. The filled capsule may be cooled to a temperature below the drop point of the second component so that the pharmaceutical formulation solidifies. The capsule may be stored at room temperature (e.g. 25° C.) following the filling step, to ensure the formulation solidifies.

The process may further comprise the step of packaging the capsules in bottles (e.g. HDPE bottles), followed by induction sealing of the bottles. Alternatively, the process may further comprise the step of packaging the capsules into blister packs and sealing the blister packs.

This process may be advantageous compared to traditional processes for manufacturing solid dosage forms. The molten formulation can be easily dispensed into a capsule and then allowed to solidify. This reduces the number of steps usually associated with the manufacture of solid (or semi-solid) formulations.

A solid dosage form of the invention may be prepared using a spray congealing process, comprising the steps of: a) forming a melt comprising the first component and the second component, as described herein (and, optionally, an antioxidant and/or a crystallisation rate inhibitor); and b) atomizing the melt into cold nitrogen. The atomised melt may be compressed into tablets.

A solid dosage form of the invention may be prepared by a screw granulation process, for example using twin-screw extruders that continuously mix and granulate the first component and the second component, as described herein (and, optionally, an antioxidant and/or a crystallisation rate inhibitor), and optionally maltodextrin. The resulting granules may be compressed into tablets.

A solid dosage form of the invention may be prepared by loading a melt of the first component and the second component, as described herein (and, optionally, an antioxidant and/or a crystallisation rate inhibitor) onto a porous clay-type particle, such as magnesium aluminometasilicate (e.g. Neusilin®) or silica, to obtain a powder which may be compressed into tablets.

It will be appreciated that any of the above discussion relating to solid dosage forms and processes for their preparation may apply to any embodiments of solid dosage forms, processes and treatments described herein.

### Methods of Treatment

The pharmaceutical formulations described herein may be administered in any of the foregoing dosage forms and regimens or by means of those dosage forms and regimens established in the art whenever use of the pharmaceutical formulation is required for a subject in need thereof.

The pharmaceutical formulations and dosage forms of the present invention are useful in methods for treating, ameliorating and/or preventing a disease, a syndrome, a condition or a disorder that is affected by the inhibition of MALT1 in a subject in need thereof. Such methods comprise, consist of and/or consist essentially of administering to a subject, including an animal, a mammal, and a human in need of such treatment, amelioration and/or prevention, a therapeutically effective amount of a formulation or dosage form described herein.

One embodiment of the present invention is directed to a method of treating a MALT1-dependent or MALT1-mediated disease or condition in a subject in need thereof, including an animal, a mammal, and a human in need of such treatment, comprising administering to the subject a therapeutically effective amount of a pharmaceutical formulation or dosage form described herein.

In another embodiment, the MALT1-dependent or MALT1-mediated disease or condition is selected from cancers of hematopoietic origin or solid tumors such as chronic myelogenous leukemia, myeloid leukemia, non-Hodgkin lymphoma, and other B cell lymphomas.

In particular, pharmaceutical formulations and dosage forms of the invention are useful for treating or ameliorating diseases, syndromes, conditions, or disorders such as diffuse large B-cell lymphoma (DLBCL), mantle cell lymphoma (MCL), follicular lymphoma (FL), and mucosa-associated lymphoid tissue (MALT) lymphoma.

More particularly, pharmaceutical formulations and dosage forms of the invention are useful for treating or ameliorating diffuse large B-cell lymphoma (DLBCL), mantle cell lymphoma (MCL), follicular lymphoma (FL), and mucosa-associated lymphoid tissue (MALT) lymphoma, comprising administering to a subject in need thereof a therapeutically effective amount of a pharmaceutical formulation or dosage form described herein.

Further, pharmaceutical formulations and dosage forms described herein are useful for treating or ameliorating an immunological disease, syndrome, disorder, or condition selected from the group consisting of rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD).

In an embodiment, cancers that may benefit from a treatment with pharmaceutical formulations and dosage forms described herein include, but are not limited to, lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

In another embodiment, pharmaceutical formulations and dosage forms of the invention may be used for the treatment of immunological diseases including, but not limited to, autoimmune and inflammatory disorders, e.g. arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

One embodiment of the present invention is directed to a method of treating a disease, syndrome, condition, or disorder, wherein said disease, syndrome, condition, or disorder is affected by the inhibition of MALT1, comprising administering to a subject in need thereof a therapeutically effective amount of a pharmaceutical formulation or dosage form described herein.

In a further embodiment, the disease, syndrome, condition, or disorder is selected from the group consisting of diffuse large B-cell lymphoma (DLBCL), mantle cell lymphoma (MCL), follicular lymphoma (FL), and mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD).

In a further embodiment, the disease, syndrome, condition, or disorder is selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia.

In one embodiment, the present invention is directed to a method of treating a disease, syndrome, condition, or disorder selected from the group consisting of diffuse large B-cell lymphoma (DLBCL), mantle cell lymphoma (MCL), follicular lymphoma (FL), and mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD), comprising administering to a subject in need thereof a therapeutically effective amount of a pharmaceutical formulation or dosage form described herein.

In another embodiment, the present invention is directed to a method of treating a disease, syndrome, condition, or disorder selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia, comprising administering to a subject in need thereof a therapeutically effective amount of a pharmaceutical formulation or dosage form described herein. In a further embodiment, the disease, syndrome, condition, or disorder is non-Hodgkin's lymphoma (NHL). In a further embodiment, the non-Hodgkin's lymphoma (NHL) is B-cell NHL.

In another embodiment, the present invention is directed to a pharmaceutical formulation described herein for the preparation of a medicament for treating a disease, syndrome, disorder or condition selected from the group consisting of diffuse large B-cell lymphoma (DLBCL), mantle cell lymphoma (MCL), follicular lymphoma (FL), and mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD), in a subject in need thereof.

In another embodiment, the present invention is directed to a pharmaceutical formulation described herein for the preparation of a medicament for treating a disease, syndrome, condition, or disorder selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia, in a subject in need thereof. In a further embodiment, the disease, syndrome, condition, or disorder is non-Hodgkin's lymphoma (NHL). In a further embodiment, the non-Hodgkin's lymphoma (NHL) is B-cell NHL.

In another embodiment, a pharmaceutical formulation or dosage form described herein is for use in a method for treating a disorder selected from the group consisting of diffuse large B-cell lymphoma (DLBCL), mantle cell lymphoma (MCL), follicular lymphoma (FL), and mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD), in a subject in need thereof.

In another embodiment, a pharmaceutical formulation or dosage form described herein is for use in a method for treating a disease, syndrome, condition, or disorder selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia, in a subject in need thereof. In a further embodiment, the disease, syndrome, condition, or disorder is non-Hodgkin's lymphoma (NHL), in a subject in need thereof. In a further embodiment, the non-Hodgkin's lymphoma (NHL) is B-cell NHL.

In another embodiment of the present invention, the pharmaceutical formulations described herein may be employed in combination with one or more other medicinal agents, more particularly with other anti-cancer agents, e.g. chemotherapeutic, anti-proliferative or immunomodulating agents, or with adjuvants in cancer therapy, e.g. immunosuppressive or anti-inflammatory agents.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All possible combinations of the above-indicated embodiments are considered to be embraced within the scope of this invention.

Reference is now made to the following examples, which illustrate the invention in a non-limiting fashion.

### General Synthetic Methods

Representative compounds of the present invention can be synthesized in accordance with the general synthetic methods described below and illustrated in the schemes and examples that follow. Since the schemes are an illustration, the invention should not be construed as being limited by the chemical reactions and conditions described in the schemes and examples. Compounds analogous to the target compounds of these examples can be made according to similar routes. The disclosed compounds are useful as pharmaceutical agents as described herein. The various starting materials used in the schemes and examples are commercially available or may be prepared by methods well within the skill of persons versed in the art.

Abbreviations used in the instant specification, particularly the schemes and examples, are as follows:
Ac₂O acetic anhydride
AcOH acetic acid
API active pharmaceutical ingredient
BOP (benzotriazol-1-yloxy)tris(dimethylamino)phosphonium hexafluorophosphate
BPO benzoyl peroxide
Bu butyl
cat. catalyst
DBU 1,8-diazabicyclo[5.4.0]undec-7-ene
DCM dichloromethane
DMA dimethylacetamide
DIPEA N,N-diisoproylethylamine
DMF dimethylformamide
DMSO dimethyl sulfoxide
DSC differential scanning calorimetry
Et ethyl
EtOH ethyl alcohol
FaSSIF fasted-state simulated intestinal fluid
h hour(s)
HATU O-(7-azabenzotriazol-1-yl)-N,N,N′,N′-tetramethyluronium hexafluorophosphate
HDPE high-density polyethylene
HPLC high performance liquid chromatography
LDPE low-density polyethylene
LED light-emitting diode
m-CPBA meta-chloroperoxybenzoic acid
Me methyl
MeOH methyl alcohol
mg milligram
min minute
NH₄Cl ammonium chloride
NMP N-methyl-2-pyrrolidone
Pd(dppf)Cl₂ [1,1′-bis(diphenylphosphino)ferrocene]dichloropalladium
Pd(PPh₃)₄ tetrakis(triphenylphosphine)palladium
PK pharmacokinetic
PPh₃ triphenyl phosphine
Pt/C platinum on charcoal
PVPVA polyvinylpyrrolidone-vinyl acetate copolymer
TMPMgCl LiCl 2,2,6,6-tetramethylpiperidinylmagnesium chloride lithium chloride complex
TsOH toluenesulfonic acid
rpm revolutions per minute
rt or RT room temperature
TBAF tetrabutyl ammonium fluoride
TEA triethylamine
TMSI iodotrimethylsilane
t-Bu tert-butyl
TFA trifluoroacetic acid
TFAA trifluoroacetic anhydride
THE tetrahydrofuran
Xantphos 4,5-Bis(diphenylphosphino)-9,9-dimethylxanthene
XRPD X-ray powder diffraction

Compounds of Formula (Ia) wherein R₇ is hydrogen, may be prepared according to the process outlined in Scheme 1.

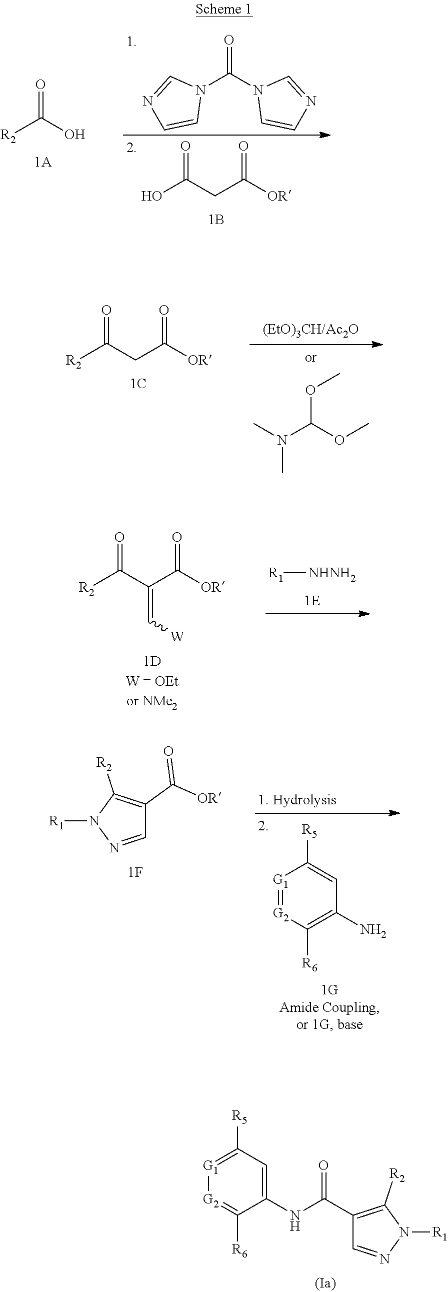

A carboxylic acid of formula (1A) may be treated with carbonyldiimidazole followed by addition of a mono-ester of malonic acid of formula (1B), wherein R′ is C₁₋₄alkyl, and a base, such as isopropylmagesium chloride, to yield a ketoester of formula (1C). Condensation with triethyl orthoformate in acetic anhydride or with 1,1-dimethoxy-N,N-dimethylmethanamine may yield a 2-ethoxymethylidene-3-oxo ester (or 2-(dimethylamino)methylidene-3-oxo ester) of formula (1D). A compound of formula (1D) may be reacted with a hydrazine of formula (1E) to provide a pyrazole of formula (1F). Hydrolysis of the ester group may be effected via by treatment with aqueous sodium hydroxide in the presence of an alcohol co-solvent, to provide the corresponding carboxylic acid intermediate, which, subsequently, may be converted to a compound of Formula (I) upon amide coupling with a compound of formula (1G). The amide coupling may be carried out, for example, in the presence of phosphorus oxychloride in pyridine to afford the corresponding acid chloride, followed by treatment with a compound of formula (1G), in the presence of a base. In one embodiment, the amide coupling reaction is carried out in the presence of a suitable amide coupling reagent such as HATU, in the presence of a base such as, but not limited to, diisopropylethyl amine.

Alternatively, the pyrazole ester of formula (1F) may be directly converted to a compound of Formula (I) via treatment with a compound of formula (1G) and a base, such as potassium tert-butoxide.

An alternate route to compounds of Formula (Ia) wherein R₇ is hydrogen, is illustrated in Scheme 2.

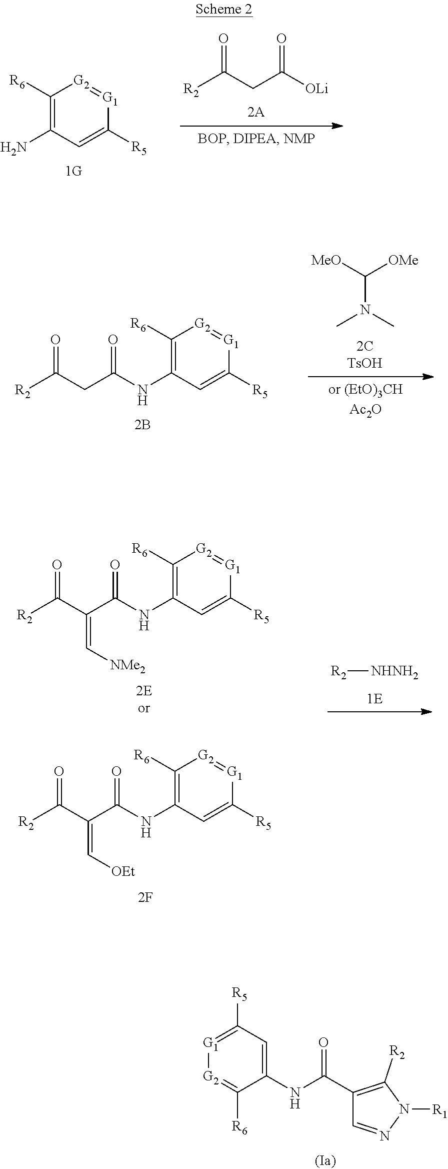

Aniline (1G) may be coupled with a lithium acetoacetate of formula (2A) in the presence of coupling reagent such as BOP, a base such as DIPEA, and a solvent such as NMP, to provide a compound of formula (2B). A compound of formula (2B) may then be reacted with DMF-DMA (2C) in the presence of an acid, such as TsOH, or reacted with triethoxymethane (2D) in AcOH to afford a compound of formula (2E) or (2F), respectively. A compound of formula (2E) or (2F) may then be treated with a hydrazine of formula (1E) to afford a compound of Formula (I).

Scheme 3 illustrates the preparation of certain hydrazine intermediates of formula (1E), useful for the preparation of compounds of Formula (I).

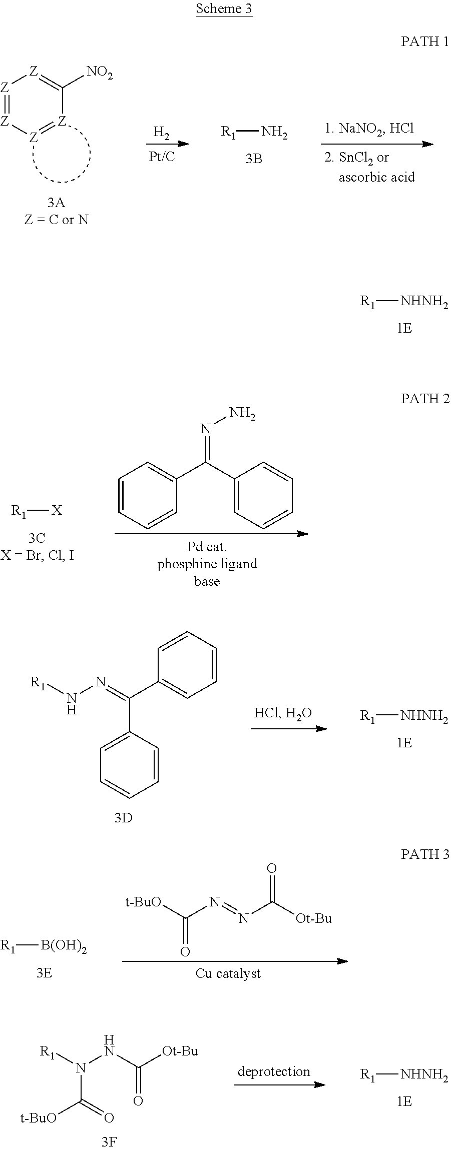

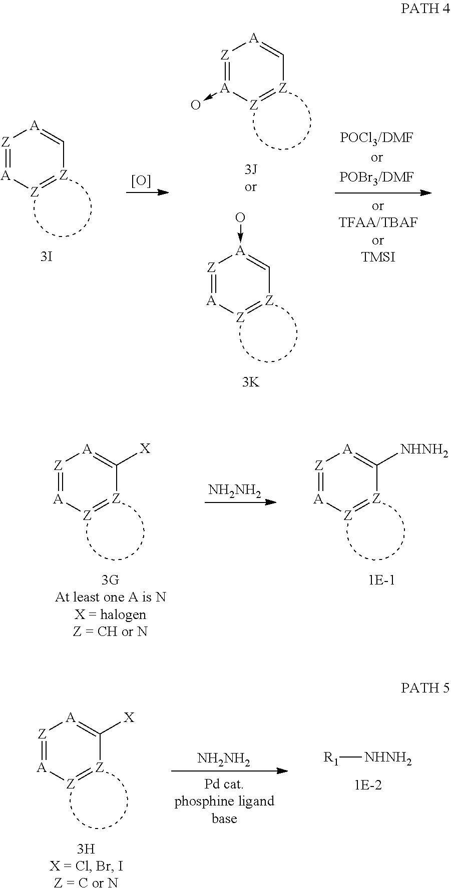

A heteroaryl amine of formula (3B) may be converted to a heteroaryl diazonium salt via treatment with sodium nitrite under acidic conditions. This intermediate may be reduced, using a reductant such as tin (II) chloride or ascorbic acid, to form the hydrazine of formula (1E). For heteroaryl amines of formula (3B) that are not commercially available, they may be accessed by reduction of the heteronitroarene (3A) using hydrogen and Pt/C or other conventional nitro-reducing conditions (path one).

R₁-substituted chlorides, bromides, and iodides may undergo a palladium catalyzed Buchwald Hartwig coupling with benzophenone hydrazine, in the presence of a ligand, such as Xantphos, and a base, such as sodium tert-butoxide, to form a hydrazine of formula (3D). Acidic hydrolysis may afford the hydrazine of formula (1E) (path two).

R₁-substituted boronic acids may also serve as a precursor to compounds of formula (1E) by the route shown in path three. A boronic acid of formula (3E) may undergo a Cu²⁺-catalyzed (such as Cu(OAc)₂, TEA in CH₂Cl₂) addition to di-tert-butylazodicarboxylate to afford an intermediate of formula (3F), which may be deprotected under acidic conditions to yield the compound of formula (1E). Heteroaryl hydrazines of formula (1E-1), having a nitrogen atom in the ortho- or para-position with respect to the hydrazine functionality, may be prepared via direct displacement of a halogen with hydrazine or hydrazine hydrate. (Hetero) haloarenes of formula (3G) that are not commercially available may be prepared from their corresponding (hetero)arenes (3I), with an oxidant such as mCPBA, to form the N-oxide (3J) (or (3K)) that may then be converted to (hetero) haloarene 3G via treatment with POCl₃ and DMF, POBr₃/DMF, TFAA/TBAF, or TMSI (path four). Alternatively, halogenated (hetero)arenes of formula (3H) may undergo palladium-catalyzed cross-coupling with hydrazine to directly furnish intermediate (1E-2) (path five).

Scheme 4 illustrates multiple pathways available for the synthesis of intermediate (1G-1), wherein G₁ is C(R₄).

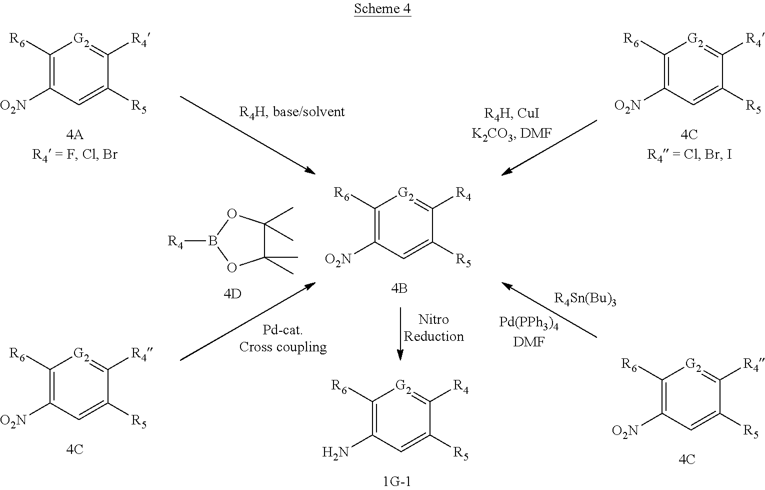

Compound (B-1) may be reacted with a compound of formula R₄H in the presence of a base, such as Cs₂CO₃, in a solvent, such as DMF, to yield a compound of formula (4B). Alternatively, a compound of formula (4C) may be treated with a crossing coupling reagent, such as a boron reagent of formula (4D) or a tin reagent of formula R₄Sn(Bu)₃; in the presence of a palladium catalyst, including but not limited to, Pd(dppf)Cl₂ or Pd(PPh₃)₄; in a suitable solvent or solvent system such as DMF, dioxane/water, or the like; to produce a compound of formula (4B). Another suitable pathway includes the reaction of a compound of formula (4C) with a compound of formula R₄H, in the presence of a coupling reagent such as CuI, with a base such as Cs₂CO₃, and in a solvent such as DMF, to afford a compound of formula (4B). A compound of formula (4B) may be reduced to a compound of formula (1G-1) using a reducing agent such as Zn or Fe in the presence of NH₄Cl, in a solvent such as MeOH.

Scheme 5 illustrates the preparation of certain compounds of Formula (I) wherein R₆ is other than hydrogen.

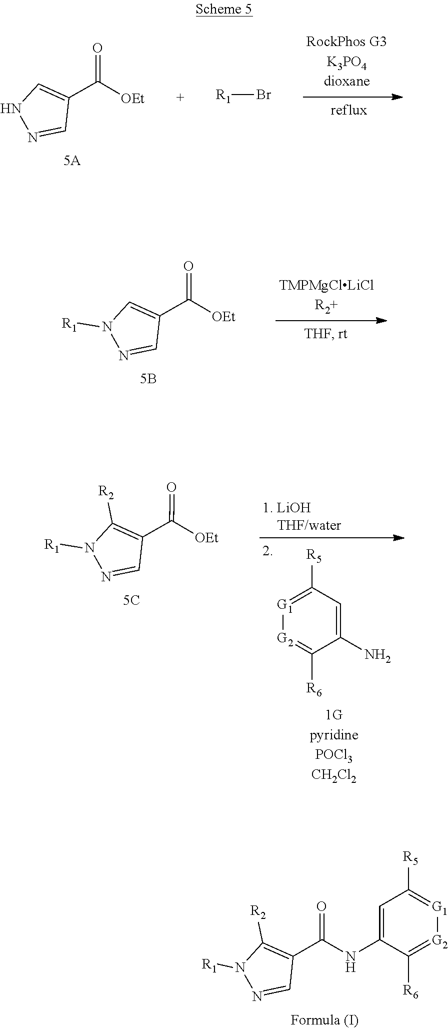

Scheme 6 illustrates the preparation of certain compounds of Formula (I).

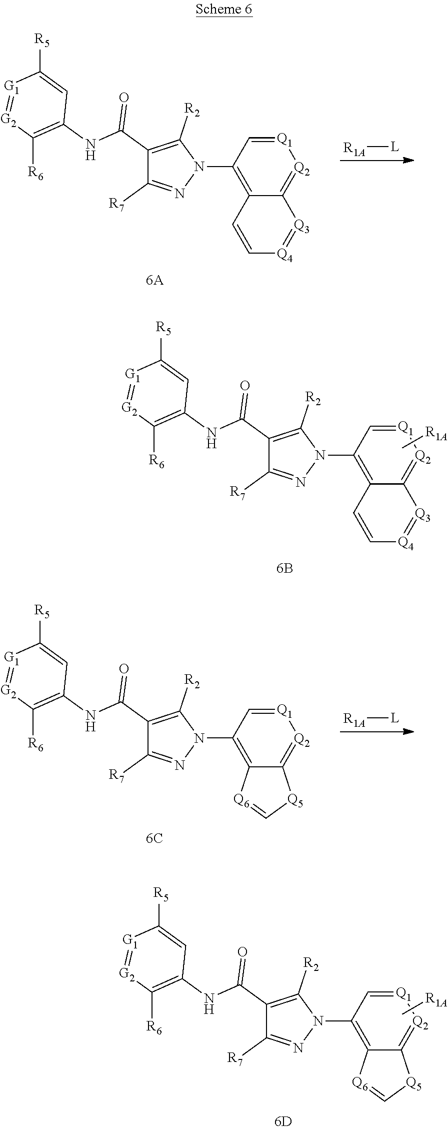

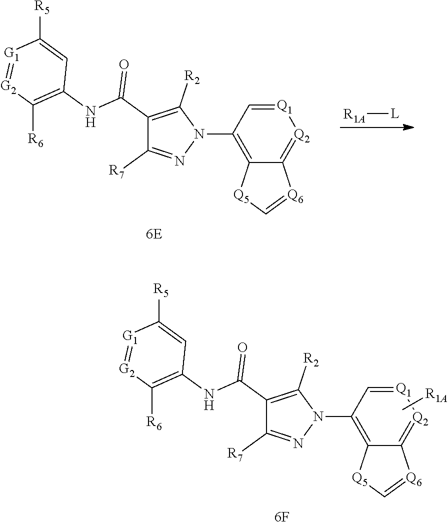

In the instance when L is H, alkylation of compounds of formulae 6A, 6C and 6E may occur via formation of a radical from R_{1A}-L, generated by treatment with ammonium persulfate and (IR[DF(CF₃)PPY]₂(DTBPY))PF₆, in a mixture of water and CH₃CN or DMSO and TFA, under irradiation with blue LED.

Alternatively, in the instance when L is H, alkylation of compounds of formulae 6A, 6C and 6E may occur via formation of a radical from R_{1A}-L, generated by treatment with BPO and (IR[DF(CF₃)PPY]₂(DTBPY))PF₆ in MEOH and TFA, under irradiation with blue LED.

When L is H, alkylation of compounds of formulae 6A, 6C and 6E may occur via formation of a radical from R_{1A}-L, generated by treatment with iron(II)sulfate heptahydrate and hydrogen peroxide, in a mixture of water and CH₃CN or DMSO and H₂SO₄.

When L is a zinc sulfonate, alkylation of compounds of formulae 6A, 6C and 6E may occur via formation of a radical from R_{1A}-L, generated by treatment with tert-butyl hydroperoxide, in a mixture of water and DCM and TFA.

Likewise, when L is —COOH or a BF₃-salt, alkylation of compounds of formulae 6A, 6C and 6E may occur via formation of a radical from R_{1A}-L, generated by treatment with ammonium persulfate and silver nitrate, in a mixture of water and DCM or CH₃CN or DMSO or dioxane and TFA.

Compounds of formulae 6A, 6C and 6E may also be converted to their corresponding N-oxides via treatment with an oxidizing agent such as m-CPBA in DCM or THF. Said N-oxides by optionally be converted to their corresponding ortho —CN derivatives using trimethylsilyl cyanide and DBU, in a solvent such as THF. Said N-oxides may also be converted to their alkoxy or cycloalkoxy derivatives by the action of tosylanhydride, Na₂CO₃ and an appropriately substituted alkyl-OH or cycloalkyl-OH reagent.

Alternatively, the N-oxides of compounds of formulae 6A, 6C and 6E may be converted to their corresponding ortho-chloro derivatives by the action of POCl₃, optionally in a solvent such as CHCl₃, which may be used as an intermediate for the preparation of C₁₋₆alkylthio, C₁₋₆cycloalkylthio, and sulfur-linked heterocyclic rings of the present invention. Similarly, the ortho-chloro derivatives may be reacted with appropriately substituted amines to afford C₁₋₆alkylamino, C₁₋₆cycloalkylamino, or N-linked heterocyclic rings of the present invention. Or, the ortho-chloro derivatives may undergo a Suzuki-type reaction in a subsequent step, with an appropriately substituted corresponding alkyl- or cycloalkyl-boronic acid to form a compound of Formula (I).

Compounds of Formula (I) can be synthesised according to the procedures disclosed in WO 2018/119036, which is incorporated herein by reference in its entirety.

### EXAMPLES

### XRPD Method

X-ray powder diffraction (XRPD) analysis was carried out on a Bruker (D8 Advance) X-ray powder diffractometer. The compound was spread on a mono-crystalline silicon plate and pressed gently to be flat and homogeneous for testing.

Samples were run on XRPD using the method below:
Tube: Cu: K-Alpha (λ=1.54056\acute{Å})
Generator: Voltage: 40 kV; Current: 40 mA
Detector: PSD: LynxEye
Divergence Slit: 0.60 mm; Primary Soller Slit: 2.5 deg.
Detector Slit: 10.50 mm; Antiscatter Slit: 7.10 mm
Sec. Soller Slit: 2.5 deg.
Scan type: Locked Coupled
Scan mode: Continuous Scan
Scan parameter: Scan axis: 2-Theta/Theta
Scan Scope: 3 to 50 deg.; Step size: 0.02 deg.
Time/step: 0.12 s
Sample rotation: 60 rpm
Scanning rate: 10 deg/min

One skilled in the art will recognize that diffraction patterns and peak positions are typically substantially independent of the diffractometer used and whether a specific calibration method is utilized. Typically, the peak positions may differ by about +0.2° two theta, or less. The intensities (and relative intensities) of each specific diffraction peak may also vary as a function of various factors, including, but not limited to particle size, orientation, sample purity, etc.

### Example 1: Preparation of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A) Hydrate

Compound A hydrate was prepared by analogy to the synthesis method as described in Example 158 of WO 2018/119036. The compound prepared by this method was confirmed to be a hydrate crystalline form.

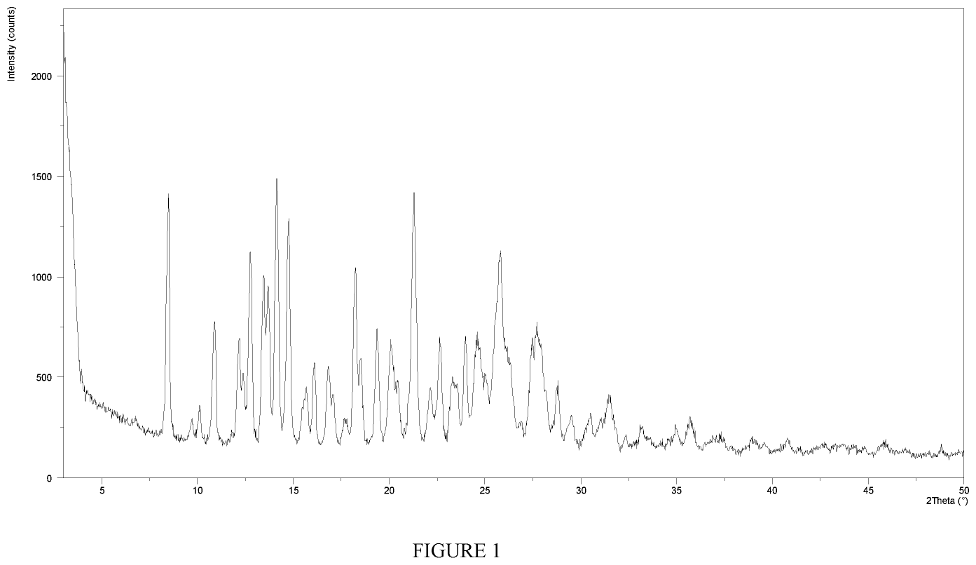

The crystalline hydrate was characterized by XRPD (see FIG. 1). Table 1 provides peak listings and relative intensities for the XPRD.

TABLE 1

| Pos. [°2Th.] | Rel. Int. [%] |
|---|---|
| 3.3492 | 39.37 |
| 6.6640 | 4.90 |
| 8.3921 | 99.18 |
| 9.5561 | 2.00 |
| 9.9822 | 17.19 |
| 10.4253 | 1.40 |
| 10.7270 | 21.94 |
| 12.0003 | 10.48 |
| 12.2582 | 8.63 |
| 12.6973 | 75.08 |
| 13.3111 | 100.00 |
| 13.5391 | 25.04 |
| 14.0837 | 34.93 |
| 14.5855 | 33.39 |
| 15.3831 | 8.76 |
| 15.5724 | 12.24 |
| 15.9676 | 9.12 |
| 16.7336 | 64.64 |
| 17.4857 | 6.14 |
| 18.0702 | 31.51 |
| 18.3862 | 8.90 |
| 19.2183 | 16.27 |
| 20.0081 | 39.14 |
| 20.3419 | 26.48 |
| 21.1256 | 34.24 |
| 21.3242 | 15.79 |
| 22.0092 | 35.62 |
| 22.5028 | 16.08 |
| 23.1445 | 7.75 |
| 23.4107 | 11.70 |
| 23.8241 | 9.17 |
| 24.3918 | 19.32 |
| 24.5913 | 18.26 |
| 24.9140 | 46.75 |
| 25.3974 | 32.79 |
| 25.5768 | 43.71 |
| 26.1570 | 11.50 |
| 26.7323 | 3.55 |
| 27.2280 | 21.80 |
| 27.5416 | 32.47 |
| 27.8348 | 16.14 |
| 28.0704 | 8.75 |
| 28.6818 | 11.22 |
| 29.3712 | 4.98 |
| 30.3808 | 4.04 |
| 31.2917 | 10.24 |
| 31.5862 | 11.98 |
| 32.9442 | 5.01 |
| 33.6350 | 4.99 |
| 33.9874 | 2.68 |
| 34.4781 | 3.01 |
| 34.8120 | 4.21 |
| 35.6513 | 3.06 |
| 37.1454 | 3.83 |
| 38.9841 | 1.18 |
| 39.4671 | 1.81 |
| 40.6150 | 4.58 |
| 42.5268 | 2.93 |
| 43.4580 | 2.63 |
| 44.1621 | 1.20 |
| 45.6961 | 2.04 |
| 46.7044 | 4.03 |
| 48.7494 | 8.95 |
| 48.8885 | 4.57 |
| 49.8753 | 4.63 |

### Example 2: Preparation of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A) Monohydrate, Seed Material

Approx. 200 mg of Compound A hydrate obtained by Example 1 was added to 400-800 μl of either ethyl acetate or isopropyl acetate and the resulting suspension stirred at 60° C. for 5 days. The precipitate was then filtered and dried under vacuum at 50° C. for 24 hours to yield crystalline monohydrate of Compound A.

### Example 3: Preparation of Crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A) Monohydrate

Compound A hydrate (100 g) obtained by a procedure analogous to the synthesis method as described in Example 158 of WO 2018/119036 was charged in a flask (R1) together with ethanol (150-170 mL) and ethyl acetate (80-100 mL). The obtained mixture was heated to 400-50° C. and stirred for 0.5-2 hours. Water (4-7 mL) was then added and the water content was measured by Karl Fischer titration. The content of R1 was warmed to 40°-55° C. and filtered into a second flask (R2) pre-heated at 40°-55° C. R1 was rinsed with ethyl acetate (80-100 mL) at 40°-50° C. and The content filtered into R2. n-Heptane (340-410 mL) was charged into R2 in about 20-40 min. maintaining 40°-55° C. The obtained solution was seeded with 1.9-2.1 g of crystalline monohydrate of Compound A and the obtained mixture was stirred at 40°−55° C. for 4-8 hours. n-heptane (680-750 mL) was added in 10-15 hours maintaining 400-55° C.; the obtained mixture was stirred for additional 2-5 hours at 40°-55° C., then it was cooled down to 20°-25° C. for 7-13 hours. The suspension was stirred at 20°-25° C. for 12-18 h, then it was filtered and washed with n-Heptane (180-250 mL). After drying under vacuum at 45°-55° C. for 15-22 hours, crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide monohydrate was obtained with an 80% yield.

### Example 3b: Alternative Preparation of Crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A) Monohydrate

Compound A hydrate (25 g) obtained by a procedure analogous to the synthesis method as described in Example 158 of WO 2018/119036 was charged in a flask (R1) together with water (2.5-4.5 mL) and isopropyl alcohol (IPA) (100 mL). The obtained mixture was heated to 50° C. and stirred for 0.5-2 hours. n-Heptane (125 mL) was charged into R1. The obtained solution was seeded with 500 mg of crystalline monohydrate of Compound A and the obtained mixture was Stirred at 50° C. for 72 hours. n-Heptane (275 mL) was added in 12 hours maintaining 50° C.; the obtained mixture was stirred for additional 58 hours at 50° C., then it was cooled down to 200-25° C. for 2 hours. The suspension was stirred at 20°-25° C. for 94 h, then it was filtered and washed with n-heptane (100 mL). After drying under vacuum at 50° C. for 24 hours, crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide monohydrate was obtained with an 90% yield.

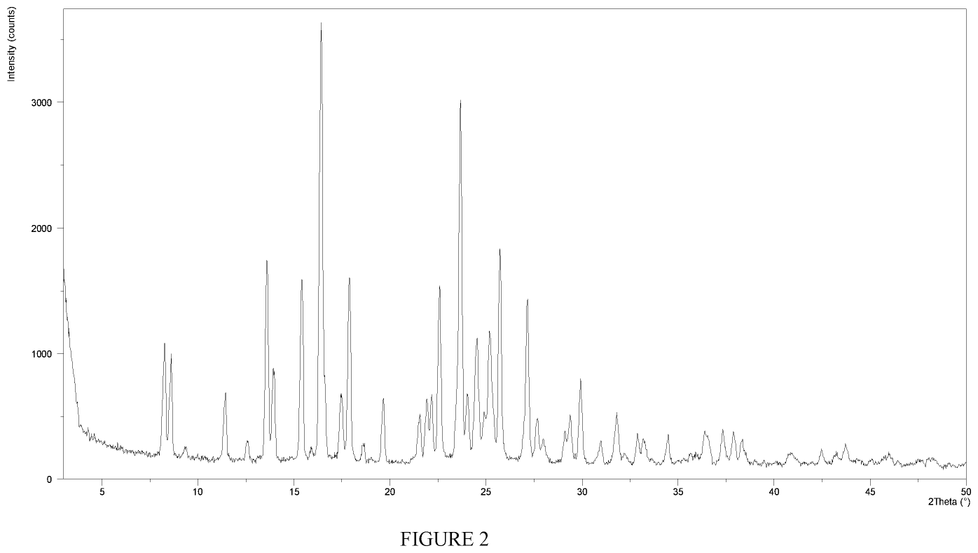

The crystalline monohydrate (as obtained by Example 3 or 3b) was characterized by XRPD (see FIG. 2). Table 2 provides peak listings and relative intensities for the XPRD.

TABLE 2

| Pos. [°2Th.] | Rel. Int. [%] |
|---|---|
| 8.2904 | 25.26 |
| 8.6250 | 23.96 |
| 9.3485 | 2.24 |
| 11.4511 | 14.20 |
| 12.5682 | 4.31 |
| 13.6202 | 45.95 |
| 13.9754 | 21.49 |
| 15.4397 | 41.22 |
| 15.8867 | 3.10 |
| 16.4426 | 100.00 |
| 16.6283 | 17.71 |
| 17.5110 | 14.58 |
| 17.9121 | 41.41 |
| 18.6250 | 4.18 |
| 19.6673 | 14.48 |
| 21.5675 | 11.28 |
| 21.9258 | 14.96 |
| 22.1775 | 15.69 |
| 22.5940 | 41.75 |
| 23.6809 | 85.80 |
| 24.0437 | 15.69 |
| 24.5412 | 27.75 |
| 25.1642 | 29.90 |
| 25.7310 | 49.96 |
| 27.1482 | 38.49 |
| 27.6772 | 10.70 |
| 27.9857 | 5.32 |
| 29.0996 | 7.66 |
| 29.3985 | 10.88 |
| 29.9267 | 20.17 |
| 30.9874 | 5.22 |
| 31.8056 | 12.06 |
| 32.8799 | 7.23 |
| 33.1991 | 5.73 |
| 34.4861 | 6.97 |
| 36.3854 | 7.95 |
| 36.6246 | 4.89 |
| 37.3258 | 7.90 |
| 37.8748 | 7.87 |
| 38.3143 | 5.55 |
| 40.8261 | 2.60 |
| 42.4567 | 3.57 |
| 43.2056 | 2.48 |
| 43.7464 | 4.48 |
| 45.0366 | 1.28 |
| 46.0177 | 2.48 |
| 48.3545 | 1.47 |

Compound A can be used as starting material in the process to prepare a pharmaceutical formulation as described herein, as obtained in Example 1 (i.e. a crystalline hydrate form), as obtained in Example 3 or Example 3b (i.e. a crystalline monohydrate form), or in any other form.

### Example 4: Solubility of the API in the Second Component

The solubility of an API in the second component may be obtained using hot stage microscopy or differential scanning microscopy (DSC). First, the API may be added to molten second component at various concentrations, covering a range below and above the solubility limit of the API in the molten matrix

Hot stage microscopy method: Solidified samples of the API at various concentrations in the second component, which have been stored for a period of time at a certain temperature condition, may be heated from room temperature to a temperature above the second component drop point at different heating rates (e.g. 3° C./min, 10° C./min and 30° C./min). The highest concentration with no visible crystals is considered as the closest approximation of the thermodynamic solubility at a particular storage temperature.

DSC method: Samples of the API at various concentrations in molten second component (above and below the solubility in the matrix) may be poured into DSC pan, in a sample holder together with an empty reference pan), and allowed to solidify. Samples may be measured at different heating rates (e.g. 3° C./min, 5° C./min and 10° C./min), heating from 25° C. to a temperature above the drop point of the second component. Software may then be used to integrate the DSC curve to obtain the enthalpy change for each sample concentration. Saturation solubility can be obtained from a graph of sample concentration versus enthalpy change and is the point at which enthalpy is lowest.

### Example 5: Process for Preparing a Compound a Stearoyl Polyoxyl-32 Glycerides Formulation

Stearoyl polyoxyl-32 glycerides (Gelucire® 50/13) and, where present, all-rac-alpha-Tocopherol (vitE) were dispensed, melted and mixed successively into a suitable container at 60° C.±5° C. Compound A monohydrate (obtained from Example 3) was dispensed and mixed with constant stirring the molten mixture under nitrogen blanketing until a homogeneous solution was formed. The obtained molten mixture was manually filled into hard gelatin or HPMC capsules using a positive displacement pipette. Alternatively, for larger batch sizes (e.g. >100 units), the bulk solution can be transferred into the capsule filling machine hopper pre-heated to 60° C.±5° C. followed by filling into hard gelatin or HPMC capsules. The filled capsules were collected and stored at room temperature. The filled capsules can be stored in LDPE bags in suitable containers until packaging in HDPE bottles. The capsules can be packed in a HDPE bottle followed by induction sealing. After filling the capsules can be controlled for appearance and weight. During bottling the number of capsules can be counted and after bottling the bottles can be checked for seal integrity.

Compound A was supplied in the monohydrate form as starting material in an amount equivalent with 50 mg, 100 mg, 150 mg and 200 mg of anhydrous Compound A in the final hard gelatin or HPMC capsules for oral administration.

Tables 3 and 4 provide exemplary component quantities for Compound A stearoyl polyoxyl-32 glycerides capsule formulations.

TABLE 3

| | 50 mg/cap | | 100 mg/cap | | 150 mg/cap | | 200 mg/cap | |
|---|---|---|---|---|---|---|---|---|
| Component | (mg) | (%) | (mg) | (%) | (mg) | (%) | (mg) | (%) |
| Compound A* | 52.00 | 22.81 | 104.00 | 22.81 | 156.00 | 22.88 | 208.00 | 22.88 |
| monohydrate | | | | | | | | |
| Gelucire ® | 175.25 | 77.11 | 350.50 | 77.11 | 525.75 | 77.11 | 701.00 | 77.11 |
| 50/13 | | | | | | | | |
| Vitamin E | 0.02 | 0.01 | 0.05 | 0.01 | 0.07 | 0.01 | 0.09 | 0.01 |
| Total | 227.27 | 100.00 | 454.55 | 100.00 | 681.82 | 100.00 | 909.09 | 100.00 |
| Gelatin capsule | Size 3 | Size 3 | Size 1 | Size 1 | Size 00 | Size | Size | Size |
| | | | | | | 00 | 00 | 00 |

*Compound A is used in the monohydrate form as starting material. The amount of Compound A monohydrate is calculated based on the active anhydrous equivalent in the final formulation, where a conversion factor from the anhydrous form to the monohydrate is 1.04.

TABLE 4

| | 50 mg/cap | | 100 mg/cap | | 150 mg/cap | |
|---|---|---|---|---|---|---|
| Component | (mg) | (%) | (mg) | (%) | (mg) | (%) |
| Compound A* | 52.00 | 17.93 | 104.00 | 17.93 | 156.00 | 17.93 |
| monohydrate | | | | | | |
| Gelucire ® 50/13 | 237.97 | 82.06 | 475.95 | 82.06 | 713.92 | 82.06 |
| Vitamin E | 0.03 | 0.01 | 0.06 | 0.01 | 0.09 | 0.01 |
| Total | 290.00 | 100.00 | 580.00 | 100.00 | 870.00 | 100.00 |
| Gelatin capsule | Size 1 | Size 1 | Size 0 | Size 0 | Size 00 | Size 00 |

*Compound A is used in the monohydrate form as starting material. The amount of Compound A monohydrate is calculated based on the active anhydrous equivalent in the final formulation, where a conversion factor from the anhydrous form to the monohydrate is 1.04.

### Example 6: Process for Preparing Capsules of a Compound a Lauroyl Polyoxyl-32 Glycerides Formulation

Lauroyl polyoxyl-32 glycerides (Gelucire® 44/14) was dispensed and melted into a suitable container at 60° C.±5° C. All-rac-alpha-Tocopherol (vitE) was dispensed and mixed with the molten lauroyl polyoxyl-32 glycerides until homogeneous. Compound A monohydrate (obtained from Example 3) was dispensed and mixed with constant stirring the molten mixture under nitrogen blanketing until a homogeneous solution was formed. The obtained molten mixture was manually filled into hard gelatin or HPMC capsules using a positive displacement pipette. Alternatively, for larger batch sizes (e.g. >100 units), the bulk solution can be transferred into the capsule filling machine hopper pre-heated to 60° C.±5° C. followed by filling into hard gelatin or HPMC capsules. The filled capsules were collected and stored at room temperature. The filled capsules can be stored in LDPE bags in suitable containers until packaging in HDPE bottles. The capsules can be packed in a HDPE bottle followed by induction sealing. After filling the capsules can be controlled for appearance and weight. During bottling the number of capsules can be counted and after bottling the bottles can be checked for seal integrity.

The exemplary component quantities for Compound A stearoyl polyoxyl-32 glycerides formulations (Tables 3 and 4 above) may be used for Compound A lauroyl polyoxyl-32 glycerides formulations, by replacing the stearoyl polyoxyl-32 glycerides (Gelucire® 50/13) in Tables 3 and 4 with lauroyl polyoxyl-32 glycerides (Gelucire® 44/14).

### Example 7: Process for Preparing Capsules of a Compound a Polyoxyl-32 Stearate Formulation

Polyoxyl-32 stearate (type 1) (Gelucire® 48/16) and, where present, all-rac-alpha-Tocopherol (vitE), were dispensed, melted and mixed successively into a suitable container at 60° C.±5° C. Compound A monohydrate (obtained from Example 3) was dispensed and mixed into the molten mixture under nitrogen blanketing until Compound A monohydrate was completely dissolved. The obtained molten mixture was manually filled into hard gelation or HPMC capsules using a positive displacement pipette. Alternatively, for larger batch sizes (e.g. >100 units), the bulk solution can be transferred into the capsule filling machine hopper pre-heated to 60° C.±5° C. followed by filling into hard gelatin or HPMC capsules. The filled capsules were collected and stored at room temperature. The filled capsules can be stored in LDPE bags in suitable containers until packaging in HDPE bottles. The capsules can be packed in a HDPE bottle followed by induction sealing. After filling the capsules can be controlled for appearance and weight. During bottling the number of capsules can be counted and after bottling the bottles can be checked for seal integrity.

Compound A was supplied in the monohydrate form as starting material in an amount equivalent with 50, 100, 150 and 200 mg of anhydrous Compound A in the final hard gelatin or HMPC capsules for oral administration.

Compound A polyoxyl-32 stearate formulations containing a crystallization rate inhibitor (HPMC or PVPVA) were also prepared. HPMC and PVPVA both form suspensions in the molten mixture. Polyoxyl-32 stearate (type 1) and, where present, all-rac-alpha-Tocopherol (vitE), was dispensed and melted successively into a suitable container at 60° C.±5° C. Compound A monohydrate (obtained from Example 3) was dispensed and mixed into the molten mixture under nitrogen blanketing until Compound A monohydrate was completely dissolved. HPMC or PVPVA was added at 60° C.±5° C. to form a fully dispersed suspension.

A crystallization rate inhibitor that is soluble in the mixture may also be used. In this case, it may be added to the melted polyoxyl-32 stearate (type 1) prior to addition of Compound A monohydrate, to effect complete solubilization at 60° C.±5° C.

Dispersions of the following crystallization rate inhibitors in polyoxyl-32 stearate type 1 were prepared: 1% or 5% PVP (Plasdone® K-12) in polyoxyl-32 stearate; 1% or 5% polyethylene glycol-polyvinyl acetate-polyvinylcaprolactame-based graft copolymer (Soluplus®) in polyoxyl-32 stearate; 5% HPMCAS LG, hydroxylpropylcellulose (Klucel™ ELF PHARM) in polyoxyl-32 stearate; 1% poly(vinyl alcohol) (Mowiol® 8-88) in polyoxyl-32 stearate; and 1% hydroxyethylcellulose (Natrosol™ 250L PHARM) in polyoxyl-32 stearate.

Tables 5 and 6 provide exemplary component quantities for Compound A polyoxy-32 stearate type I capsule formulations.

TABLE 5

| | 50 mg/cap | | 100 mg/cap | | 150 mg/cap | | 200 mg/cap | |
|---|---|---|---|---|---|---|---|---|
| Component | (mg) | (%) | (mg) | (%) | (mg) | (%) | (mg) | (%) |
| Compound A* | 52.00 | 23.85 | 104.00 | 23.85 | 156.00 | 23.85 | 208.00 | 23.85 |
| monohydrate | | | | | | | | |
| Gelucire ® | 165.98 | 76.14 | 331.96 | 76.14 | 497.93 | 76.14 | 663.91 | 76.14 |
| 48/16 | | | | | | | | |
| Vitamin E | 0.02 | 0.01 | 0.04 | 0.01 | 0.07 | 0.01 | 0.09 | 0.01 |
| Total | 218.00 | 100.00 | 436.00 | 100.00 | 654.00 | 100.00 | 872.00 | 100.00 |
| HPMC capsule | Size 3 | Size 3 | Size 0 | Size 0 | Size | Size | Size | Size |
| | | | | | 00 | 00 | 00 | 00 |

*Compound A is used in the monohydrate form as starting material. The amount of Compound A monohydrate is calculated based on the active anhydrous equivalent in the final formulation, where a conversion factor from the anhydrous form to the monohydrate is 1.04.

TABLE 6

| | 200 mg/cap | | 200 mg/cap | | 200 mg/cap | |
|---|---|---|---|---|---|---|
| Component | (mg) | (%) | (mg) | (%) | (mg) | (%) |
| Compound A * | 208.00 | 23.85 | 208.00 | 23.85 | 208.00 | 23.85 |
| monohydrate | | | | | | |
| Gelucire ® 48/16 | 655.22 | 75.14 | 646.50 | 74.14 | 637.78 | 73.14 |
| HPMC 3 cps | 8.72 | 1.00 | 17.44 | 2.00 | 26.16 | 3.00 |
| Vitamin E | 0.09 | 0.01 | 0.09 | 0.01 | 0.09 | 0.01 |
| Total | 872.00 | 100.00 | 872.00 | 100.00 | 872.00 | 100.00 |
| HPMC capsule | Size 00 | Size 00 | Size 00 | Size 00 | Size 00 | Size 00 |

*Compound A is used in the monohydrate form as starting material. The amount of Compound A monohydrate is calculated based on the active anhydrous equivalent in the final formulation, where a conversion factor from the anhydrous form to the monohydrate is 1.04.

### Example 8: Pharmacokinetics of Compound a after Single Oral Administration at 200 Mg in Fasted and Fed Dogs

A pharmacokinetic (PK) study was carried out using a Compound A stearoyl polyoxy-32 glycerides formulation.

A formulation of compound A and stearoyl polyoxy-32 glycerides (Gelucire® 50/13) in size 00 hard gelatin capsules (200 mg dose per capsule), according to Table 3, was administered orally to male beagle dogs (N=3) in a fasted state as well as a fed state.

TABLE 7
Mean (SD) Plasma Pharmacokinetics of Compound A in Male Dogs following a
200 mg oral dose

| Feeding | Cₘₐₓ | tₘₐₓ^{a} | AUC_{0-∞} | AUC_{0-96 h} | AUC_{0-24 h} | | Fed vs |
|---|---|---|---|---|---|---|---|
| Regimen | (ng/mL) | (h) | (ng · h/mL) | (ng · h/mL) | (ng · h/mL) | t_{1/2} (h) | Fasted |
| Fasted | 16000 | 2.0 | 578000 | 521000 | 246000 | 30^{c} | NA |
| | (6610) | (1.0-4.0) | (276000) | (223000) | (117000) | (NC) | (NA) |
| Fed | 9330 | 24 | NC^{d} | 502000 | 121000 | 34^{c} | 1.3 |
| | (2310) | (24-24) | (NC) | (289000) | (77200) | (NC) | (1.4) |

AUC = area under the plasma concentration-time curve; AUC_{0-24 h} = AUC from time 0 to 24 hours post-dose; AUC_{0-96 h} = AUC from time 0 to 96 hours post-dose; AUC_{0-∞} = AUC from time 0 to infinity with extrapolation of the terminal phase; Cₘₐₓ = maximum observed plasma concentration; t_{1/2} = half-life; tₘₐₓ = time correspondent to the maximum observed plasma concentration; SD = standard deviation, given in brackets for columns 2 and 4-8.
^{a}Median (Min-Max);
^{c}N = 2, poor linear regression;
^{d}Not Calculated, AUCextrapolation exceeds 20%; NA: Not Available, this formulation was used as the reference group.

The pharmacokinetic data clearly indicated that the AUC₀₋₉₆ₕ was similar for fasted and fed state, indicating the absence of a food effect. Hence, this formulation releases the API independent of the presence or absence of the food. This is beneficial from a patient compliance point of view.

Therefore, a formulation according to the invention may result in a reduced food effect compared to other formulations.

### Example 9—Pharmacokinetics of Compound a after Single Oral Administration at 200 and 600 mg as Different Capsule Formulations in Fasted Dogs

A pharmacokinetic (PK) study was carried out using various capsule formulations of Compound A. The aim of this study was to understand the effect of high dose on the pharmacokinetics of the API in the different formulations. A 200 mg or 600 mg dose was administered orally to fasted male beagle dogs (N=3/group) in a crossover manner. The study included three groups with an appropriate washout period in between, i.e. 5 plasma-half-lives. Three formulations were:
Group 1—Solid Dosage Forms—capsules: polyoxyl-32 stearate type I (Gelucire® 48/16)—Size 00 HPMC capsules—200 mg. 1 capsule dosed—total dose 200 mg.
Group 2—Solid Dosage Forms—capsules: stearoyl polyoxy-32 glycerides (Gelucire® 50/13)—Size 00 hard gelatin capsule—150 mg. 4 capsules dosed—total dose 600 mg
Group 3—Solid Dosage Forms—capsules: polyoxyl-32 stearate type I (Gelucire® 48/16)—Size 00 HPMC capsules—200 mg. 3 capsules dosed—total dose 600 mg.

### Polyoxyl-32 Stearate Type I (Gelucire® 48/16) 200 mg Capsules

20 dosage units were prepared.

### Quantity Per Dosing Unit:

Gelucire® 48/16: 664 mg
Compound A monohydrate*: 208 mg *Compound A is used in the monohydrate form as starting material. The amount of Compound A monohydrate is calculated based on the active anhydrous equivalent in the final formulation, where a conversion factor from the anhydrous form to the monohydrate is 1.04.
Capsule: size 00 HMPC

### Stearoyl Polyoxy-32 Glycerides (Gelucire® 50/13) 150 mg Capsules

25 dosage units were prepared.

### Quantity Per Dosing Unit:

Gelucire 50/13®: 714.0 mg
Compound A monohydrate*: 156.0 mg
Capsule: size 00 hard gelatin

TABLE 8
Mean (SD) Plasma Pharmacokinetics of Compound A in Male Dogs Following
Single Oral Dosing

| | | Total | | | | | |
|---|---|---|---|---|---|---|---|
| | | Dose | Cₘₐₓ | tₘₐₓ^{a} | AUC_{0-∞} | AUC₀₋₉₆ | AUC₀₋₂₄ |
| Group | Formulation | (mg) | (ng/mL) | (h) | (ng · h/mL) | (ng · h/mL) | (ng · h/mL) |
| 1 | Gelucire 48/16 | 200 | 8690 | 2.0 | 281000 | 274000 | 139000 |
| | (1 × 200 mg) | | (3190) | (1.0-7.0) | (38400) | (34900) | (37300) |
| 2 | Gelucire 50/13 | 600 | 31900 | 4.0 | 1580000^{b} | 1600000 | 592000 |
| | (4 × 150 mg) | | (5840) | (4.0-7.0) | (NC) | (399000) | (91600) |
| 3 | Gelucire 48/16 | 600 | 24700 | 1.0 | 819000 | 763000 | 354000 |
| | (3 × 200 mg) | | (7290) | (1.0-1.0) | (373000) | (342000) | (124000) |

AUC = area under the plasma concentration-time curve; AUC_{0-24 h} = AUC from time 0 to 24 hours post-dose; AUC_{0-96 h} = AUC from time 0 to 96 hours post-dose; AUC_{0-∞} = AUC from time 0 to infinity with extrapolation of the terminal phase; Cₘₐₓ = maximum observed plasma concentration; tₘₐₓ = time correspondent to the maximum observed plasma concentration; SD = standard deviation, given in brackets for Cₘₐₓ and AUC.
^{a}Median (Min-Max);
^{b}N = 2.

The results indicate that the stearoyl polyoxy-32 glycerides (Gelucire® 50/13) formulation with 600 mg (4*150 mg capsule) dose shows very good AUC and almost complete absorption. This demonstrates that the presence of stearoyl polyoxy-32 glycerides can keep the drug in supersaturated state even at a dose of 600 mg.

### Example 10—Physiology-Based Dissolution Test of Compound a Polyoxyl-32 Stearate Formulations

A physiology-based dissolution test (PBDT) was carried out using HPMC or hard gelatin capsule formulations of Compound A polyoxyl-32 stearate type I (Gelucire® 48/16), prepared according to Example 7. The component quantities of the capsule formulations tested are set out in Tables 9 and 10.

PBDT in FaSSIF medium was performed using a USP type 2 paddle apparatus at 75 rpm, according to a two step procedure. In the first step, 300 mL of simulated gastric fluid sine pepsine pH 1.3 was used. In the second step, 600 mL of concentrated simulated intestinal fluid (added after 15 mins of step 1) was added taking the total dissolution medium volume to 900 mL and a pH of 6.5. The amount of compound A present in the dissolution medium is analysed using high performance liquid chromatography with a UV detector. The time points for the analysis were 5, 10, 14, 20, 25, 30, 45, 60, 75, 105, and 135 minutes post the sample is introduced in to the vessel. In order to maintain the capsules at the bottom of the dissolution vessel, “closed-4 spiral sinker 29.2/11.8” were used.

TABLE 9
HPMC capsules

| | Approx. 12% | | Approx. 24% | | Approx. 24% | | Approx. 30% | |
|---|---|---|---|---|---|---|---|---|
| | DL, | | DL, | | DL, | | DL, | |
| | 100 mg/cap | | 100 mg/cap | | 200 mg/cap | | 100 mg/cap | |
| Component | (mg) | (%) | (mg) | (%) | (mg) | (%) | (mg) | (%) |
| Compound A* | 104 | 11.93 | 104 | 23.85 | 208 | 23.85 | 104 | 29.71 |
| monohydrate | | | | | | | | |
| Gelucire ® | 767.91 | 88.06 | 331.96 | 76.14 | 663.91 | 76.14 | 245.97 | 70.28 |
| 48/16 | | | | | | | | |
| Total | 872 | 100 | 436 | 100 | 872 | 100 | 350 | 100 |

| | | | | |
|---|---|---|---|---|
| Capsule size | Size 00 | Size 1 | Size 1 | Size 1 |

DL = drug loading.
*Compound A is used in the monohydrate form as starting material. The amount of Compound A monohydrate is calculated based on the active anhydrous equivalent in the final formulation, where a conversion factor from the anhydrous form to the monohydrate is 1.04.

TABLE 10
Hard gelatin capsules

24% DL,
100 mg/cap

| Component | (mg) | (%) |
|---|---|---|
| Compound A* | 104 | 23.85 |
| monohydrate | | |
| Gelucire ® 48/16 | 331.96 | 76.14 |
| Total | 436 | 100 |

| | |
|---|---|
| Capsule | Size 1 |

DL = drug loading.
*Compound A is used in the monohydrate form as starting material. The amount of Compound A monohydrate is calculated based on the active anhydrous equivalent in the final formulation, where a conversion factor from the anhydrous form to the monohydrate is 1.04.

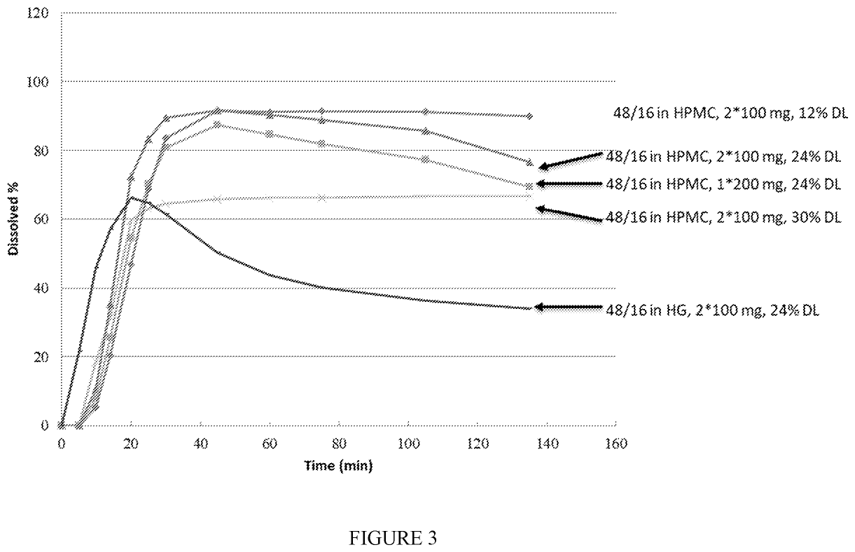

The dissolution results for each of the formulations tested are shown in FIG. 3. In FIG. 3, “48/16” refers to Gelucire® 48/16 (i.e. polyoxyl-32 stearate type I); “DL” refers to drug load; “HG” refers to hard gelatin capsules; “HPMC” refers to HPMC capsules; and 2*100 mg, for example, refers to 2 units of a 100 mg capsule, wherein 100 mg is the amount of drug per capsule.

Based on this study, it can be concluded that Compound A polyoxyl-32 stearate type I formulations in a HPMC capsule perform superiorly to the same formulations in a gelatin capsule. It is postulated that the HPMC from the HPMC capsule acts as a crystallization inhibitor. Moreover, there is close to 100% drug release for all the drug loads (from 12% to 30%); however, the level of supersaturation varies based on drug load. Higher drug loads have a latent drug precipitation and lower drug loads have a stable supersaturation.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention and that embodiments within the scope of these claims and their equivalents be covered thereby.

1. A pharmaceutical formulation, comprising a first component and a second component;
wherein the first component is an active pharmaceutical ingredient which is a compound of Formula (I) 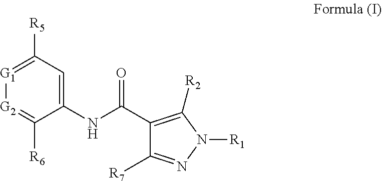
wherein
R₁ is selected from the group consisting of
i) naphthalen-1-yl, optionally substituted with a fluoro or amino substituent;
and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
R₂ is selected from the group consisting of C₁₋₄alkyl, 1-methoxy-ethyl, difluoromethyl, fluoro, chloro, bromo, cyano, and trifluoromethyl;
G₁ is N or C(R₄);
G₂ is N or C(R₃); such that only one of G₁ and G₂ are N in any instance;
R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy;
and
xviii) morpholin-4-ylcarbonyl;
R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
R₆ is hydrogen, C₁₋₄alkyl, fluoro, 2-methoxy-ethoxy, chloro, cyano, or trifluoromethyl; and
R₇ is hydrogen or fluoro;
or an enantiomer, diastereomer, solvate, or pharmaceutically acceptable salt form thereof; and
wherein the second component is a mixture comprising fatty acid and polyethylene glycol monoesters and diesters, and optionally, fatty acid and glycerol monoesters, diesters and triesters;
wherein the fatty acid component of the fatty acid and polyethylene glycol monoesters and diesters, and of the fatty acid and glycerol monoesters, diesters and triesters, when present, comprises one or more saturated fatty acids having at least eight carbons.

2. The pharmaceutical formulation of claim 1, wherein the fatty acid component of the fatty acid and polyethylene glycol monoesters and diesters comprises stearic acid and optionally palmitic acid.

3. (canceled)

4. The pharmaceutical formulation of claim 2, wherein the second component is substantially free of fatty acid and glycerol monoesters, diesters and triesters.

5. The pharmaceutical formulation of claim 1, wherein the second component comprises a mixture of fatty acid and polyethylene glycol monoesters and diesters, and fatty acid and glycerol monoesters, diesters and triesters.

6. The pharmaceutical formulation of claim 5, wherein the fatty acid component of the fatty acid and polyethylene glycol monoesters and diesters and of the fatty acid and glycerol monoesters, diesters and triesters comprises stearic acid and optionally palmitic acid.

7. (canceled)

8. (canceled)

9. The pharmaceutical formulation of claim 1, wherein the second component further comprises free polyethylene glycol.

10. The pharmaceutical formulation of claim 1, wherein the pharmaceutical formulation is a solid or semi-solid formulation.

11. The pharmaceutical formulation of claim 1, wherein the second component has a drop point of at least about 30° C.

12. (canceled)

13. (canceled)

14. The pharmaceutical formulation of claim 1, wherein the polyethylene glycol has an average molecular weight of from about 250 g/mol to about 5000 g/mol.

15. (canceled)

16. The pharmaceutical formulation of claim 1, wherein the polyethylene glycol is PEG-32.

17. The pharmaceutical formulation of claim 1, wherein the formulation further comprises an antioxidant, or a crystallisation rate inhibitor.

18. The pharmaceutical formulation of claim 17, wherein the antioxidant is selected from tocopherol (vitamin E), thiodipropionic acid, lipoic acid, hydroquinone, phytic acid, monothioglycerol, sodium thioglycolate, thioglycol, beta carotene, butylated hydroxyanisole (BHA), butylated hydroxytoluene (BHT), cysteine, cysteine hydrochloride, propyl gallate (PG), sodium metabisulfite, ascorbyl palmitate, ascorbyl stearate, potassium metabisulfite, disodium EDTA (ethylenediamine tetraacetic acid; also known as disodium edentate), EDTA, erythorbic acid, ethoxyquin, glutathione, gum guaiac, lecithin, propyl gallate, TBHQ (tert butyl hydroxyquinone), tartaric acid, citric acid, citric acid monohydrate, methane sulfonic acid, methionine, sodium metabisulfite, sodium thiosulfate, sodium sulphite, and combinations thereof.

19. (canceled)

20. (canceled)

21. The pharmaceutical formulation of claim 1, wherein the formulation comprises from about 0.1 w/w % to about 40 w/w %, from about 1 w/w % to about 30 w/w %, from about 5 w/w % to about 25 w/w %, or from about 12 w/w % to about 25 w/w % of the active pharmaceutical ingredient relative to the total weight of the formulation.

22. (canceled)

23. (canceled)

24. The pharmaceutical formulation of claim 23, wherein the crystallisation rate inhibitor is selected from polyvinylpyrrolidone (PVP), a polyvinylpyrrolidone-vinyl acetate copolymer (PVPVA), a poly(meth)acrylate polymer, a cyclodextrin and a derivative thereof, hydroxypropylcellulose, hydroxyethylcellulose methylcellulose, hydroxypropyl methylcellulose (HPMC), hydroxypropyl methylcellulose acetate succinate (HPMCAS), poly(vinyl alcohol), a poloxamer, and combinations thereof.

25. (canceled)

26. The pharmaceutical formulation of claim 23, wherein the formulation comprises from about 0.5 w/w % to about 15 w/w % of the crystallisation rate inhibitor relative to the total weight of the formulation.

27. (canceled)

28. The pharmaceutical formulation according to claim 1, wherein the compound of Formula (I) is
1-(1-oxo-1,2-dihydroisoquinolin-5-yl)-5-(trifluoromethyl)-N-(2-(trifluoromethyl)pyridin-4-yl)-1H-pyrazole-4-carboxamide: 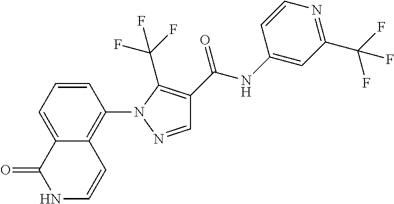 [SMILES: O=C(Nc1ccnc(C(F)(F)F)c1)c1cnn(-c2cccc3c(=O)[nH]ccc23)c1C(F)(F)F]
or a solvate or pharmaceutically acceptable salt thereof.

29. A solid dosage form comprising the pharmaceutical formulation of claim 1.

30. (canceled)

31. The solid dosage form of claim 29, wherein the formulation comprises from about 2 mg to about 1000 mg of the active pharmaceutical ingredient.

32. The solid dosage form of claim 29, wherein the formulation comprises 50, 100, 150 or 200 mg of
1-(1-oxo-1,2-dihydroisoquinolin-5-yl)-5-(trifluoromethyl)-N-(2-(trifluoromethyl)pyridin-4-yl)-1H-pyrazole-4-carboxamide: 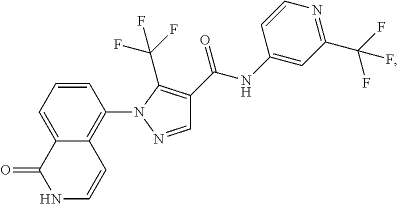 [SMILES: O=C(Nc1ccnc(C(F)(F)F)c1)c1cnn(-c2cccc3c(=O)[nH]ccc23)c1C(F)(F)F]
or a solvate or pharmaceutically acceptable salt thereof, calculated based on the free base form.

33. The solid dosage form of claim 29,
wherein the dosage form comprises a capsule encapsulating the pharmaceutical formulation.

34. The solid dosage form of claim 29, wherein the dosage form comprises a hard capsule encapsulating the pharmaceutical formulation.

35. The solid dosage form of claim 34, wherein the hard capsule is a gelatin capsule or a hydroxypropyl methylcellulose (HPMC) capsule.

36. A method of treating a disease, syndrome, condition, or disorder, wherein said disease, syndrome, condition, or disorder is affected by the inhibition of MALT1, comprising administering to a subject in need thereof a therapeutically effective amount of a pharmaceutical formulation of claim 1.

37. The method of claim 36 wherein said disease, syndrome, condition, or disorder is selected from cancer and immunological diseases.

38. (canceled)

39. (canceled)

40. A process for preparing a solid or semi-solid pharmaceutical formulation, comprising the steps of:
a) forming a melt comprising a first component and a second component, wherein
the forming a melt step comprises heating the second component; and
b) cooling the melt;
to provide a solid or semi-solid pharmaceutical formulation;
wherein the first component is an active pharmaceutical ingredient which is a compound of Formula (I) 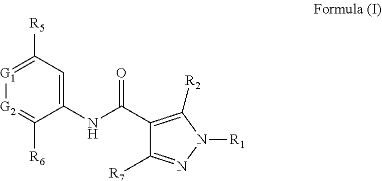
wherein
R₁ is selected from the group consisting of
i) naphthalen-1-yl, optionally substituted with a fluoro or amino substituent;
and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
R₂ is selected from the group consisting of C₁₋₄alkyl, 1-methoxy-ethyl, difluoromethyl, fluoro, chloro, bromo, cyano, and trifluoromethyl;
G₁ is N or C(R₄);
G₂ is N or C(R₃); such that only one of G₁ and G₂ are N in any instance;
R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy;
and
xviii) morpholin-4-ylcarbonyl;
R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
R₆ is hydrogen, C₁₋₄alkyl, fluoro, 2-methoxy-ethoxy, chloro, cyano, or trifluoromethyl; and
R₇ is hydrogen or fluoro;
or an enantiomer, diastereomer, solvate, or pharmaceutically acceptable salt form thereof; and
wherein the second component is a mixture comprising fatty acid and polyethylene glycol monoesters and diesters, and optionally, fatty acid and glycerol monoesters, diesters and triesters;
wherein the fatty acid component of the fatty acid and polyethylene glycol monoesters and diesters, and of the fatty acid and glycerol monoesters, diesters and triesters, when present, comprises one or more saturated fatty acids having at least eight carbons.

41. The process of claim 40, wherein the fatty acid component of the fatty acid and polyethylene glycol monoesters and diesters comprises stearic acid and optionally palmitic acid.

42. A process for preparing a solid dosage form, the process comprising the steps of:
a) forming a melt comprising a first component and a second component, wherein
the forming a melt step comprises heating the second component;
b) filling a capsule with the melt; and
c) cooling the filled capsule;
to provide a solid dosage form;
wherein the active pharmaceutical ingredient is an active pharmaceutical ingredient which is a compound of Formula (I) 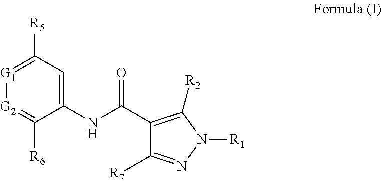
wherein
R₁ is selected from the group consisting of
i) naphthalen-1-yl, optionally substituted with a fluoro or amino substituent;
and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
R₂ is selected from the group consisting of C₁₋₄alkyl, 1-methoxy-ethyl, difluoromethyl, fluoro, chloro, bromo, cyano, and trifluoromethyl;
G₁ is N or C(R₄);
G₂ is N or C(R₃); such that only one of G₁ and G₂ are N in any instance;
R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy;
and
xviii) morpholin-4-ylcarbonyl;
R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
R₆ is hydrogen, C₁₋₄alkyl, fluoro, 2-methoxy-ethoxy, chloro, cyano, or trifluoromethyl; and
R₇ is hydrogen or fluoro;
or an enantiomer, diastereomer, solvate, or pharmaceutically acceptable salt form thereof; and
wherein the second component is a mixture comprising fatty acid and polyethylene glycol monoesters and diesters, and optionally, fatty acid and glycerol monoesters, diesters and triesters;
wherein the fatty acid component of the fatty acid and polyethylene glycol monoesters and diesters, and of the fatty acid and glycerol monoesters, diesters and triesters, when present, comprises one or more saturated fatty acids having at least eight carbons.

43. The process of claim 42, wherein the solid dosage form comprises a capsule encapsulating the pharmaceutical formulation.

44. The process of claim 40, wherein the melt is formed by heating to a temperature above the drop point of the second component; and the cooling step is performed by cooling to below the drop point of the second component.

45. The process of claim 40, wherein the melt is formed by heating to a temperature of at least about 5° C. above the drop point of the second component, or at least about 10° C. above the drop point of the second component.

46. (canceled)
